(19) United States
(12) Patent Application Publication
(10) Pub. No.: US 2025/0177968 A1
(43) Pub. Date: Jun. 5, 2025

(54) TWO-DIMENSIONAL MATRIX DROPLET ARRAY

(71) Applicant: Abbott Laboratories, Abbott Park, IL (US)

(72) Inventors: Tomohiro Ikeda, Ichikawa-Shi (JP); Yousuke Omokawa, Tokyo (JP); Keisuke Yamaguchi, Tokyo (JP); Robert P. Luoma, Colleyville, TX (US); Pathik H. Soni, Chicago, IL (US); Jason N. Harris, Dallas, TX (US); Lei Qiao, Lake Bluff, IL (US); Lyle Yarnell, Richardson, TX (US); Dustin L. House, Addison, TX (US); Nazmus Sakib, Abbott Park, IL (US); Irsha Pardeshi, Abbott Park, IL (US)

(21) Appl. No.: 18/763,854

(22) Filed: Jul. 3, 2024

Related U.S. Application Data
(60) Provisional application No. 63/525,617, filed on Jul. 7, 2023.
(60) Provisional application No. 63/601,393, filed on Nov. 21, 2023.

(51) Int. Cl. B01L 3/00; G01N 33/543; G01N 33/68

(52) U.S. Cl. CPC B01L 3/502; G01N 33/54326; G01N 33/6878; B01L 2200/0647; B01L 2200/0689; B01L 2300/0829; B01L 2300/0858; B01L 2300/161; B01L 2400/043

(57) ABSTRACT

The present disclosure provides a device that may be used for a range of different assays such as immunoassays, nucleic acid analysis, metabolite analysis, clinical chemistry, and complete blood cell count. The device optionally contains a sample analysis region to analyze the samples processed in the device. The device comprises a top substrate bound to a bottom substrate wherein the top substrate bound to the bottom substrate forms two or more primary zones separated by one or more secondary zones, and wherein the top substrate has an opening in one or more of the primary zones.

### CROSS-REFERENCE TO RELATED APPLICATIONS

This application claims priority benefit to the filing dates of U.S. Provisional Patent Application Ser. No. 63/525,617, filed on Jul. 7, 2023, and U.S. Provisional Patent Application Ser. No. 63/601,393, filed on Nov. 21, 2023, the disclosures of which applications are herein incorporated by reference in their entirety.

### INTRODUCTION

Analyte analysis is usually performed by carrying out sample preparation step that is either performed manually or using complicated robotics. After sample preparation, the assaying of an analyte in the prepared sample further involves use of expensive and complicated systems for transporting the prepared sample to a machine that then performing analysis of an analyte in the prepared sample.

Devices that can be used to prepare a sample for multiple types of assays and assay the prepared sample are highly desirable in the field of analyte analysis. Such devices would offer a low cost option and would considerably increase the ease of performing analyte analysis, especially in clinical applications, such as point-of-care applications.

As such, there is an interest in devices for sample preparation because they allow for reduced sample volumes and reagent volumes, potential for higher sensitivity, and faster time to result.

### SUMMARY

The present invention is defined by the appendant claims.

The present disclosure provides a device that may be used for a range of different assays such as immunoassays, nucleic acid analysis, metabolite analysis, clinical chemistry, and complete blood cell count. The device optionally contains a sample analysis region to analyze the samples processed in the device.

In an aspect, the invention provides a device that comprises a top substrate bound to a bottom substrate wherein the top substrate bound to the bottom substrate forms two or more primary zones separated by one or more secondary zones, and wherein the top substrate has an opening in one or more of the primary zones.

In another aspect, the invention provides a sample processing device comprising a top substrate, a bottom substrate attached to the top substrate so as to form an interior volume, wherein the top substrate and/or bottom substrate substantially define a first plane, a sample processing region within the interior volume, wherein the sample processing region comprises two or more primary zones and one or more secondary zones, wherein each primary zone is separated from an adjacent primary zone in a direction substantially along the first plane by a secondary zone of the one or more secondary zones, wherein the top substrate comprises an opening into a respective primary zone of the two or more primary zones.

In other aspects, the invention provides methods of using the discussed devices and sample processing devices.

Also provided is an optional reagent delivery device that may be used with a version of the device. A pressure sample mixing device is also provided.

As will be discussed further below, the sample processing device of the invention may comprise an internal volume formed by the top substrate and bottom substrate when attached to one another. This internal volume may itself be split into, or comprise, different regions which are connected to one another. For instance, the device may comprise a sample processing region within, or forming part of, the internal volume. This sample processing region may be used to perform one or more processing steps on a sample attached to a microparticle as the microparticle is moved through the sample processing region.

The sample processing region may be connected to a sample analysis region, which sample analysis region can be used to analyze the processed sample. This analysis may be performed using external apparatus to, e.g. digitally image, the sample analysis region. In this context, a connection between regions is intended to mean that a microparticle can be moved between said regions, e.g. a path or channel exists for the microparticle to move from one region to another. The sample processing region may additionally or alternatively be connected to a sample mixing region. For instance, in use, a microparticle may be able to travel from a sample mixing region, into a sample processing region, and then into a sample analysis region.

### BRIEF DESCRIPTION OF THE DRAWINGS

FIG. 1 illustrates a top view of the top of the sample processing device according to one embodiment.

FIG. 2 illustrates a top view of the top of the sample processing device according to another embodiment.

FIG. 3 illustrates a top view of the top of the sample processing device according to another embodiment.

FIG. 4 illustrates a top view of the top of the sample processing device according to another embodiment.

FIG. 5 illustrates a top view of the top of the sample processing device according to another embodiment.

FIG. 6 illustrates a top view of the top of the sample processing device according to another embodiment.

FIG. 7 illustrates an isometric view of the top of the sample processing device according to one embodiment.

FIG. 8 illustrates an isometric view of the top of the sample processing device according to another embodiment.

FIG. 9 illustrates an enlargement of the sample analysis region according to one embodiment.

FIG. 10 illustrates an exemplary method of mixing the sample using the device according to one embodiment.

FIG. 11 illustrates an isometric view of the top of the sample processing device according to another embodiment.

FIG. 12 illustrates a top view of the top of the sample processing device according to another embodiment.

FIG. 13 illustrates a top view of the top of the sample processing device according to another embodiment.

FIG. 14 illustrates a bottom view of the top of the sample processing device, i.e., the top substrate, according to another embodiment.

FIG. 15 illustrates a bottom view of the top of the sample processing device, i.e., the top substrate, according to another embodiment.

FIG. 16 illustrate a cross-section two exemplary primary zones of the device according to an embodiment.

FIG. 17 illustrates an exemplary sample processing path according to an embodiment.

FIG. 18 illustrates an exemplary sample processing path according to an embodiment.

FIGS. 19A-19E illustrate an exemplary sample processing path according to an embodiment.

FIG. 20 illustrates an isometric view of the reagent delivery device according to an embodiment.

FIG. 21 illustrates a deconstructed view of the reagent delivery device according to an embodiment.

FIG. 22 illustrates a deconstructed view of a component of the reagent delivery device.

FIG. 23 illustrates a cross-section of the internal components of the reagent delivery device in the non-activated position according to an embodiment.

FIG. 24 illustrates a cross section of the internal components of the reagent delivery device in the activated position according to an embodiment.

FIG. 25 illustrates a cross-section of the internal components of a component of the reagent delivery device according to an embodiment.

FIGS. 26A-26D illustrate a cross-section of the primary zone with sample present.

FIGS. 27A-27B illustrate exemplary pad designs.

FIGS. 28A-28K illustrate exemplary fluid retention features.

FIG. 29 illustrates an exemplary sample mixing device.

FIGS. 30A-30C illustrates exemplary sample mixing devices.

FIG. 31 illustrates exemplary secondary features for fixed primary zones.

FIGS. 32A-32B. A. illustrates an exemplary method of mixing the sample using the device according to one embodiment. B. Illustrates a bottom view of the top of the sample processing device, i.e., the top substrate, according to another embodiment.

FIGS. 33A-33O. A-G. illustrates exemplary sample analysis regions. H-N. illustrates exemplary substrate retention features. O. illustrates an exemplary barrier feature.

FIG. 34 illustrates an exemplary sample analysis region of the embodiment depicted in FIG. 8 and FIG. 9.

FIGS. 35A-35H illustrate moving microparticles or microparticles and assisting particles across an array of wells and loading microparticles into wells of the array using a magnet in accordance with an aspect of the disclosed subject matter.

FIG. 36 is a diagram illustrating the magnet force upon a plurality of microparticles or microparticles and assisting particles.

FIGS. 37A-37C are diagrams of exemplary embodiments of the device.

### DETAILED DESCRIPTION OF THE INVENTION

A device is disclosed. The device optionally contains a sample analysis region to analyze the samples processed in the device. Also provided herein are exemplary methods for using the device. Also provided is an optional reagent delivery device that may be used in conjunction with the device. Also provided are sample mixing devices and methods.

Before the present invention is described in greater detail, it is to be understood that this invention is not limited to a particular embodiment described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an” and “the” include plural referents unless the context clearly dictates otherwise. Thus, for example, refer to “a primary zone” includes plurality of such primary zones and reference to “the well” includes reference to one or more wells and equivalents thereof known to those skilled in the art, and so forth.

All publications mentioned herein are incorporated herein by reference to disclose and describe the methods and/or materials in connection with which the publications are cited. The present disclosure is controlling to the extent there is a contradiction between the present disclosure and a publication incorporated by reference.

### DETAILED DESCRIPTION

Embodiments of the present disclosure relate to methods, and devices for analysis of analyte(s) in a sample. The sample may be a range of different samples including, without limitation, a biological sample, an environmental sample, a food sample, a water sample, etc. In some embodiments, the biological sample is a liquid sample or a liquid extract of a solid sample. Non-limiting examples of biological samples include bodily fluid, blood, veinous blood, capillary blood, serum, plasma, urine, saliva, sweat, sputum, semen, mucus, lacrimal fluid, tears, dermal fluid, lymph fluid, amniotic fluid, interstitial fluid, intestinal fluid, gastrointestinal fluid, lung lavage, spinal fluid, cerebrospinal fluid, feces, nasal mucus, virginal discharge, tissue, organ, or like. In some embodiments, tissues may include, but are not limited to, skeletal muscle tissue, liver tissue, lung tissue, kidney tissue, myocardial tissue, brain tissue, bone marrow, cervix tissue, skin, etc. In certain cases, the source of the sample may be an organ or tissue, such as a biopsy sample, which may be solubilized by tissue disintegration/cell lysis.

### Definitions

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of from about “2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e., an upper component is located at a higher elevation than a lower component in a given orientation, but these terms can change if the component is flipped. The terms “inlet” and “outlet” are relative to a fluid flowing through them with respect to a given structure, e.g., a fluid flows through the inlet into the structure and flows through the outlet out of the structure.

The terms “horizontal” and “vertical” are used to indicate direction relative to an absolute reference, i.e., ground level. However, these terms should not be construed to require structures to be absolutely parallel or absolutely perpendicular to each other. For example, a first vertical structure and a second vertical structure are not necessarily parallel to each other. The terms “top” and “bottom” are used to refer to surfaces where the top is always higher than the bottom relative to an absolute reference, i.e., the surface of the earth. The terms “upwards” and “downwards” are also relative to an absolute reference; upwards is always against the gravity of the earth while downwards is always towards the gravity of the earth.

“Comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Microbead” and “microparticle” are used herein interchangeably and refer to a substantially spherical solid support. The microbead or microparticle is a substantially spherical solid support that is influenced by a magnetic field such that the magnetic field can attract or repulse the microparticle or magnetic particle. A microbead or microparticle may occupy or settle in an array of wells, such as, for example, in an array of wells in a detection module. The microparticle and microbead may contain at least one specific binding member that binds to an analyte of interest and at least one detectable label. Alternatively, the microparticle and microbead may contain a first specific binding member that binds to the analyte and a second specific binding member that also binds to the analyte and contains at least one detectable label.

“Non-functional bead,” “helper bead,” and “assisting particle” are used interchangeably and refers to a substantially spherical assisting solid support, that is larger in diameter than a microparticle, which is configured to be chemically inert with respect to other components of an assay. As used herein, an assisting particle refers to a spherical particle which generally does not chemically interact with other particles (including a microparticle, conjugate, and/or reagent), but which is magnetic or paramagnetic. In certain exemplary embodiments, assisting particle may be coated so as to chemically interact with interferents, that is, any materials which would interfere with assay or analysis of an analyte of interest within the targeted sample. In such embodiments, the assisting particles can also improve binding efficiency of the microparticles including, for the purpose of illustration and not limitation, by binding with interferents. Additionally and alternatively, the shape of a solid support can be roughly spherical, though not limited to such shapes.

The term “adjacent” is used with respect to the zones and regions of the device and sample processing device. The term “adjacent” may mean that the adjacent entities neighbor one another, even if separated by another entity. For instance, if two primary zones are adjacent one another they may be neighboring one another, even if they are separated by, e.g. an intervening secondary zone. Similarly, two regions of the device may be described as adjacent if they neighbor one another even if they are separated by, e.g., a channel or opening. If the device is planar, e.g. has a width and length significantly greater than its thickness (such that the width and length define a first plane), adjacent may be taken to mean that the zones or regions in question are located next to each other in the width and/or length direction (e.g. along a plane of the device), e.g. they may be side by side in the width and/or length direction.

The term “height” is used in respect of the zones and regions of the device. The “height” may be measured as, e.g., the shortest distance between the interior surface of the top substrate and the interior surface of the bottom substrate at a given location. For instance, if the height of a given primary zone is to be measured, this may be measured as the shortest distance between the interior surface of the top substrate at that primary zone and the interior surface of the bottom substrate at that primary zone. If the device is planar, e.g. has a width and length significantly greater than its thickness (such that the width and length define a first plane), the height may be measured in a direction substantially parallel to the thickness (e.g. perpendicular to the first plane).

The assisting solid supports can be larger in diameter than the other support mediums within the storage region and configured so as to not chemically interact with any other components within the mixing region. Specifically, the diameter of the assisting solid supports (e.g. helper beads) can be at least 1%, at least 2%, at least 3%, at least 4%, at least 5%, at least 6%, at least 7%, at least 8%, at least 9%, at least 10%, at least about 11%, at least about 12%, at least about 13%, at least about 14%, at least about 15%, at least 20%, at least 25%, at least 30%, at least 35%, at least 50%, at least 75%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000% greater or larger than the diameter of other support mediums (e.g., microparticles).

The assisting particles can be configured such that they do not chemically bond or pair with other components of the targeted solution, such as the microparticles, target conjugates, and/or the target analyte. In certain exemplary embodiments, both microparticles and assisting particles can be magnetic, paramagnetic, or superparamagnetic particles (or any combination therein). In such exemplary embodiments, both microparticles and assisting particles, under the influence of a magnetic field or force, can form into chains of connected particles which facilitates mixing within the targeted solution.

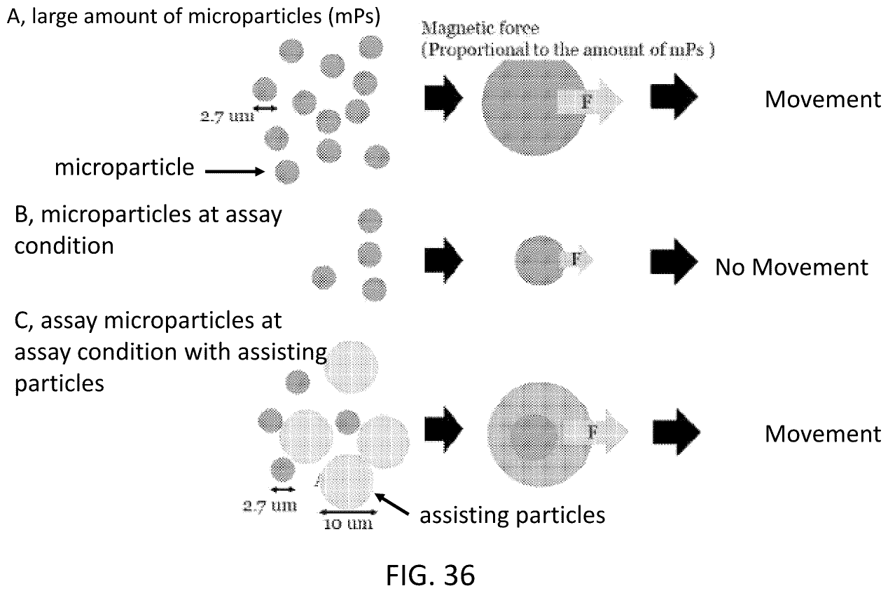

As shown in FIG. 36, such a configuration can be achieved by the inclusion of a plurality of assisting particles within the sample. These assisting particles are larger than the microparticles. In certain embodiments, the assisting particles can have a diameter of between about 5 μm and about 15 μm, and in certain exemplary embodiments, about 8 μm and about 12 μm, preferably about 10 μm. In certain exemplary embodiments, the assisting particles do not affect immunoreactions or other interactions of the microparticles with an analyte of interest, antigen, antibody, or other particle. The assisting particles can also be magnetic or paramagnetic, and thus contribute to the strength of the effective magnetic force which acts to move the sample (which contains both the assisting particles and microparticles). In certain exemplary embodiments, the assisting particles can include a negative surface charge, for example and not limitation, greater than or equal to −30 mV. The assisting particles can also be sized so as not to interfere with the assay of the targeted microparticles. This combination of active microparticles and inactive assisting particles can achieve the advantages of both 1) strong magnetic force coupling with the sample to enable movement through an assay surface and 2) high detection sensitivity resulting from a reduced (overall) amount of microparticles in the sample.

For purpose of example and not limitation, the microparticles and assisting particles can include a ratio of microparticles to assisting particles of between about 1:1 and 100:1. In some embodiments, the microparticles and assisting particles can include a ratio of microparticles to assisting particles of between about 1:1 and 50:1. In some embodiments, the microparticles and assisting particles can include a ratio of microparticles to assisting particles of between about 1:1 and 25:1. In some embodiments, the microparticles and assisting particles can include a ratio of microparticles to assisting particles of between about 1:1 and about 20:1. In some embodiments, the microparticles and assisting particles can include a ratio of microparticles to assisting particles of between about 1:1 and about 15:1. In some embodiments, the microparticles and assisting particles can include a ratio of microparticles to assisting particles of between about 1:1 and about 10:1. In some embodiments, the microparticles and assisting particles can include a ratio of microparticles to assisting particles of between about 1:1 and about 5:1. In some embodiments, the microparticles and assisting particles can include a ratio of microparticles to assisting particles of between about 5:1 and about 25:1 In some embodiments, the microparticles and assisting particles can include a ratio of microparticles to assisting particles of between about 10:1 and about 20:1. In some embodiments, the microparticles and assisting particles can include a ratio of microparticles to assisting particles of about 20:1. In some embodiments, the microparticles and assisting particles can include a ratio of microparticles to assisting particles of about 10:1. In some embodiments, the microparticles and assisting particles can include a ratio of microparticles to assisting particles of about 9:1. In some embodiments, the microparticles and assisting particles can include a ratio of microparticles to assisting particles of about 8:1. In some embodiments, the microparticles and assisting particles can include a ratio of microparticles to assisting particles of about 7:1. In some embodiments, the microparticles and assisting particles can include a ratio of microparticles to assisting particles of about 6:1. In some embodiments, the microparticles and assisting particles can include a ratio of microparticles to assisting particles of about 5:1. In some embodiments, the microparticles and assisting particles can include a ratio of microparticles to assisting particles of about 4:1. In some embodiments, the microparticles and assisting particles can include a ratio of microparticles to assisting particles of about 3:1. In some embodiments, the microparticles and assisting particles can include a ratio of microparticles to assisting particles of about 2:1. In some embodiments, the microparticles and assisting particles can include a ratio of microparticles to assisting particles of about 1:1.

“Component,” “components,” or “at least one component,” refer generally to a capture antibody, a detection reagent or conjugate, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum, whole blood, tissue aspirate, or plasma sample, in accordance with the methods described herein and other methods known in the art. Some components can be in solution or lyophilized for reconstitution for use in an assay.

“Label” or “detectable label” as used interchangeably herein refers to a moiety attached to a specific binding member or analyte to render the reaction between the specific binding member and the analyte detectable, and the specific binding member or analyte so labeled is referred to as “detectably labeled.” A label can produce a signal that is detectable by visual or instrumental means. Various labels include: (i) a tag attached to a specific binding member or analyte by a cleavable linker; or (ii) signal-producing substance, such as chromagens, fluorescent compounds, enzymes, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

“Specific binding partner” or “specific binding member” as used interchangeably herein refer to one of two different molecules that specifically recognizes the other molecule compared to substantially less recognition of other molecules. The one of two different molecules has an area on the surface or in a cavity, which specifically binds to and is thereby defined as complementary with a particular spatial and polar organization of the other molecule. The molecules may be members of a specific binding pair. For example, a specific binding member may include, but not limited to, a protein, such as a receptor, an enzyme, an antibody and an aptamer, a peptide, a nucleotide, oligonucleotide, a nucleic acid, a polynucleotide and combinations thereof.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

By “hybridizable” or “complementary” or “substantially complementary” it is meant that a nucleic acid (e.g. RNA, DNA) contains a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). Inosine (I) bases pair with cytosine/cytidine. In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA or RNA) base pairing with a sensor RNA) is considered complementary to both a U and to C. For example, when a G/U base-pair can be made at a given nucleotide position of a protein-binding segment (e.g., dsRNA duplex) of a sensor RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Analyte”, “target analyte”, “analyte of interest” as used interchangeably herein, refers to a substance, material or chemical constituent the presence, absence and/or amount of which is being analyzed in a biological sample obtained from a subject. In some aspects, the analyte is a biomolecule. Non-limiting examples of biomolecules include macromolecules such as, proteins, lipids, and carbohydrates. In certain instances, the analyte may be hormones, antibodies, growth factors, cytokines, enzymes, receptors (e.g., neural, hormonal, nutrient, and cell surface receptors) or their ligands, cancer markers (e.g., PSA, TNF-alpha), markers of myocardial infarction (e.g., troponin, creatine kinase, Creatinine kinase-cardiac muscle biomarker (CK-MB), B-type natriuretic peptide (also known as brain natriuretic peptide; BNP), N-terminal prohormone of brain natriuretic peptide (NT-proBNP) and the like), toxins, drugs (e.g., drugs of addiction), metabolic agents (e.g., including vitamins), and the like. Non-limiting examples of protein analytes include peptides, polypeptides, protein fragments, protein complexes, fusion proteins, recombinant proteins, phosphoproteins, glycoproteins, lipoproteins, or the like.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)2 fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety to disclose and describe the methods and/or materials in connection with which the publications are cited. The materials, methods, and examples disclosed herein are illustrative only and not intended to be limiting.

### 1. Overview

Provided herein is a device, also known as a sample processing device, that may be used for sample processing and analyte detection. To provide structure to the description of the device, first, the overall device design will be disclosed. Second, the uses of the device will be disclosed. Third, the benefits of the device will be disclosed. Following an overview of the basic design of the device, a detailed disclosure of the device's design will be disclosed which includes the arrangement of the sample processing region and the arrangement of the optional sample analysis region. Following the detailed disclosure of the device's design, an exemplary optional reagent delivery device will be disclosed. In some embodiments, bulk reagent delivery may be used in place of the optional reagent delivery device. Following the disclosure of the exemplary optional reagent delivery device, exemplary sample mixing devices and methods will be disclosed.

### I. Device Design

The device comprises a top substrate bound to a bottom substrate where the top substrate bound to the bottom substrate forms two or more primary zones separated by two or more secondary zones. Each primary zone contains an opening in the top substrate. In some embodiments, each secondary zone contains an opening in the top substrate. In some embodiments, each secondary zone does not contain an opening in the top substrate. The sample processing region is formed between the top and the bottom substrate. The sample processing region is unbounded throughout the region (i.e., the interior of the device containing the primary and secondary zones). In some embodiments, the top substrate and bottom substrate form a first distance between the top substrate and bottom substrate within the primary zones and a second distance between the top substrate and the bottom substrate within the secondary zones. In some embodiments, the first distance is less than the second distance.

The first distance may be less than the second distance because the pad of the primary zone protrudes into the sample processing region in at least some primary zones. This means that the distance from the pad to the interior surface of the bottom substrate is less than the distance from the interior surface of the top substrate to the interior surface of the bottom substrate in a secondary zone.

The top substrate may be substantially planar and the top substrate may have a width, length, and thickness, wherein the width and length are significantly greater than the thickness. The bottom substrate may be substantially planar and the bottom substrate may have a width, length, and thickness, wherein the width and length are significantly greater than the thickness. The width and length of the top substrate may be substantially identical to the width and length of the bottom substrate or the width and/or length may differ.

The device may be described as a “two-dimensional matrix droplet array” in that the device comprises a plurality of primary zones and secondary zones arranged substantially along two dimensions of a plane. At least some of the primary zones and secondary zones are configured to receive droplets of a fluid.

In some embodiments, the device comprises a first substrate and a second substrate positioned on the first substrate. The second substrate comprises a sidewall about at least a portion of a periphery of the second substrate where the first substrate, sidewall, and second substrate define a central chamber therebetween. The second substrate comprises a surface facing the central chamber comprising a plurality of recessed elements, and a plurality of protruding elements wherein primary zones are defined between a surface of the plurality of protruding elements facing the first substrate and a surface of the first substrate facing the second substrate and wherein secondary zones are defined between a surface of the recessed element facing the first substrate and the surface of the first substrate facing the second substrate. The second substrate has an opening in one or more of the primary zones.

In some embodiments, the device comprises a first substrate and a second substrate positioned on the first substrate. The first substrate comprises a sidewall about at least a portion of a periphery of the first substrate where the first substrate, sidewall, and second substrate define a central chamber therebetween. The second substrate comprises a surface facing the central chamber comprising a plurality of recessed elements, and a plurality of protruding elements wherein primary zones are defined between a surface of the plurality of protruding elements facing the first substrate and a surface of the first substrate facing the second substrate and wherein secondary zones are defined between a surface of the recessed element facing the first substrate and the surface of the first substrate facing the second substrate. The second substrate has an opening in one or more of the primary zones.

In some embodiments, the device comprises a first substrate, a spacer layer positioned on a surface of the first substrate wherein the spacer layer is disposed about at least a portion of a periphery of the first substrate, and a second substrate positioned on the first substrate. The first substrate comprises a sidewall about at least a portion of a periphery of the second substrate where the first substrate, sidewall, and second substrate define a central chamber therebetween. The second substrate comprises a surface facing the central chamber comprising a plurality of recessed elements, and a plurality of protruding elements wherein primary zones are defined between a surface of the plurality of protruding elements facing the first substrate and a surface of the first substrate facing the second substrate and wherein secondary zones are defined between a surface of the recessed element facing the first substrate and the surface of the first substrate facing the second substrate. The second substrate has an opening in one or more of the primary zones. In some embodiments, the spacer layer is selected from the group consisting of: an adhesive layer, a shim layer, and a raised feature layer. In some embodiments, the spacer layer is an adhesive layer. In some embodiments, the spacer layer is a shim layer. In some embodiments, the spacer layer is a first adhesive layer, a shim layer, and a second adhesive layer. In some embodiments, the spacer layer is a first adhesive layer, a raised feature layer, and a second adhesive layer.

The term “top substrate” may be used interchangeably with the term “second substrate”. The term “bottom substrate” may be used interchangeably with the term “first substrate”. The term “pad” may be used interchangeably with the term “protruding element”.

The device comprises a sample processing region in addition to an optional sample analysis region. The sample processing region is configured to allow the processing of a sample in a modular manner. By a “modular manner” it is meant that depending on the assay, the primary and/or secondary zones may be filled with reagents (e.g., wash buffer, lysis buffer, conjugation reagents, detection reagents, amplification reagent, etc.) particular for a given assay and the device is compatible with different types of assays (e.g., immunoassays, nucleic acid analysis, metabolite analysis, clinical chemistry, etc.) and different ways of performing the assays (e.g., modulating the number of wash steps, conjugation steps, etc.). The sample or components of the sample are moved from one primary zone to another primary zone using microparticles. In general, a sample is added to a primary zone in the sample processing region. Microparticles are added at the same time, before, or after the sample is added to the same primary and/or secondary zone that the sample is added to or will be added to. In some embodiments, the microparticles are already present in the primary and/or secondary zone to which the sample is added to. In some embodiments, the microparticles are microparticles and assisting particles. The sample or component of the sample is then bound to the microparticles and the microparticles or microparticles and assisting particles are moved using a magnetic field to a different primary zone. After sample processing is complete, the microparticles or microparticles and assisting particles are moved to a sample analysis region on the device or off the device.

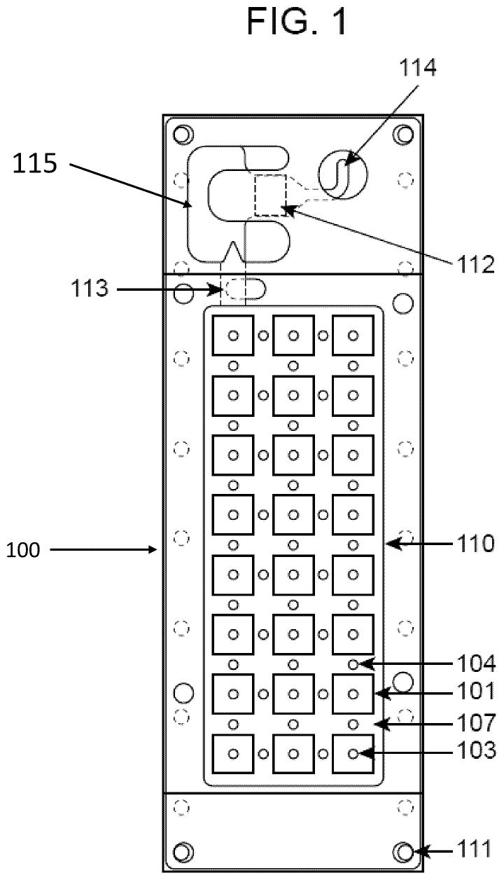

FIG. 1 discloses an illustration of an embodiment of the device. In this embodiment, the device 100 comprises a sample processing region 110. The sample processing region 110 is unbounded. By unbounded it is meant that there is no physical barrier separating the primary zones 101 and the secondary zones 107. While there is only a single element label for the primary zone 101, each square (101) is to represent an individual primary zone. While there is only a single element label for the secondary zone 107, each space between the squares is to represent an individual secondary zone. The sample processing region comprises a primary zone 101 that has an opening 103 in the primary zone. While there is only a single element label for the opening in the primary zone 103, each opening (103) in the square (101) is meant to represent an individual opening in the primary zone. The primary zone is adjacent to the secondary zone 107. The secondary zone may comprise an opening 104. While there is only a single element label for the opening in the secondary zone 107, each opening (104) in between the squares represents an individual opening in the secondary zone. The sample processing region 110 may be connected to a tertiary zone (i.e., the sample detection zone) 112 by a transition zone 113. The device may further comprise a quaternary zone 114 (i.e., a hydrophilic liquid well) that is connected to the tertiary zone. The top and the bottom substrate are bound by adhesive or clips 111. In some embodiments, the top substrate and bottom are bound by an adhesive layer between the top and bottom substrate. In some embodiments, the top and bottom substrate are bound using laser welding.

An alternative embodiment of FIG. 1 is disclosed below. In this embodiment, the device 100 comprises a sample processing region 110. The device 100 comprises a second substrate positioned on a first substrate. The second substrate comprises a surface facing a central chamber comprising a plurality of recessed elements, and a plurality of protruding elements. The primary zones 101 are defined between a surface of the plurality of protruding elements facing the first substrate and a surface of the first substrate facing the second substrate. The secondary zones 107 are defined between a surface of the plurality of recessed elements facing the first substrate and the surface of the first substrate facing the second substrate. The sample processing region 110 is unbounded. By unbounded it is meant that there is no physical barrier separating the primary zones 101 and the secondary zones 107. While there is only a single element label for the primary zone 101, each square (101) is to represent an individual primary zone. In some embodiments, the primary zones are discrete. By “discrete” it is meant that each primary zone is physically separated from each other primary zone. While there is only a single element label for the secondary zone 107, each space between the squares is to represent an individual secondary zone. In some embodiments, the secondary zones are connected. By “connected” it is meant that secondary zones are physically associated with each other. The sample processing region comprises a primary zone 101 that has an opening 103 in the primary zone. While there is only a single element label for the opening in the primary zone 103, each opening (103) in the square (101) is meant to represent an individual opening in the primary zone. The primary zone is adjacent to the secondary zone 107. The secondary zone may comprise an opening 104. While there is only a single element label for the opening in the secondary zone 107, each opening (104) in between the squares represents an individual opening in the secondary zone. The sample processing region 110 may be positioned adjacent to a sample analysis region comprising a tertiary zone (i.e., the sample detection zone) 112, a quaternary zone 114 (i.e., a hydrophilic liquid well), and a quinary zone 115 (i.e., a hydrophobic liquid well) wherein the tertiary zone 112, the quaternary zone 114, and the quinary zone 115 are connected. The sample analysis region is defined by the surface of the second substrate facing the first substrate and the surface of the first substrate facing the second substrate. The quinary zone 115 is located at a first end of the sample analysis region. The quinary zone comprises an opening spanning the second substrate. The quaternary zone 114 is located at a second end of the sample analysis region. The quaternary zone comprises a cylindrical opening spanning the second substrate. The tertiary zone 112 is located at a midpoint between the first end (e.g., the quinary zone 115) and the second end (e.g., the quaternary zone 115) of the of the sample analysis region. The sample analysis region may be connected to the sample processing region 110 by a transition zone 113. The second substrate positioned over the first substrate are bound together by adhesive or clips 111. In some embodiments, the second substrate and the first substrate are bound by an adhesive layer between the second substrate and the first substrate. In some embodiments, the second substrate and the first substrate are bound using laser welding.

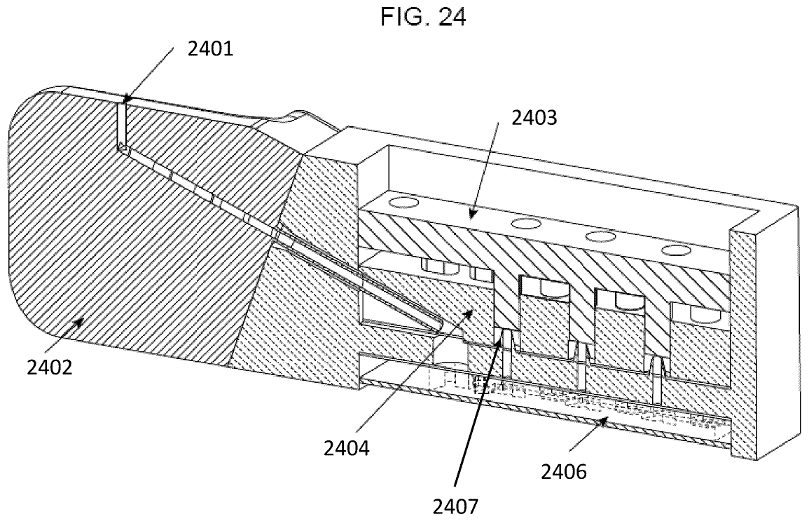

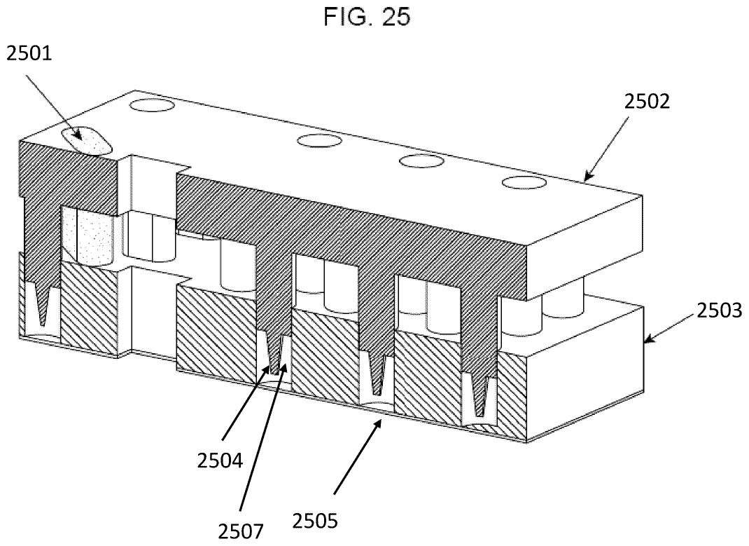

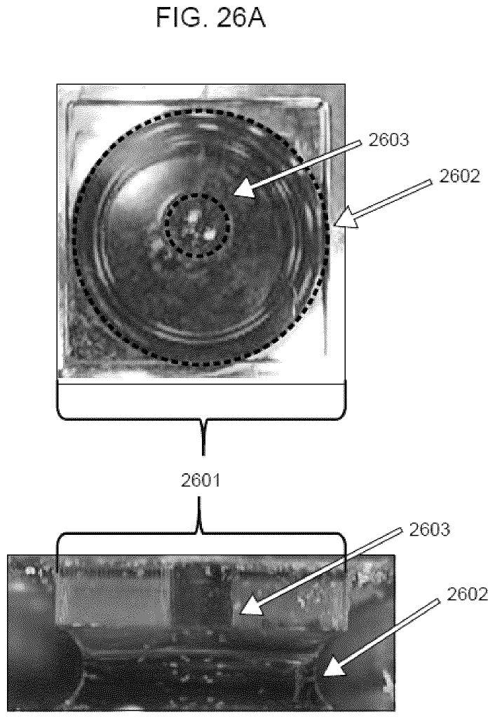

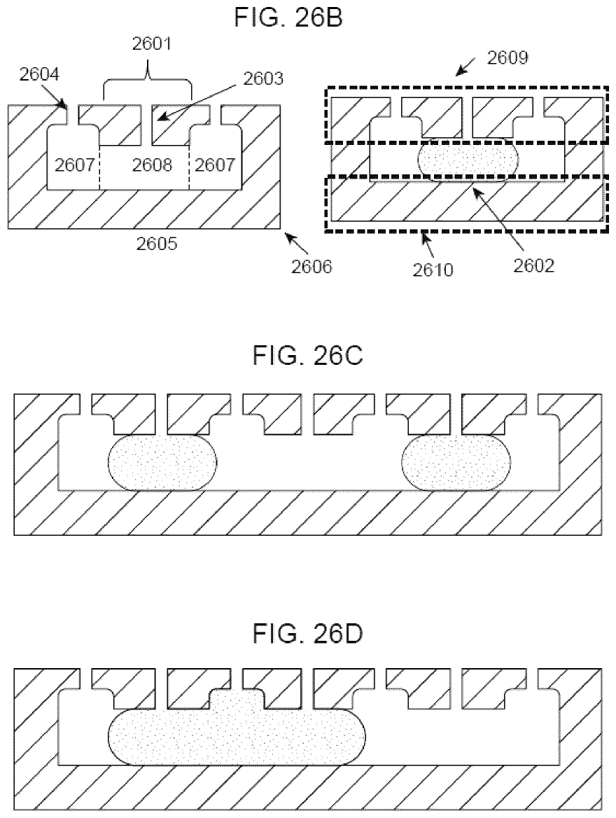

FIG. 26B discloses a schematic representation of a cross-section of the sample processing region in an embodiment. In FIG. 26B, the sample processing region 2605 contains a primary zone 2608 and two secondary zones 2607. The sample processing region 2605 is formed from the top substrate 2609 bound to the bottom substrate 2610. The primary zone 2608 contains an opening 2603. The secondary zone contains an opening 2604. The sample or reagent 2602 may be added through the opening 2603 of the primary zone 2608 wherein the sample or reagent 2602 is held in place through capillary forces generated by the opening 2603 and surface tension facilitated by the edges of a pad 2601 in the primary zone 2608. Generally, the secondary zones comprise air and serve as hydrophobic zones that assist in the holding of the sample or reagents in the primary zones. In some embodiments, the secondary zones also contain reagents or samples. In some embodiments, the secondary zones do not contain the sample or reagent. In some embodiments, a portion of the secondary zones contain the sample or reagent and a portion of the secondary zones do not contain the sample or reagents.

An alternative embodiment of FIG. 26B is described below. FIG. 26B discloses a schematic representation of a cross-section of the sample processing region in an embodiment. In FIG. 26B, the sample processing region 2605 contains a primary zone 2608 and two secondary zones 2607. The sample processing region 2605 is defined by a second substrate 2609 positioned on a first substrate 2610. Either the first substrate 2610 or the second substrate 2609 comprises a sidewall about at least a portion of a periphery of the first substrate 2610. The first substrate 2610, the sidewall, and the second substrate 2609 define a central chamber therebetween (i.e., the space occupied by 2607 and 2608). The second substrate comprises a surface (i.e., the interior portion of 2609) facing the central chamber (i.e., the space occupied by 2607 and 2608) comprising a protruding element 2601 and recessed elements (i.e., the upper portion of 2607). While only a single protruding element is shown it is to be understood that the device comprises a plurality of protruding elements. While only two recessed elements are shown it is to be understood that the device comprises a plurality of recessed elements. The primary zone 2608 is defined between the surface of the protruding element (i.e., the interior portion of 2601) facing the first substrate 2610 and a surface of the first substrate facing the second substrate (i.e., the interior portion of 2610). The secondary zones 2607 are defined between a surface of the recessed elements (i.e., the upper portion of 2607) and the surface of the first substrate facing the second substrate (i.e., the interior portion of 2610). The second substrate 2609 comprises an opening 2603 in the primary zone. The second substrate 2609 comprises an opening 2604 in the secondary zones. The sample or reagent 2602 may be added through the opening 2603 of the primary zone 2608 wherein the sample or reagent 2602 is held in place through capillary forces generated by the opening 2603 and surface tension facilitated by the protruding element 2601 in the primary zone 2608. Generally, the secondary zones comprise air and serve as hydrophobic zones that assist in the holding of the sample or reagents in the primary zones. In some embodiments, the secondary zones also contain reagents or samples. In some embodiments, the secondary zones do not contain the sample or reagent. In some embodiments, a portion of the secondary zones contain the sample or reagent and a portion of the secondary zones do not contain the sample or reagents.

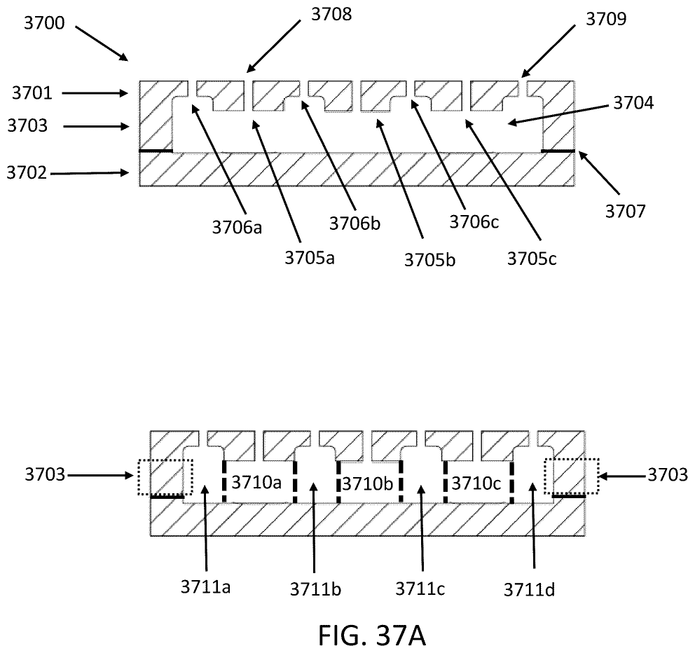

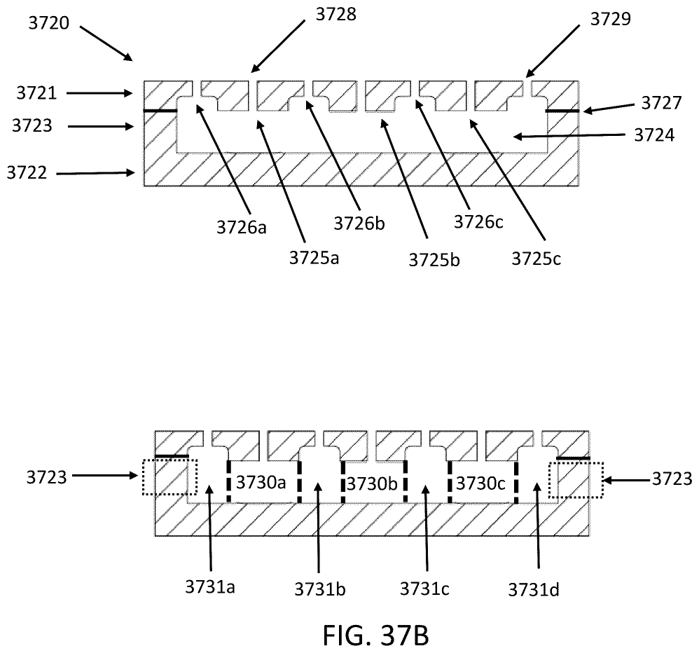

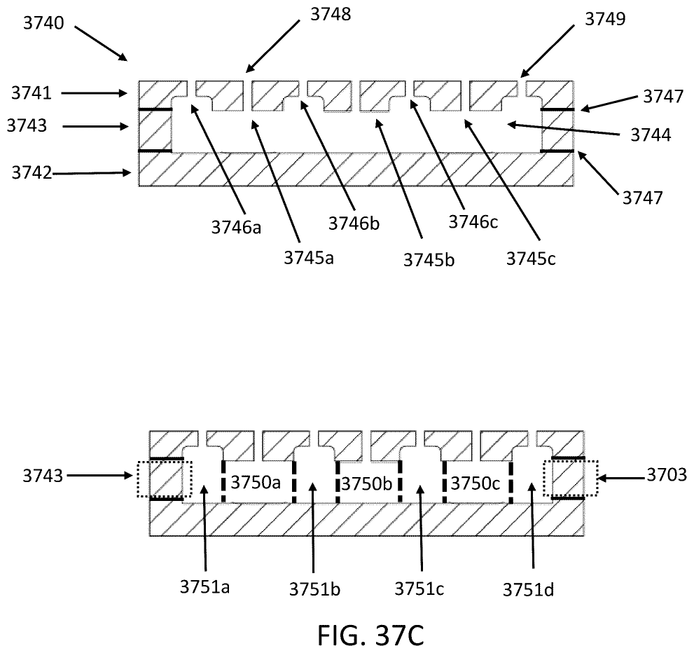

FIG. 37A discloses a schematic representation of a cross-section of the sample processing region of the device in an embodiment where the second substrate comprises a sidewall. In FIG. 37A, the device 3700 comprises a first substrate 3702 and second substrate 3701 positioned on the first substrate 3702. The second substrate 3701 comprises a sidewall 3703 about at least a portion of a periphery of the first substrate. The first substrate 3702, the sidewall 3703, and the second substrate 3701 define a central chamber 3704 therebetween. The second substrate comprises a surface facing the central chamber 3704 comprising a plurality of protruding elements (3705a, 3705b, and 3705c) and a plurality of recessed elements (3706a, 3706b, and 3706c). The border of the sidewall 3703 of the second substrate and the first substrate 3702 is defined by 3707 (black line). The primary zones (3710a, 3710b, and 3710c; bounded by thick black lines) are defined between the surface of the plurality of protruding elements (3705a, 3705b, and 3705c) facing the first substrate and the surface of the first substrate facing the second substrate (interior surface of 3702). The secondary zones (3711a, 3711b, 3711c, and 3711d; bounded by thick black lines) are defined between the surface of the plurality of recessed elements (3706a, 3706b, and 3706c) facing the first substrate and the surface of the first substrate facing the second substrate (interior surface of 3702). The second substrate 3701 has an opening 3708 in the primary zones. The second substrate 3701 has an opening 3709 in one or more of the secondary zones.

FIG. 37B discloses a schematic representation of a cross-section of the sample processing region of the device in an embodiment where the first substrate comprises a sidewall. In FIG. 37B, the device 3720 comprises a first substrate 3722 and second substrate 3721 positioned on the first substrate 3722. The first substrate 3722 comprises a sidewall 3723 about at least a portion of a periphery of the first substrate. The first substrate 3722, the sidewall 3723, and the second substrate 3721 define a central chamber 3724 therebetween. The second substrate comprises a surface facing the central chamber comprising a plurality of protruding elements (3725a, 3725b, and 3725c) and a plurality of recessed elements (3726a, 3726b, and 3726c). The border of the sidewall 3723 of the first substrate and the second substrate 3722 is defined by 3727 (black line). The primary zones (3730a, 3730b, and 3730c; bounded by thick black lines) are defined between the surface of the plurality of protruding elements (3725a, 3725b, and 3725c) facing the first substrate and the surface of the first substrate facing the second substrate (interior surface of 3722). The secondary zones (3731a, 3731b, 3731c, and 3731d; bounded by thick black lines) are defined between the surface of the plurality of recessed elements (3726a, 3726b, and 3726c) facing the first substrate and the surface of the first substrate facing the second substrate (interior surface of 3722). The second substrate 3721 has an opening 3728 in the primary zones. The second substrate 3721 has an opening 3729 in one or more of the secondary zones.

FIG. 37C discloses a schematic representation of a cross-section of the sample processing region of the device in an embodiment where the device comprises a spacer layer. In FIG. 37C, the device 3740 comprises a first substrate 3742 and a spacer layer 3743 positioned on the first substrate 3742 where the spacer layer is disposed about at least a portion of a periphery of the first substrate 3742. In some embodiments, the at least a portion of the periphery comprises at least two, at least three, or at least four peripheral sides of the second substrate. In some embodiments, the spacer layer is selected from the group consisting of: an adhesive layer, a shim layer, and a raised feature layer. In some embodiments, the spacer layer is an adhesive layer. In some embodiments, the spacer layer is a shim layer. In some embodiments, the spacer layer is a first adhesive layer, a shim layer, and a second adhesive layer. A second substrate 3741 positioned on the spacer layer 3743. The first substrate 3742, the spacer layer 3743, and the second substrate 3741 define a central chamber 3744 therebetween. The second substrate comprises a surface facing the central chamber comprising a plurality of protruding elements (3745a, 3745b, and 3745c) and a plurality of recessed elements (3746a, 3746b, and 3746c). The border of the spacer layer 3743, the first substrate 3742, and the second substrate 3741 is defined by 3747 (black lines). The primary zones (3750a, 3750b, and 3750c; bounded by thick black lines) are defined between the surface of the plurality of protruding elements (3745a, 3745b, and 3745c) facing the first substrate and the surface of the first substrate facing the second substrate (interior surface of 3742). The secondary zones (3751a, 3751b, 3751c, and 3751d; bounded by thick black lines) are defined between the surface of the plurality of recessed elements (3746a, 3746b, and 3746c) facing the first substrate and the surface of the first substrate facing the second substrate (interior surface of 3742). The second substrate 3741 has an opening 3748 in the primary zones. The second substrate 3741 has an opening 3749 in one or more of the secondary zones.

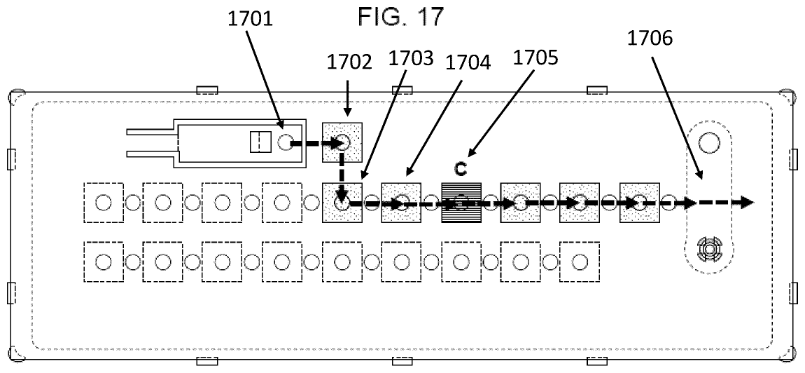

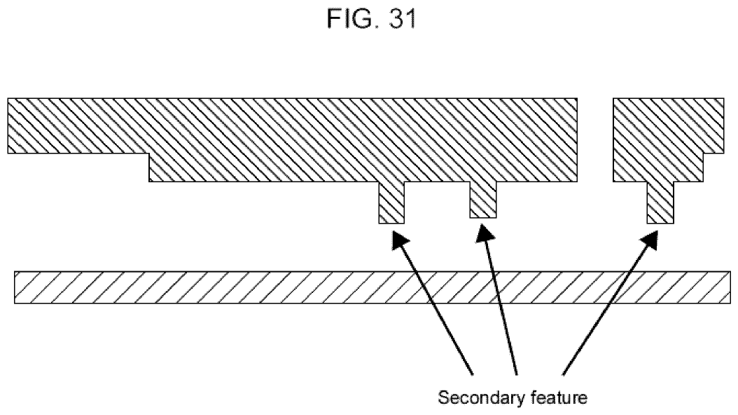

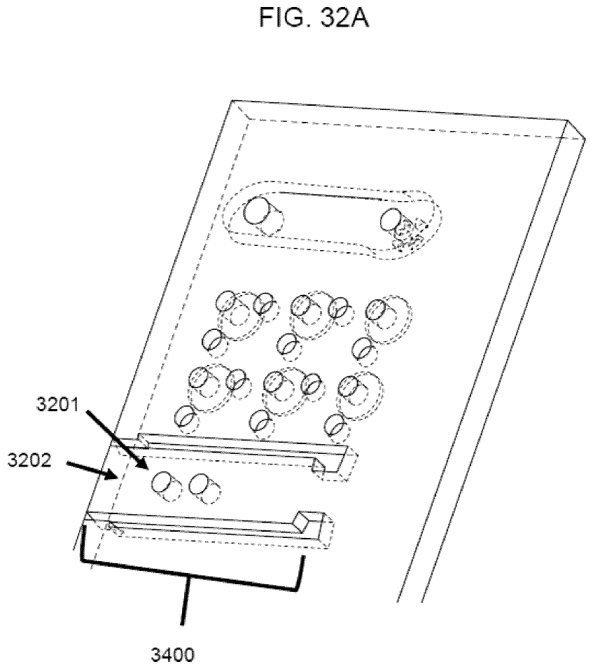

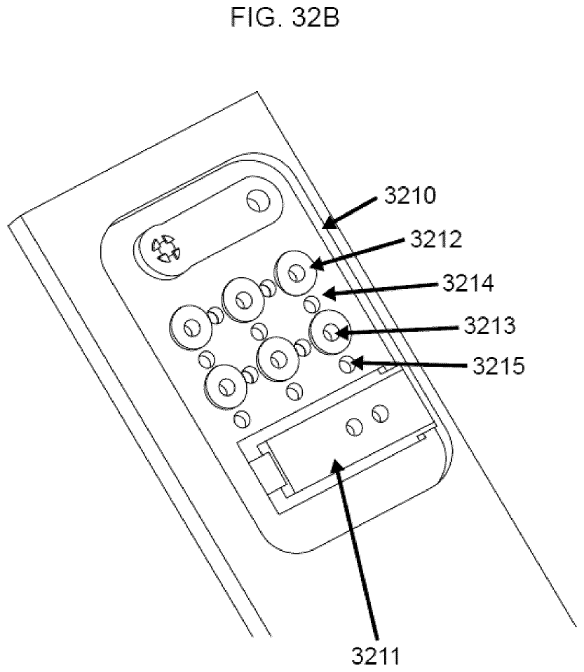

The schematics disclosed in FIG. 37A, FIG. 37B, and FIG. 37C may be applied to any of the devices disclosed herein. The schematics disclosed in FIG. 37A, FIG. 37B, and FIG. 37C may be applied to the devices disclosed in FIG. 1-15, FIG. 17-19, and FIG. 32.

As discussed above, the device may use capillary forces and surface tension to retain fluid within the various zones of the device. This effect occurs because of the because of the small size of the device and, e.g., the small dimensions associated with the primary and secondary zones. In particular, the behavior of a contained fluid is dominated by capillary forces and surface tension rather than gravity. This means, for instance, that an opening may provide capillary force to retain fluid within a given zone and that the edges of the pad may provide surface tension to retain fluid within a primary zone.

The top and bottom substrates of the present disclosure may be made of a range of different materials such that the materials facilitate the methods and designs disclosed herein. The material may be rigid or flexible. The rigidity and flexibility may be controlled both by the material used and the thickness of the material in the top and bottom substrate. In some embodiments, the top and bottom substrate are made of the same material. In some embodiments, the top and bottom substrates are made from different materials. In some embodiments, the entire top substrate is made of the same material. In some embodiments, the top substrate is made from a combination of materials where a portion of the top substrate is made from one material and a different portion of the top substrate is made from a different material. In some embodiments, the bottom substrate is made from a combination of materials where a portion of the bottom substrate is made from one material and a different portion of the bottom substrate is made from a different material. For instance, materials that find use in the present disclosure include, without limitation, glass, silicon, ceramic, metal, polymethyl methacrylate (PMMA), polystyrene (PS), polycarbonate (PC), cyclic olefin copolymer (COC), cyclic olefin polymer (COP), polypropylene (PP), polyurethane (PU), polytetrafluoroethylene (PTFE), polyvinylchloride (PVC), polydimethylsiloxane (PDMS), acrylonitrile butadiene styrene (ABS), poly(lactic acid) (PLA), thermoplastic PU, clear resin, polyethylene glycol diacrylate (PEGDA), etc.

In some embodiments, all or a portion of the top substrate is made of glass. In some embodiments, all or a portion of the top substrate is made of silicon. In some embodiments, all or a portion of the top substrate is made of ceramic. In some embodiments, all or a portion of the top substrate is made of metal. In some embodiments, all or a portion of the top substrate is made of polymethyl methacrylate (PMMA). In some embodiments, all or a portion of the top substrate is made of polystyrene (PS). In some embodiments, all or a portion of the top substrate is made of polycarbonate (PC). In some embodiments, all or a portion of the top substrate is made of cyclic olefin copolymer (COC). In some embodiments, all or a portion of the top substrate is made of polypropylene (PP). In some embodiments, all or a portion of the top substrate is made of polyurethane (PU). In some embodiments, all or a portion of the top substrate is made of polytetrafluoroethylene (PTFE). In some embodiments, all or a portion of the top substrate is made of polyvinylchloride (PVC). In some embodiments, all or a portion of the top substrate is made of polydimethylsiloxane (PDMS). In some embodiments, all or a portion of the top substrate is made of acrylonitrile butadiene styrene (ABS). In some embodiments, all or a portion of the top substrate is made of poly(lactic acid) (PLA). In some embodiments, all or a portion of the top substrate is made of thermoplastic PU. In some embodiments, all or a portion of the top substrate is made of clear resin. In some embodiments, all or a portion of the top substrate is made of polyethylene glycol diacrylate (PEGDA).

In some embodiments, all or a portion of the bottom substrate is made of glass. In some embodiments, all or a portion of the bottom substrate is made of silicon. In some embodiments, all or a portion of the bottom substrate is made of ceramic. In some embodiments, all or a portion of the bottom substrate is made of metal. In some embodiments, all or a portion of the bottom substrate is made of polymethyl methacrylate (PMMA). In some embodiments, all or a portion of the bottom substrate is made of polystyrene (PS). In some embodiments, all or a portion of the bottom substrate is made of polycarbonate (PC). In some embodiments, all or a portion of the bottom substrate is made of cyclic olefin copolymer (COC). In some embodiments, all or a portion of the bottom substrate is made of polypropylene (PP). In some embodiments, all or a portion of the bottom substrate is made of polyurethane (PU). In some embodiments, all or a portion of the bottom substrate is made of polytetrafluoroethylene (PTFE). In some embodiments, all or a portion of the bottom substrate is made of polyvinylchloride (PVC). In some embodiments, all or a portion of the bottom substrate is made of polydimethylsiloxane (PDMS). In some embodiments, all or a portion of the bottom substrate is made of acrylonitrile butadiene styrene (ABS). In some embodiments, all or a portion of the bottom substrate is made of poly(lactic acid) (PLA). In some embodiments, all or a portion of the bottom substrate is made of thermoplastic PU. In some embodiments, all or a portion of the bottom substrate is made of clear resin. In some embodiments, all or a portion of the bottom substrate is made of polyethylene glycol diacrylate (PEGDA).

### I(a). Sample Processing Region

The sample processing region of the devices disclosed herein have an arrangement of primary zones and secondary zones. In some embodiments, the primary zones are discrete. By “discrete” it is meant that each primary zone is physically separated from each other primary zone. In some embodiments, the secondary zones are connected. By “connected” it is meant that secondary zones are physically associated with each other. There is a range in the number of primary and secondary zones that is dependent on the particular embodiment being described. The arrangement of the primary and secondary zones is also variable and dependent on the particular embodiment discussed.

The device comprises a range in the number of primary and secondary zones. For example, the device may comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24 or more primary zones. The device may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, or 37 or more secondary zones. Each adjacent primary zone is separated by 1 secondary zone such that if there were 3 primary zones in a straight line there would be a 1^{st} secondary zone between the 1^{st} and 2^{nd} primary zone and 2^{nd} secondary zone between the 2^{nd} and 3^{rd} primary zone.

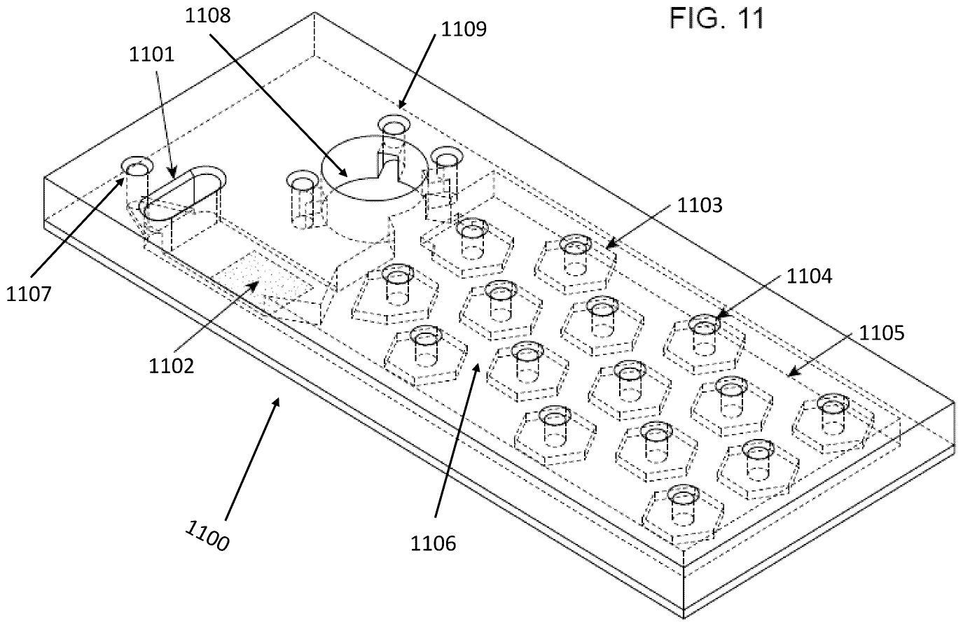

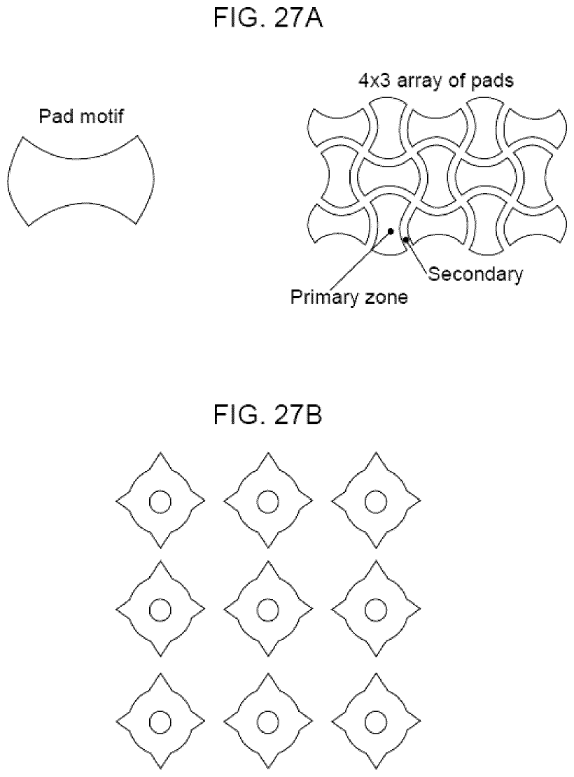

The primary and secondary zones may be arranged in a number of different patterns. For instance, the pattern may be a grid, lines, or a non-grid pattern. When the pattern is a grid, the grid may be 2×4, 2×5, 2×6, 2×7, 2×8, 2×9, 2×10, 3×4, 3×5, 3×6, 3×7, 3×8, 3×9, 3×10, 4×2, 4×3, 4×4, 4×5, 4×6, 4×8, 4×9, or 4×10 where the first number indicates the number of columns, and the second number indicates the number of rows. When the pattern is a line, the line may be 1×3, 1×4, 1×5, 1×6, 1×7, 1×8, 1×9, or 1×10. When the pattern is a non-grid, the non-grid may be in the form of a honeycomb pattern such as depicted in FIG. 11. When the pattern is a non-grid, the non-grid may be in the form of an irregular pattern such as depicted in FIG. 27A.

The pads or protruding elements in the primary zones may be separated by a particular distance such that fluids that may be contained within one primary zone does not bridge to another primary zone. The distance between primary zones may be a range of values. For instance, the distance between primary zones may be about 1 mm to about 2 mm. The range of distance or specific distance may be any intervening distance. For instance, the distance between primary zones may be at least about 1.00 mm, 1.01 mm, 1.02 mm, 1.03 mm, 1.04 mm, 1.05 mm, 1.06 mm, 1.07 mm, 1.08 mm, 1.09 mm, 1.10 mm, 1.11 mm, 1.12 mm, 1.13 mm, 1.14 mm, 1.15 mm, 1.16 mm, 1.17 mm, 1.18 mm, 1.19 mm, 1.20 mm, 1.21 mm, 1.22 mm, 1.23 mm, 1.24 mm, 1.25 mm, 1.26 mm, 1.27 mm, 1.28 mm, 1.29 mm, 1.30 mm, 1.31 mm, 1.32 mm, 1.33 mm, 1.34 mm, 1.35 mm, 1.36 mm, 1.37 mm, 1.38 mm, 1.39 mm, 1.40 mm, 1.41 mm, 1.42 mm, 1.43 mm, 1.44 mm, 1.45 mm, 1.46 mm, 1.47 mm, 1.48 mm, 1.49 mm, 1.50 mm, 1.51 mm, 1.52 mm, 1.53 mm, 1.54 mm, 1.55 mm, 1.56 mm, 1.57 mm, 1.58 mm, 1.59 mm, 1.60 mm, 1.61 mm, 1.62 mm, 1.63 mm, 1.64 mm, 1.65 mm, 1.66 mm, 1.67 mm, 1.68 mm, 1.69 mm, 1.70 mm, 1.71 mm, 1.72 mm, 1.73 mm, 1.74 mm, 1.75 mm, 1.76 mm, 1.77 mm, 1.78 mm, 1.79 mm, 1.80 mm, 1.81 mm, 1.82 mm, 1.83 mm, 1.84 mm, 1.85 mm, 1.86 mm, 1.87 mm, 1.88 mm, 1.89 mm, 1.90 mm, 1.91 mm, 1.92 mm, 1.93 mm, 1.94 mm, 1.95 mm, 1.96 mm, 1.97 mm, 1.98 mm, 1.99 mm, or at least about 2 mm.

The sample may be added to a fixed primary zone or a variable primary zone. By “fixed” primary zone it is meant that the primary zone for sample addition is fixed in place, i.e., a specific primary zone. In some embodiments, the fixed primary zone has one or more openings. For instance, there may be one or more, two or more, three or more, four or more, or five or more openings. By “variable” primary zone it is meant that the sample may be added to any of the primary zones present in the device. The terms “fixed primary zone” and “variable primary zone” are solely used for the purposes of referring to primary zones to which the sample is added and not primary zones to which the sample is not directly added.

In some embodiments, the sample processing region comprises a fixed primary zone. When the sample processing region comprises a fixed primary zone, the fixed primary zone may be larger than the primary zones. In some embodiments, the fixed primary zone is the same size as the primary zones. In some embodiments, the fixed primary zone is separate from the primary zones. In some embodiments, the fixed primary zone is a sample mixing region. In some embodiments, the fixed primary zone has one or more openings. For instance, there may be one or more, two or more, three or more, four or more, or five or more openings. In some embodiments, the sample processing region comprises two or more fixed primary zones. The fixed primary zones may have one or more secondary features that assist in the mixing of fluids contained therein. There may be a range in the number of secondary features in the fixed primary zone. For instance, there may be one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more. In some embodiments, the secondary features are according to FIG. 31. The secondary features may have any shape that assists in mixing that may occur in the fixed primary zone. Non-limiting examples of the shape include, without limitation, a rectangle, a circle, a triangle, a pentagon, a hexagon, a heptagon, an octagon, a decagon, a dodecagon, an amoeboid, a non-regular shape, etc.

In some embodiments, the fixed primary zone is formed from a laterally extended protruding element in the second substrate facing the first substrate wherein the laterally extended protruding element has a surface facing the first substrate that has a greater surface area than the surface of the plurality of protruding elements facing the first substrate. The laterally extended protruding element may be referred to as a tertiary protruding element. When the sample processing region comprises a fixed primary zone, the fixed primary zone may be larger than the primary zones. In some embodiments, the fixed primary zone has a surface facing the first substrate that has a greater surface area than the surface of the plurality of protruding elements facing the first substrate. In some embodiments, the fixed primary zone is separate from the primary zones. In some embodiments, the fixed primary zone is a sample mixing region. In some embodiments, the fixed primary zone is a cantilever wherein only a portion of the fixed primary zone is attached to the second substrate. In some embodiments, the fixed primary zone has one or more openings. For instance, there may be one or more, two or more, three or more, four or more, or five or more openings. In some embodiments, the sample processing region comprises two or more fixed primary zones. The fixed primary zones may have one or more secondary features that assist in the mixing of fluids contained therein. There may be a range in the number of secondary features in the fixed primary zone. For instance, there may be one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more. In some embodiments, the secondary features are according to FIG. 31. The secondary features may have any shape that assists in mixing that may occur in the fixed primary zone. Non-limiting examples of the shape include, without limitation, a rectangle, a circle, a triangle, a pentagon, a hexagon, a heptagon, an octagon, a decagon, a dodecagon, an amoeboid, a non-regular shape, etc.

In some embodiments, the sample processing region comprises a variable primary zone. The variable primary zone may be any of the primary zones present in the sample processing region. The sample may be added to the variable primary zone before, after, or at the same time as any other reagent added to the device.

### I(b). Sample Analysis Region

The optional sample analysis region of the devices disclosed herein comprises one or more sample detection zones. In some embodiments, the sample analysis region is present on the device. In some embodiments, the device does not contain a sample analysis region. When the device does not contain a sample analysis region, the analysis of the sample or analytes contained therein are analyzed off the device. There is both a range in the types of the sample detection zones and the number of sample detection zones present. The types of sample detection zones include, without limitation, wells or microwells, one or more chambers, nanopores, etc. In some embodiments, the sample analysis region comprises hydrophobic liquid and hydrophilic liquid wells.

In some embodiments, the sample analysis region has a first end laterally separated from a second end and is defined by the surface of the second substrate facing the first substrate and the surface of the first substrate facing the second substrate. In some embodiments, the surface of the second substrate facing the first substrate in the sample analysis region comprises an enlarged protruding element where a surface of the enlarged protruding element facing the first substrate has a greater surface area than a surface of the plurality of protruding elements facing the first substrate, and the enlarged protruding element extends from the first end to the second end. The enlarged protruding element may be referred to as a secondary protruding element.

In some embodiments, the sample analysis region comprises a sample detection zone comprising wells. The wells are generally designed to contain one or more microparticles. In some embodiments, the wells are only able to contain a single microparticle. In some embodiments, the wells are able to contain two microparticles. In some embodiments, the wells are able to contain three or more microparticles. There may be a range of wells present in the sample detection zone. For instance, the sample detection zone may contain 100 or more, 200 or more, 500 or more, 1000 or more, 2000 or more, 3000 or more, 4000 or more 5000 or more, 6000 or more, 7000 or more, 8000 or more, 9000 or more, 10000 or more, 20000 or more, 30000 or more, 40000 or more 50000 or more, 60000 or more, 70000 or more, 80000 or more, 90000 or more, 100000 or more, 200000 or more, 300000 or more, 400000 or more, 500000 or more, 600000 or more, 700000 or more, 800000 or more, 900000 or more, or 1000000 or more wells or microwells. In some embodiments, the sample detection zone comprises 100000 or more wells or microwells. In some embodiments, the sample detection zone comprises 300000 or more wells or microwells. In some embodiments, the portion of the sample detection region containing wells is optically transparent.

In some embodiments, the sample analysis region comprises a sample detection zone comprising a chamber. In some embodiments, the chamber is a reaction vessel. In some embodiments, the chamber is an imaging chamber. In some embodiments, the chamber is an imaging chamber and a reaction vessel. The chamber may be a range of different sizes and shapes such that the chamber is suitable for the detection of the sample or the analytes contained therein. The chamber may accommodate a reaction mixture having a volume of from 1 microliter to 1 milliliter. For example, the chamber may be sized to contain a volume of from about 1 to 10 μL, 10 to 50 μL, 50 to 100 μL, 100 to 200 μL, 200 to 300 μL, 300 to 400 μL, 400 to 500 μL, 500 to 600 μL, 600 to 700 μL, 700 to 800 μL, 800 to 900 μL, or from about 900 to 1000 μL. When the chamber is an imaging chamber, one or more of the sides of the chamber may be optically transparent. For instance, the top of the chamber, the bottom of the chamber, the sides of sides of the chamber, or any combination thereof may be optically transparent.

In some embodiments, the sample analysis region comprises a sample detection zone comprising nanopores. When the sample detection zone comprises nanopores, the sample comprise nucleic acids. In some embodiments, the nanopores are designed to only allow the translocation of a single nucleic acid at a time. The nucleic acid may be single- or double-stranded. When the nucleic acid is single-stranded, the nanopore has a diameter such that only a single single-stranded nucleic can be translocated. When the nucleic acid is double stranded, the nanopore has a diameter such that only a single double-stranded nucleic can be translocated. The nanopores may be any type of nanopore including, without limitation, biological nanopores, solid state nanopores, etc.

The sample detection zone of the present disclosure may be a single sample detection zone, multiple sample detection zones of the same type, or multiple sample detection zones of two or more different types.

When the sample detection zone is a single sample detection zone, the sample detection zone may be any of those described above or throughout the present disclosure. In some embodiments, the sample detection zone comprises wells. In some embodiments, the sample detection zone comprises microwells. In some embodiments, the sample detection zone comprises nanopores or nanochannels. In some embodiments, the sample detection zone comprises a chamber.

When the sample detection zone comprises wells or microwells, the wells or microwells may be specific to electrochemical detection, imaging analysis, or absorbance-based measurements. In some embodiments, the wells or microwells are specific to electrochemical detection. In some embodiments, the wells or microwells are specific to image analysis. In some embodiments, the wells or microwells are specific to absorbance-based measurements. In some embodiments, the wells or microwells are specific to electrochemical detection and image analysis. In some embodiments, the wells or microwells are specific to electrochemical detection and absorbance based measurements. In some embodiments, the wells or microwells are specific to image analysis and absorbance-based measurements. In some embodiments, the wells or microwells are specific to electrochemical detection, image analysis, and absorbance-based measurements.

When the sample detection zone comprises wells or microwells, the wells or microwells may be contained in a chamber or in an open region. In some embodiments, the wells or microwells are contained in a chamber. In some embodiments, the wells or microwells are in an open region.

When the sample detection zone comprises a chamber, imaging chamber, or reaction vessel, the chamber, imaging chamber, or reaction vessel may be specific to electrochemical detection, imaging analysis, or absorbance-based measurements. In some embodiments, the chamber or reaction vessel is specific to electrochemical detection. In some embodiments, the chamber, imaging chamber, or reaction vessel is specific to image analysis. In some embodiments, the chamber, imaging chamber, or reaction vessel is specific to absorbance-based measurements. In some embodiments, the chamber or reaction vessel is specific to electrochemical detection and image analysis. In some embodiments, the chamber, imaging chamber, or reaction vessel is specific to electrochemical detection and absorbance-based measurements. In some embodiments, the chamber, imaging chamber, or reaction vessel is specific to image analysis and absorbance-based measurements. In some embodiments, the chamber, imaging chamber, or reaction vessel is specific to electrochemical detection, image analysis, and absorbance-based measurements.

When the sample detection zone comprises nanopores or nanochannels, the nanopores or nanochannels may be contained in a chamber or in an open region. In some embodiments, the nanopores or nanochannels are contained in a chamber. In some embodiments, the nanopores or nanochannels are in an open region.

When the sample detection zone contains multiple sample detection zones it may be referred to as a combined sample detection zone. When the sample detection zone contains multiple sample detection zones, the sample detection zone may contain multiple sample detection zones of the same type or multiple sample detection zones of different types.

In some embodiments, the sample detection zone contains multiple sample detection zones of the same type, such as any of those described above. There is a range of different numbers of sample detections zones of the same type that may be in the combined sample detection zone. For example, the combined sample detection zone may contain 2 or more, 3 or more, 4 or more, 5 or more, 6 or more, 7 or more, 8 or more, 9 or more, or 10 or more sample detection zones. The sample detection zones in the combined sample detection zone may be in an open region or in discrete chambers that are linked in series, parallel, or in a grid-like fashion. In some embodiments, the sample detection zones in the combined sample detection zone are in an open region. In some embodiments, the sample detection zones in the combined sample detection zones are in discrete chambers that are linked in series. In some embodiments, the sample detection zones in the combined sample detection zones are in discrete chambers that are linked in parallel. In some embodiments, the sample detection zones in the combined sample detection zones are in discrete chambers that are linked in a grid-like fashion.

In some embodiments, the sample detection zone contains multiple sample detection zones of the same type, such as any of those described above. There is a range of different numbers of sample detections zones of the same type that may be in the combined sample detection zone. For example, the combined sample detection zone may contain 2 or more, 3 or more, 4 or more, 5 or more, 6 or more, 7 or more, 8 or more, 9 or more, or 10 or more sample detection zones. The sample detection zones in the combined sample detection zone may be in an open region or in discrete chambers that are linked in series, parallel or in a grid-like fashion. In some embodiments, the sample detection zones in the combined sample detection zone are in an open region. In some embodiments, the sample detection zones in the combined sample detection zones are in discrete chambers that are linked in series. In some embodiments, the sample detection zones in the combined sample detection zones are in discrete chambers that are linked in parallel. In some embodiments, the sample detection zones in the combined sample detection zones are in discrete chambers that are linked in a grid-like fashion.

In some embodiments, the sample detection zone contains multiple sample detection zones of different types, such as any of those described above. There is a range of different numbers of sample detections zones of the different types that may be in the combined sample detection zone. For example, the combined sample detection zone may contain 2 or more, 3 or more, 4 or more, 5 or more, 6 or more, 7 or more, 8 or more, 9 or more, or 10 or more sample detection zones. The combined sample detection zone may contain multiple sample detection zones of the same and different types. For instance, the combined sample detection zone may contain 2 sample detection zones comprising wells or microwells and two sample detection zones comprising chambers or reaction vessels. In some embodiments, the combined sample detection zone comprises microwells or wells, and nanopores or nanochannels. In some embodiments, the combined sample detection zone comprises microwells or wells, and chambers, imaging chambers, or reaction vessels. In some embodiments, the combined sample detection zone comprises nanopores or nanochannels, and chambers or reaction vessels. The sample detection zones in the combined sample detection zone may be in an open region or in discrete chambers that are linked in series, parallel, or in a grid-like fashion. In some embodiments, the sample detection zones in the combined sample detection zone are in an open region. In some embodiments, the sample detection zones in the combined sample detection zones are in discrete chambers that are linked in series. In some embodiments, the sample detection zones in the combined sample detection zones are in discrete chambers that are linked in parallel. In some embodiments, the sample detection zones in the combined sample detection zones are in discrete chambers that are linked in a grid-like fashion.

In some embodiments, the sample analysis region comprises hydrophobic liquid and hydrophilic liquid wells. When the sample analysis region comprises hydrophobic liquid and hydrophilic liquid wells, the sample detection zone comprises wells or microwells. The hydrophilic liquid well holds and/or delivers hydrophilic liquid to the wells or microwells. The hydrophobic liquid well holds and/or delivers hydrophobic liquid to the wells or microwells. The hydrophobic liquid and/or hydrophilic liquid may be pre-filled in, or added to, the device. When the hydrophobic liquid and/or hydrophilic liquid are added to the device, the addition may be performed manually or robotically. In general, hydrophilic liquid is added before the hydrophobic liquid is added. The substrate generally interacts with a component in the wells when microparticles and an analyte that is present such that the interaction produces a detectable signal. In some embodiments, the hydrophilic liquid is a substrate solution. The substrate solution may be any substrate solution that reacts with a specific binding member (e.g., the second specific binding member or the detectably labeled second specific binding member) to produce a detectable signal. In some embodiments, the hydrophobic liquid is oil. The oil may be any oil deemed useful. In certain cases, the hydrophobic liquid is selected based on its low affinity for water to decrease mixing of the hydrophobic liquid with the substrate solution. In certain cases, the hydrophobic liquid is an oil. In certain cases, the hydrophobic liquid is 3M FC-40 oil, a hydrocarbon oil, a vegetable oil, or silicone liquids (e.g., a silicone oil). In certain cases, the oil is a fluorocarbon oil. In certain cases, the oil is Novec 7500, FC-40, or Galden HT200.

### II. Uses of the Devices

The devices disclosed herein may be used for a number of different assay types and the assays may be performed in different ways. The different types of assays and the different ways that the assays are performed are based on the primary and secondary zones are configured (e.g., the reagents contained in each zone). The assays are conducted using a sample path where the microparticles are moved from one primary zone to another primary zone using a magnetic field. In some embodiments, the microparticles are microparticles and assisting particles. The different types of assays include, without limitation, immunoassays, nucleic acid analysis, metabolite analysis, clinical chemistry, complete blood count (CBC), etc.

In some embodiments, the device may be used to perform immunoassays. Any immunoassay may be utilized. The immunoassay may be an enzyme-linked immunoassay (ELISA), a competitive inhibition assay, such as forward or reverse competitive inhibition assays, or a competitive binding assay, for example. In some embodiments, a detectable label (e.g., such as one or more fluorescent labels one or more tags attached by a cleavable linker which can be cleaved chemically or by photocleavage) is attached to the capture antibody and/or the detection antibody.

In some embodiments, the device may be used to perform nucleic acid analysis. The device may employ various forms of nucleic acid analysis to detect analytes of interest, e.g., a nucleic acid, a non-nucleic acid containing a nucleic acid tag, or a nucleic acid produced from the analyte, including, without limitation, PCR, isothermal amplification, etc.

In some embodiments, the device may be used to perform metabolite analysis. In some cases, the clinical chemistry panels include metabolic panels. The metabolite analysis helps evaluate, for example, the body's electrolyte balance and/or the status of several major body organs. Examples of metabolite analyses that may be employed by the device include, but are not limited to, basic metabolic panel (BMP), comprehensive metabolic panel (CMP), electrolyte panel, lipid panel, liver panel, renal panel, and thyroid function panel. The basic metabolic panel (BMP) includes 8 tests, all of which are found in the CMP. The BMP provides information about the current health of kidneys and respiratory system as well as electrolyte and acid/base balance and level of blood glucose. The CMP measurement is used for liver and kidney health, level of blood glucose, acid/base balance in blood, fluid and electrolyte balance, and important blood proteins. In some cases, the CMP measures glucose, calcium, total amount of albumin and globulins, bilirubin, BUN (blood urea nitrogen), creatinine, albumin, sodium, potassium, bicarbonate, chloride, alkaline phosphatase (ALP), alanine transaminase (ALT), and aspartate aminotransferase (AST). The electrolyte panel is used to detect a problem with the body's fluid and electrolyte balance. For example, the electrolyte panel measures the blood levels of carbon dioxide, chloride, potassium, and sodium. The lipid panel is used to assess a subject's risk of developing cardiovascular disease. For example, the lipid panel measures the amount of cholesterol and other fats in blood, such as total cholesterol, LDL (low-density lipoprotein), HDL (high-density lipoprotein), and triglycerides. The liver panel (hepatic function panel) is used to screen for, detect, evaluate, and monitor acute and chronic liver inflammation (hepatitis), liver disease and/or damage. The liver panel measures different enzymes, proteins, and other substances made by liver. For example, the liver panel includes albumin, total protein, ALP, ALT, AST, gamma-glutamyl transferase (GGT), bilirubin, Lactate dehydrogenase (LD), Prothrombin time (PT). The renal panel (kidney function panel) includes tests such as albumin, creatinine, BUN, eGFR to evaluate kidney function. The thyroid Function Panel is used to evaluate thyroid gland function and to help diagnose thyroid disorders. The thyroid function panel measure thyroid hormone such as thyroxine (T4), triiodothyronine (T3), and thyroid stimulating hormone (TSH). In some cases, a high TSH level indicates that the thyroid gland is not making enough thyroid hormone (primary hypothyroidism). The opposite situation, in which the TSH level is low, usually indicates that the thyroid is producing too much thyroid hormone (hyperthyroidism). In other cases, the finding of an elevated TSH and low free T4 (FT4) or free T4 index (FTI) indicates primary hypothyroidism due to disease in the thyroid gland. A low TSH and low FT4 or FTI indicate hypothyroidism due to a problem involving the pituitary gland. A low TSH with an elevated FT4 or FTI is found in individuals who have hyperthyroidism. These clinical chemistry panels are well known in the art and are further described in the assay portion of the present disclosure.

In some embodiments, the device may be used to perform clinical chemistry. In certain cases, clinical chemistry may involve detection of electrochemical species or chromogenic reaction product generated by action of an enzyme on a substrate. For example, the substrate may be an analyte present in a sample and the enzyme may be specific for the analyte and may catalytically react with the analyte to generate an electrochemical species or a colored reaction product. In other cases, clinical chemistry may involve capturing the analyte using a first binding member to generate a first complex comprising the analyte and the first binding member; contacting the complex with a second binding member, that binds to the analyte, to generate a second complex comprising the analyte, the first binding member, and the second binding member. The second binding member is conjugated to an enzyme that generates an electrochemical species or chromogenic reaction product upon exposure to a suitable substrate.

In some embodiments, the device may be used to perform complete blood counts of blood cells or blood cell types. Blood cells and blood cell types that may be detecting by the devices disclosed herein include, without limitation, red blood cells, hemoglobin, white blood cells (including neutrophils, lymphocytes, monocytes, eosinophils, and basophils), platelets, reticulocytes, and nucleated red blood cells. Various measurements of different blood components may be performed, including, but not limited to, cell count, cell size, cell complexity, granularity, hematocrit, mean corpuscular volume, mean corpuscular hemoglobin, and mean corpuscular hemoglobin concentration. In some embodiments, the above disclosed measurements may be performed using stain independent methods in the absence of histological staining.

In order to perform different types of assays or perform the assays in different ways, the primary zones may be filled with reagents. The reagents may be added to the primary zones may be added manually by the user, robotically by a system employing the device, or through the use of a reagent delivery device. In some embodiments, reagents are added to the secondary zones in addition to the primary zones.

FIG. 26A-26D illustrates a cross-section of the sample processing region where the sample and/or reagents are added. FIG. 26A and FIG. 26B illustrates a simplified depiction of a sample processing region where the sample processing region contains one primary zone and two secondary zones and the primary zone is filled with a sample or reagent. The primary zone 2608 contains an opening 2603. The secondary zone 2607 contains an opening 2604. The sample or reagent 2602 may be added through the opening 2603 of the primary zone 2608 wherein the sample or reagent 2602 is held in place through capillary forces generated by the opening 2603 and surface tension facilitated by the edges of a square pad 2601 in the primary zone 2608.

FIG. 26C illustrates a simplified depiction of a sample processing region where the sample processing region contains three primary zones and four secondary zones and two of the primary zones are filled with sample or reagent.

FIG. 26D illustrates a simplified depiction of a sample processing region where the sample processing region contains three primary zones and four secondary zones and two of the primary zones and one of the secondary zones are filled with sample or reagent. In this embodiment, the sample or reagent is added through the opening in two of the primary zones and one of the secondary zones. The addition of the sample or reagent in three adjacent zones (i.e., two primary zones and one secondary) causes the zones to merge and create a singular zone of increased volume.

The primary zones of the present disclosure are capable of holding a range of different volumes while retaining the volume within the primary zone without spilling into the secondary zone or beyond. For instance, the primary zone may hold about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 μL of fluid. In some embodiments, the primary zone is capable of holding between 10-24 μL of fluid.

When the primary and the adjacent secondary zone are filled with the sample or fluid, the primary and the adjacent secondary zone are capable of holding a range of different volumes while retaining the volume within the primary and secondary zone without spilling into another zone. For instance, the primary and secondary zone collectively may hold about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 μL of fluid. In some embodiments, the primary and secondary zone is capable of holding between 20-50 μL of fluid.

When two primary and one secondary zones are filled with sample or fluid, the two primary and one secondary zones are capable of holding a range of different volumes while retaining the volume within the primary and secondary zone without spilling into another zone. For instance, the two primary and one secondary zones collectively may hold about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or about 150 μL of fluid. In some embodiments, the primary and secondary zone is capable of holding between 30-150 μL of fluid.

The assays used with the device are conducted using a sample path where the microparticles or microparticles and assisting particles are moved from one primary zone to another primary zone using a magnetic field. Exemplary sample paths are disclosed in FIG. 17-19 using different embodiments of the device.

In FIG. 17, an exemplary sample path is shown. The sample and the microparticles are present in a fixed primary zone 1701 (e.g., a sample mixing zone). In some embodiments, microparticles are microparticles and assisting particles. The microparticles or microparticles and assisting particles are moved (black arrow) from the fixed primary zone to a primary zone 1702 containing a reagent (e.g., wash buffer). The microparticles or microparticles and assisting particles are then moved through two additional primary zones containing reagent 1703 and 1704 to a fourth primary zone containing reagent (e.g., conjugate) 1705. The microparticles or microparticles and assisting particles are eventually moved from the fourth primary zone 1705 to the sample analysis region 1706.

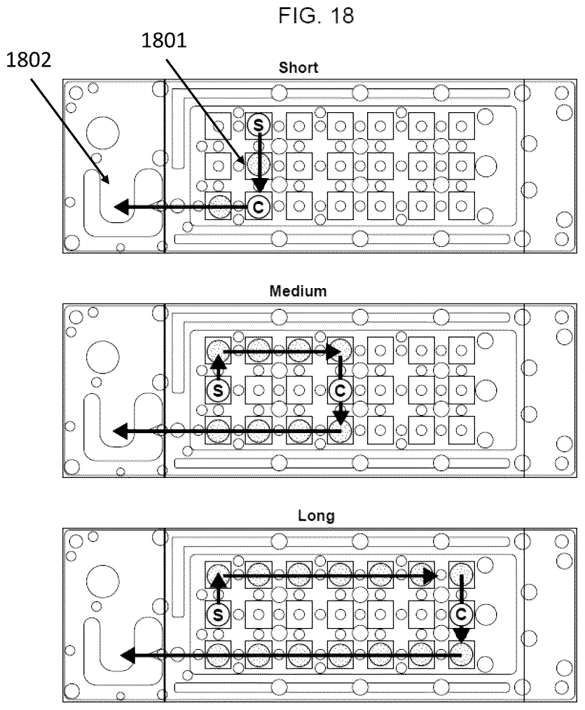

In FIG. 18, discloses short, medium, and long sample paths using the same exemplary device. In the short sample path, the sample and the microparticles or microparticles and assisting particles are present in a variable primary zone(S) and the microparticles or microparticles and assisting particles are then moved through a primary zone 1801 containing reagent (e.g., wash buffer) to a primary zone contain reagent (C; e.g., conjugate). The microparticles or microparticles and assisting particles are then moved from the primary zone containing reagent (C) through another primary zone and finally end in the sample analysis region 1802. In the medium path, the sample and the microparticles or microparticles and assisting particles are present in a variable primary zone(S) and the microparticles or microparticles and assisting particles are moved through four primary zones containing reagent (e.g., wash buffer) to a primary zone containing reagent (C; e.g., conjugate). The microparticles or microparticles and assisting particles are then moved through an additional four primary zones containing reagent (e.g., wash buffer) and finally end in the sample analysis region. In the long sample path, the sample and the microparticles are present in a variable primary zone(S) and the microparticles are moved through seven primary zones containing reagent (e.g., wash buffer) to a primary zone containing reagent (C; e.g., conjugate). The microparticles or microparticles and assisting particles are then moved through an additional seven primary zones containing reagent (e.g., wash buffer) and finally ending in the sample analysis region.

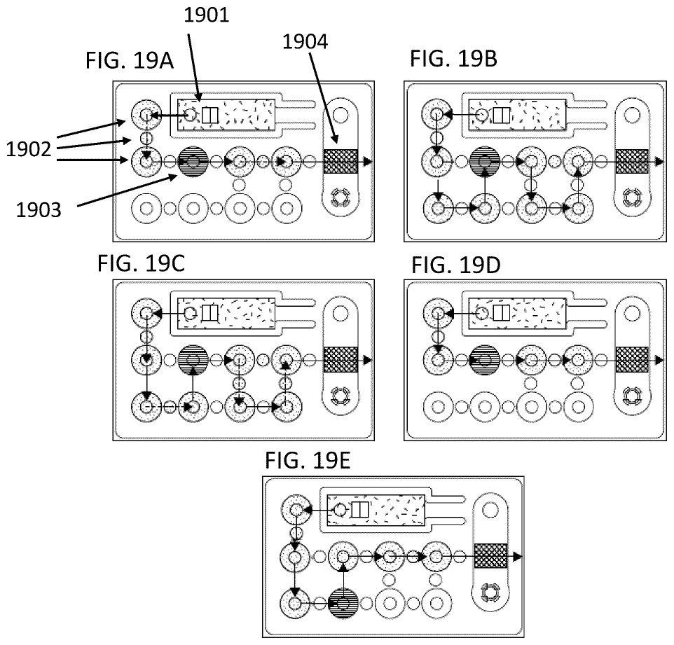

FIG. 19, discloses exemplary paths through an embodiment of the device. In FIG. 19A, the sample and the microparticles or microparticles and assisting particles are present in a fixed primary zone 1901 (e.g., a mixing zone) and moved through two primary and one secondary zones merged with reagent (e.g., merged zone; spotted) 1902 to a primary zone containing a reagent 1903 (dark stripped; e.g., conjugate). The microparticles or microparticles and assisting particles are then moved through another two primary and one secondary zones merged (dotted) with reagent to finally end in the sample analysis region 1904. In FIG. 19B, the sample and the microparticles or microparticles and assisting particles are present in a fixed primary zone and moved through four primary zones containing reagent (dotted, e.g., wash buffer) to a primary zone containing regent (dark stripped; e.g., conjugate). The microparticles or microparticles and assisting particles are then moved through four primary zones with reagent (spotted) to finally end in the sample analysis region. In FIG. 19C, the sample and the microparticles or microparticles and assisting particles are present in a fixed primary zone and moved through two merged zones containing reagent (dotted, e.g., wash buffer) to a primary zone containing regent (dark stripped; e.g., conjugate). The microparticles or microparticles and assisting particles are then moved through two merged zones with reagent (spotted) to finally end in the sample analysis region. In FIG. 19D, the sample and the microparticles or microparticles and assisting particles are present in a fixed primary zone and moved through two primary zones containing reagent (dotted, e.g., wash buffer) to a primary zone containing regent (dark stripped; e.g., conjugate). The microparticles or microparticles and assisting particles are then moved through two primary zones with reagent (spotted) to finally end in the sample analysis region. In FIG. 19E, the sample and the microparticles or microparticles and assisting particles are present in a fixed primary zone and moved through three primary zones containing reagent (dotted, e.g., wash buffer) to a primary zone containing regent (dark stripped; e.g., conjugate). The microparticles or microparticles and assisting particles are then moved through three primary zones with reagent (spotted) to finally end in the sample analysis region.

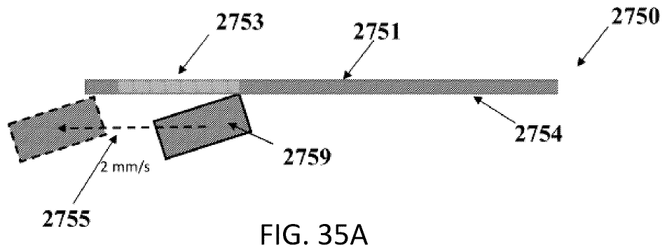

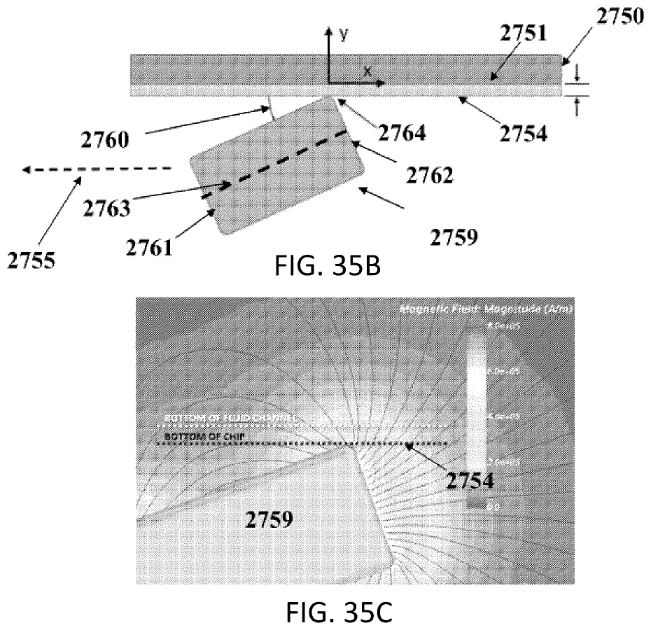

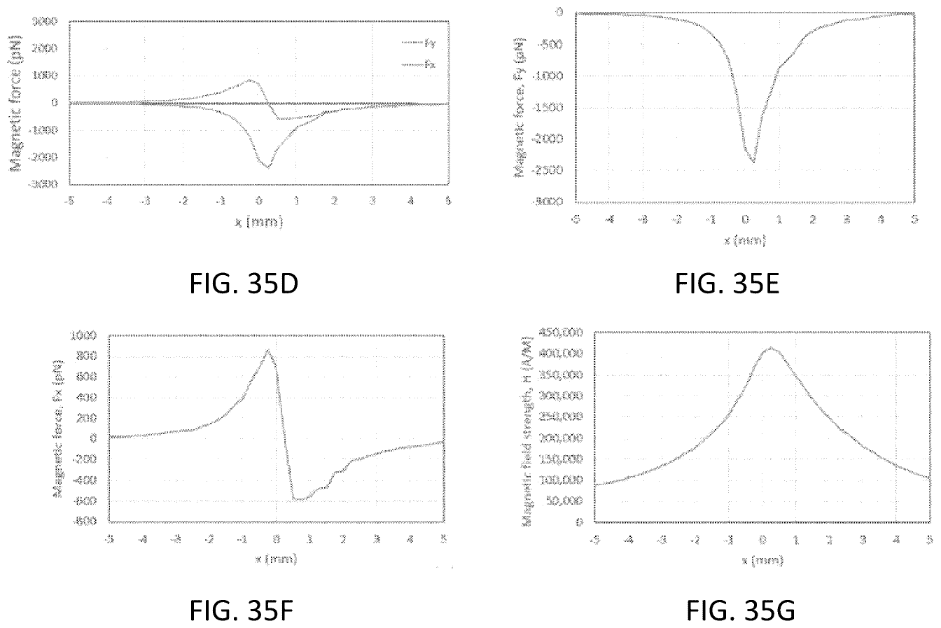

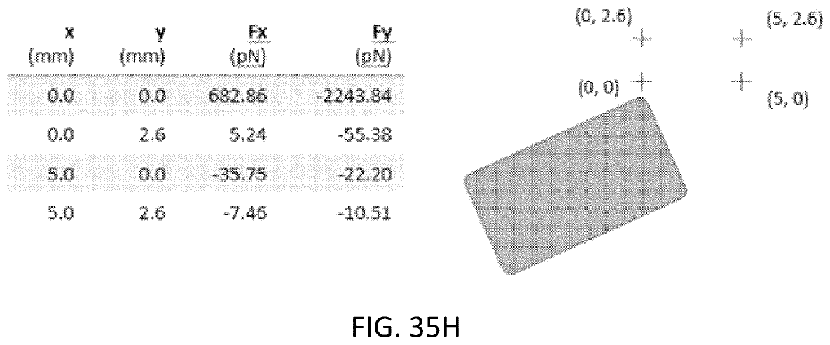

For purpose of illustration and example and not limitation, reference is made to the exemplary sample detection region 2750 depicted in FIG. 35A. In an embodiment, the sample detection region 2750 comprises wells 2753 defined therein. The wells 2753 have a well size. As described above and throughout, microparticles or microparticles and assisting particles can be moved across the array of wells 2753. In some embodiments, the microparticles include (i) a plurality of microparticles, each microparticle having a microparticle diameter smaller than the well size, and (ii) a plurality of assisting particles, each assisting particle having an assisting particle diameter larger than the well size. Specifically, the diameter of the assisting particle (e.g. helper beads) can be at least 1%, at least 2%, at least 3%, at least 4%, at least 5%, at least 6%, at least 7%, at least 8%, at least 9%, at least 10%, at least about 11%, at least about 12%, at least about 13%, at least about 14%, at least about 15%, at least 20%, at least 25%, at least 30%, or at least 35% greater or larger than the diameter of the well size. Moving the microparticles or microparticles and assisting particles across the array of wells 2753 further includes seeding a plurality of the microparticles or microparticles and assisting particles into wells of the array.

As embodied herein, moving the microparticles or microparticles and assisting particles across the array of wells 2753 includes moving a magnet 2759 along the array of wells. The magnet can be moved along the array of wells in any suitable configuration. For example and not limitation, the magnet can be located above the sample analysis region 2750. Additionally or alternatively, the magnet can be located adjacent to the sample analysis region. Additionally or alternatively, and as embodied herein, the magnet can be located below the sample analysis region 2750.

The position of the magnet relative to the sample analysis region 2750 and the array of wells 2753 can be selected based on the desired strength of the magnetic field to be applied to the microparticles or microparticles and assisting particles to move the microparticles or microparticles and assisting particles across the array of wells 2753. For purpose of example, and as described further herein, the distance between the magnet and the microparticles or microparticles and assisting particles can be selected based on the desired strength of the magnetic field to be applied to the microparticles or microparticles and assisting particles. For purpose of example and as embodied herein, the magnet can be moved along the array of wells at a magnet distance defined between a bottom surface 2754 of the array of wells and the magnet 2759. For example and not limitation, the magnet distance can be between about 0 mm and about 10 mm. Additionally or alternatively, and as embodied herein, the magnet can be moved along the array of wells 2753 with the magnet 2759 in contact with a bottom surface 2754 of the array of wells.

As further embodied herein, the shape and orientation of the magnet can also be selected to achieve the desired magnetic field. For example and not limitation, the magnet 2759 can be angled relative to the bottom surface 2754 of the array of wells as the magnet is moved along the bottom surface of the array of wells. For illustration and example and not limitation, reference is made to the exemplary sample analysis region 2750 depicted in FIG. 35B. A magnet axis 2763 can be defined between a first magnet end 2761 and a second magnet end 2762, and as the magnet 2759 is moved along the bottom surface 2754 of the array of wells. For purpose of example and not limitation, the magnet axis 2763 can be positioned at an angle 2760 of between about 0 degrees and about 80 degrees relative to the bottom surface of the array of wells. For example and not limitation, the magnet axis 2763 can be positioned at an angle 2760 of between about 10 degrees and about 30 degrees. For example and not limitation, and as embodied herein, the magnet axis 2763 can be positioned at an angle 2760 of about 20 degrees.

Additionally or alternatively, the magnetic element can be moved along the array of wells to achieve desired movement and seeding of microparticles or microparticles and assisting particles in the array of wells. For purpose of example and as embodied herein, the magnet 2759 can be moved in a direction 2755 parallel to the bottom surface 2754 of the array of wells. Additionally or alternatively, the magnetic element can be moved along the array of wells at any suitable speed to achieve desired movement and seeding of microparticles in the array of wells. For example, the speed of the magnet can be selected to achieve desired process times and microparticle loss during movement of the microparticles or microparticles and assisting particles across the array. For purpose of example and not limitation, magnet 2759 can be moved in a direction 2755 parallel to a bottom surface 2754 of the array of wells 2753 at a speed of between about 0.3 mm/s and about 10 mm/s. Additionally or alternatively, magnet 2759 can be moved in a direction 2755 parallel to a bottom surface 2754 of the array of wells 2753 at a speed of between about 0.3 mm/s and about 6 mm/s. Additionally or alternatively, magnet 2759 can be moved in a direction 2755 parallel to a bottom surface 2754 of the array of wells 2753 at a speed of between about 2 mm/s and about 6 mm/s. Additionally or alternatively, magnet 2759 can be moved in a direction 2755 parallel to a bottom surface 2754 of the array of wells 2753 at a speed of between about 4 mm/s and about 6 mm/s. For purpose of example and as embodied herein, the magnet 2759 can move in direction 2755 at an angle 2760 as described above, and the direction 2760 can be selected to align with the direction of the acute angle defined between the magnet axis 2763 and the bottom surface 2754 of the array of wells. Although reference is made to the magnet 2759 moving along the array of wells, relative motion between the magnet 2759 and the array of wells can additionally or alternatively be achieved by moving the array of wells relative to the magnet, as described above.

Additionally or alternatively, the type and shape of the magnet can be selected to provide the desired magnetic field. The magnet 2759 can be a permanent magnet or an electromagnet. Any suitable magnet shape can be selected. For example and not limitation, the magnet can define a corner. Additionally or alternatively, and as embodied herein, the corner 2764 of the magnet 2759 can be in contact with the bottom surface 2754 of the array of wells as the magnet moves along the array of wells. For purpose of example and not limitation, the magnet can have a cylindrical, triangular, square, spherical, or other suitable shape. Additionally or alternatively, and as embodied herein, the magnet can have a rectangular shape.

As described above, the shape, orientation, and position of the magnetic element can be selected to provide a desired magnetic field. With reference to FIG. 35C-FIG. 35H, properties of the magnetic field in an exemplary detection region 2750 having magnet 2759, including a plurality of magnetic field lines which emit from the magnet, are depicted for purpose of illustration and explanation and not limitation. In exemplary sample detection region 2750, magnet 2759 was positioned with corner 2764 of the magnet 2759 in contact with the bottom surface 2754 of the array of wells, such that the magnetic field lines of the magnet 2759 extent through the bottom surface 2754 of the array of wells, with the magnetic field lines concentrated proximate to the corner 2764 of the magnet 2759. Additionally, exemplary magnet was positioned with magnet axis 2763 at an angle 2760 of about 20 degrees relative to the bottom surface 2754. Additionally or alternatively, exemplary magnet can be positioned with magnet axis 2763 at an angle 2760 of between about 0 degrees and 80 degrees. Additionally or alternatively, exemplary magnet can be positioned with magnet axis 2763 at an angle 2760 of between about 10 degrees and 30 degrees. With reference to FIG. 35B, an array axis “X” can be defined along top surface 2751 of the array of wells, and a second axis “Y” can be defined perpendicular to the array axis. For illustration and as embodied herein, a zero point on the array axis “X” is defined at the location on the array axis “X” where the corner 2764 of magnet 2759 contacts the bottom surface 2754 of the array of wells.

Additionally, and a zero point on the second axis “Y” is defined at the top surface 2751 of the array of wells.

With reference to FIG. 35D, magnetic force measured in pN in the array axis “X” and the second axis “Y” is shown as a function of position along the array axis “X” for the exemplary sample analysis region 2750 and magnet 2759 configuration described above. As shown, a negative magnetic force is generated in the second axis Y where the corner 2764 of magnet 2759 contacts the bottom surface 2754 of the array of wells. As embodied herein, the negative magnetic force acts on the microparticles or microparticles and assisting particles to pull the microparticles down into wells of the array of wells to load or seed the microparticles into the wells.

With reference to FIGS. 35E and 35F, magnetic force measured in pN is shown separately for the second axis “Y” and the array axis “X” as a function of position along the array axis “X” for the exemplary detection region 2750 and magnet 2759 configuration described above. As embodied herein, the magnetic force in the second axis “Y” can define a generally parabolic shape when plotted and can include a sharp negative peak in magnetic field strength where the corner 2764 of magnet 2759 contacts the bottom surface 2754 of the array of wells. Additionally, as embodied herein the magnitude of the magnetic force in the second axis “Y” can be greater than 2000 pN. As described above, the parabolic magnetic field and strong negative magnetic field can act on the microparticles to pull the microparticles into wells of the array to seed the microparticles within wells of the array. Additionally and as embodied herein, the magnetic field in the second axis “Y” can remain negative across the array axis “X” as shown. For example and as embodied herein, the magnetic field in the second axis

“Y” can remain negative over at least 6 mm as measured in the array axis “X” and as depicted in FIG. 35E. The negative magnetic field in the second axis “Y” across a wide portion of the array of wells can retain microparticles that have been seeded in wells of the array within the wells.

With reference to FIG. 35G, magnetic field strength measured in H (AIM) is shown as a function of position along the array axis “X” for the exemplary detection region 2750 and magnet 2759 configuration described above. As embodied herein, the magnet 2759 can provide a strong magnetic field with a generally parabolic shape. As embodied herein, the magnetic field strength can be about 400,000 H (AIM) where the corner 2764 of magnet 2759 contacts the bottom surface 2754 of the array of wells. The magnetic field strength can seed and retain microparticles in wells of the array of wells as described herein.

With reference to FIG. 35H, magnetic force measured in pN in the array axis “X” (Fx) and in the second axis “Y” (Fy) is shown as a function of position along the array axis “X” and along the second axis “Y” for the exemplary detection region 2750 and magnet 2759 configuration described above.

With continued reference to FIGS. 35B-35E, the magnetic field achieved can provide benefits for seeding and sealing microparticles into wells. For example and not limitation, the magnitude of the y-component of magnetic force relative to the x-component is high at the location where the corner 2764 of magnet 2759 contacts the bottom surface 2754 of the array of wells. The relatively high magnitude of the y-component can help to ensure microparticles are loaded (i.e., seeded) into wells and remain in wells during the sealing process. Additionally, the magnitude of the magnetic field is high, and the distribution of the magnetic field has a high peak. Having a distribution of magnetic field with a high peak can facilitate aggregation of microparticles or microparticles and assisting particles as the microparticles or microparticles and assisting particles are moved across the array of wells. Increased aggregation of microparticles or microparticles and assisting particles during movement across the array of wells can result in fewer excess microparticles left on the top of the array. Additionally, the magnitude of the x-component of magnetic force can be large enough to continuously pull excess microparticles along the surface of the array of wells. The increased magnitude of the magnetic force in the array axis “X” can help to further remove excess microparticles left on the top of array.

Assays that may be performed using the exemplary sample paths discussed above are described further in section for methods of detecting a target analyte in a sample.

### II. Benefits of the Devices

The devices of the present disclosure provide certain benefits over devices known in the art. The benefits of the devices of the present disclosure include high levels of modularity, the ability to use small volumes, and provide the ability to analyze multiple sample types simultaneously. In terms of the modularity, the multiple exemplary embodiments of the devices disclosed herein provide high levels of configurability through the usage of multiple primary zones for sample or reagent addition, multiple secondary zones for hydrophobic separation (e.g., through the presence of air) or for merging to create large sample or reagent zones, many different types of sample analysis regions configured to analyze proteins or antigens (erg, immunoassays and clinical chemistry), nucleic acids (e.g., nucleic acid analysis), metabolites (e.g., clinical chemistry), or cells or cells types (e.g., immunoassays, clinical chemistry, nucleic acids, or CBC). The primary and secondary zones can be configured by the user to fit any type of assay or the particular steps of a given assay. The ability to perform these assays using small volumes allows for reduced costs of reagents and reduced consumption of sample which allows for the conservation of difficult to obtain samples. The modularity of the device also allows assays to be customized to the user's preference and also allows multiple assays (e.g., immunoassays and nucleic acid analysis, or any combination of the assays disclosed above) to be performed simultaneously or sequentially on the same device for the same sample or multiple samples contained on a single device.

### 2. Device Design

The devices of the present disclosure have a range of different layouts with varying numbers of primary and secondary zones, different arrangements of primary and secondary zones, the presence or absence of sample mixing zones, and the presence or absence of fixed primary zones. Exemplary embodiments of the devices are disclosed in FIGS. 1-9, 11-19 and 32.

FIG. 1 discloses an illustration of an embodiment of the device. In this embodiment, the device 100 contains a 3×8 grid of primary zones 101 and secondary zones 107. The device comprises a sample processing region 110. The sample processing region 110 is unbounded. By unbounded it is meant that there is no physical barrier separating the primary zones 101 and the secondary zones 107. The sample processing region comprises a primary zone 101 that has an opening 103 in the primary zone. The primary zone is adjacent to the secondary zone 107. The secondary zone may comprise an opening 104. The sample processing region 110 may be connected to a tertiary zone (i.e., the sample detection zone) 112 by a transition zone 113. The device may further comprise a quaternary zone 114 (i.e., a hydrophilic liquid well) that is connected to the tertiary zone. The device may further comprise a quinary zone 115 (i.e., a hydrophobic liquid well) that is connected to the tertiary zone. The top and the bottom substrate are bound by adhesive or clips 111. In some embodiments, the top substrate and bottom are bound by an adhesive layer between the top and bottom substrate. In some embodiments, the top and bottom substrate are bound using laser welding.

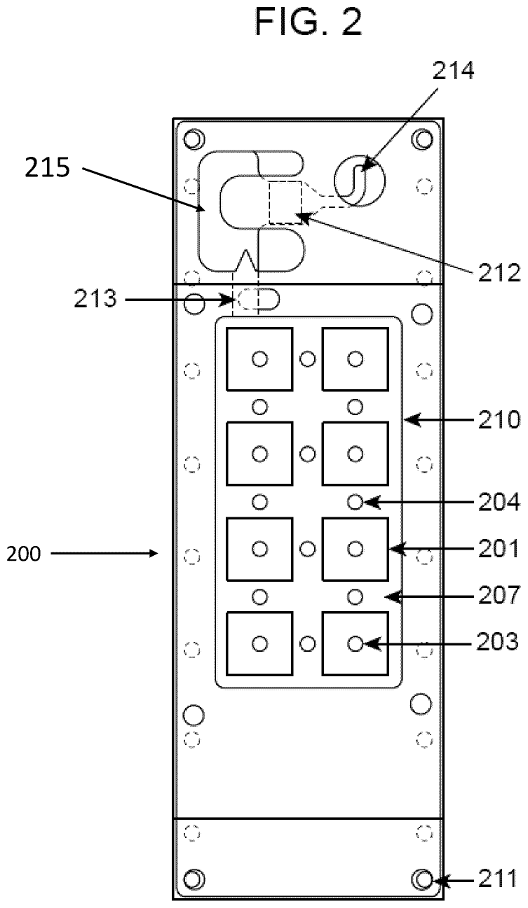

An alternative embodiment to FIG. 1 is disclosed below. In this embodiment, the device 100 contains a 3×8 grid of primary zones 101 and secondary zones 107. The device 100 comprises a second substrate positioned on a first substrate. The second substrate comprises a surface facing a central chamber comprising a plurality of recessed elements, and a plurality of protruding elements. The primary zones 101 are defined between a surface of the plurality of protruding elements facing the first substrate and a surface of the first substrate facing the second substrate. The secondary zones are defined between a surface of the plurality of recessed elements facing the first substrate and the surface of the first substrate facing the second substrate. The device comprises a sample processing region 110. The sample processing region 110 is unbounded. By unbounded it is meant that there is no physical barrier separating the primary zones 101 and the secondary zones 107. While there is only a single element label for the primary zone 101, each square (101) is to represent an individual primary zone. In some embodiments, the primary zones are discrete. While there is only a single element label for the secondary zone 107, each space between the squares is to represent an individual secondary zone. In some embodiments, the secondary zones are connected. The sample processing region comprises a primary zone 101 that has an opening 103 in the primary zone. While there is only a single element label for the opening in the primary zone 103, each opening (103) in the square (101) is meant to represent an individual opening in the primary zone. The primary zone is adjacent to the secondary zone 107. The secondary zone may comprise an opening 104. While there is only a single element label for the opening in the secondary zone 107, each opening (104) in between the squares represents an individual opening in the secondary zone. The sample processing region 110 may be positioned adjacent to a sample analysis region comprising a tertiary zone (i.e., the sample detection zone) 112, a quaternary zone 114 (i.e., a hydrophilic liquid well), and a quinary zone 115 (i.e., a hydrophobic liquid well) wherein the tertiary zone 112, the quaternary zone 114, and the quinary zone 115 are connected. The sample analysis region is defined by the surface of the second substrate facing the first substrate and the surface of the first substrate facing the second substrate. The quinary zone 115 is located at a first end of the sample analysis region. The quinary zone comprises an opening spanning the second substrate The quaternary zone 114 is located at a second end of the sample analysis region. The quaternary zone comprises a cylindrical opening spanning the second substrate. The tertiary zone 112 is located at a midpoint between the first end (e.g., the quinary zone 115) and the second end (e.g., the quaternary zone 115) of the sample analysis region. The sample analysis region may be connected to the sample processing region 110 by a transition zone 113. The second substrate positioned over the first substrate are bound together by adhesive or clips 111. In some embodiments, the second substrate and the first substrate are bound by an adhesive layer between the second substrate and the first substrate. In some embodiments, the second substrate and the first substrate are bound using laser welding. The alternative embodiment disclosed for FIG. 1 may be applied to FIG. 2-6 such that the descriptions above describe the elements of the devices disclosed in FIG. 2-6.

FIG. 2 discloses an illustration of an embodiment of the device. In this embodiment, the device 200 contains a 2×4 grid of primary zones 201 and secondary zones 207. The device comprises a sample processing region 210. The sample processing region 210 is unbounded. By unbounded it is meant that there is no physical barrier separating the primary zones 201 and the secondary zones 207. The sample processing region comprises a primary zone 201 that has an opening 203 in the primary zone. The primary zone is adjacent to the secondary zone 207. The secondary zone may comprise an opening 204. The sample processing region 210 may be connected to a tertiary zone (i.e., the sample detection zone) 212 by a transition zone 213. The device may further comprise a quaternary zone 214 (i.e., a hydrophilic liquid well) that is connected to the tertiary zone. The device may further comprise a quinary zone 215 (i.e., a hydrophobic liquid well) that is connected to the tertiary zone. The top and the bottom substrate are bound by adhesive or clips 211. In some embodiments, the top substrate and bottom are bound by an adhesive layer between the top and bottom substrate. In some embodiments, the top and bottom substrate are bound using laser welding.

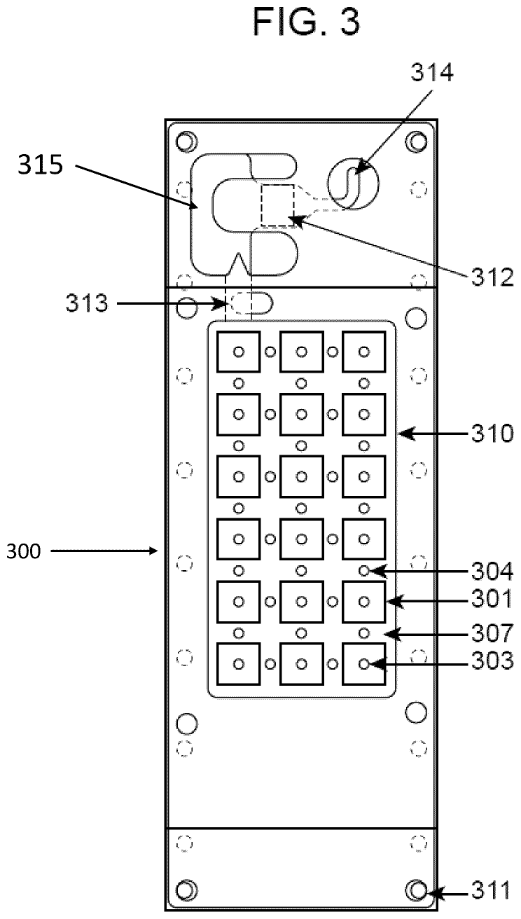

FIG. 3 discloses an illustration of an embodiment of the device. In this embodiment, the device 300 contains a 3×6 grid of primary zones 301 and secondary zones 307. The device comprises a sample processing region 310. The sample processing region 310 is unbounded. By unbounded it is meant that there is no physical barrier separating the primary zones 301 and the secondary zones 307. The sample processing region comprises a primary zone 301 that has an opening 303 in the primary zone. The primary zone is adjacent to the secondary zone 307. The secondary zone may comprise an opening 304. The sample processing region 310 may be connected to a tertiary zone (i.e., the sample detection zone) 312 by a transition zone 313. The device may further comprise a quaternary zone 314 (i.e., a hydrophilic liquid well) that is connected to the tertiary zone. The device may further comprise a quinary zone 315 (i.e., a hydrophobic liquid well) that is connected to the tertiary zone. The top and the bottom substrate are bound by adhesive or clips 311. In some embodiments, the top substrate and bottom are bound by an adhesive layer between the top and bottom substrate. In some embodiments, the top and bottom substrate are bound using laser welding.

FIG. 3 discloses an illustration of an embodiment of the device. In this embodiment, the device 300 contains a 3×6 grid of primary zones 301 and secondary zones 307. The device comprises a sample processing region 310. The sample processing region 310 is unbounded. By unbounded it is meant that there is no physical barrier separating the primary zones 301 and the secondary zones 307. The sample processing region comprises a primary zone 301 that has an opening 303 in the primary zone. The primary zone is adjacent to the secondary zone 307. The secondary zone may comprise an opening 304. The sample processing region 310 may be connected to a tertiary zone (i.e., the sample detection zone) 312 by a transition zone 313. The device may further comprise a quaternary zone 314 (i.e., a hydrophilic liquid well) that is connected to the tertiary zone. The device may further comprise a quinary zone 315 (i.e., a hydrophobic liquid well) that is connected to the tertiary zone. The top and the bottom substrate are bound by adhesive or clips 311. In some embodiments, the top substrate and bottom are bound by an adhesive layer between the top and bottom substrate. In some embodiments, the top and bottom substrate are bound using laser welding.

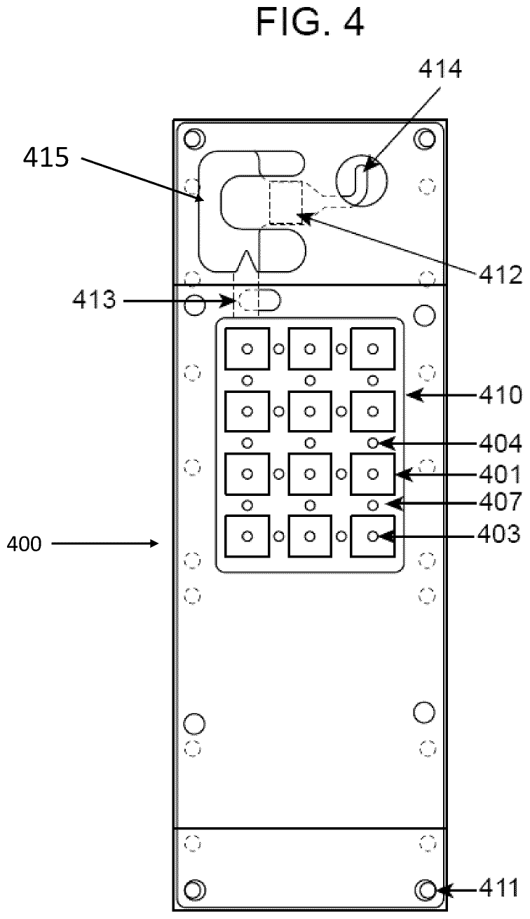

FIG. 4 discloses an illustration of an embodiment of the device. In this embodiment, the device 400 contains a 3×4 grid of primary zones 401 and secondary zones 407. The device comprises a sample processing region 410. The sample processing region 410 is unbounded. By unbounded it is meant that there is no physical barrier separating the primary zones 401 and the secondary zones 407. The sample processing region comprises a primary zone 401 that has an opening 403 in the primary zone. The primary zone is adjacent to the secondary zone 407. The secondary zone may comprise an opening 404. The sample processing region 410 may be connected to a tertiary zone (i.e., the sample detection zone) 412 by a transition zone 413. The device may further comprise a quaternary zone 414 (i.e., a hydrophilic liquid well) that is connected to the tertiary zone. The device may further comprise a quinary zone 415 (i.e., a hydrophobic liquid well) that is connected to the tertiary zone. The top and the bottom substrate are bound by adhesive or clips 411. In some embodiments, the top substrate and bottom are bound by an adhesive layer between the top and bottom substrate. In some embodiments, the top and bottom substrate are bound using laser welding.

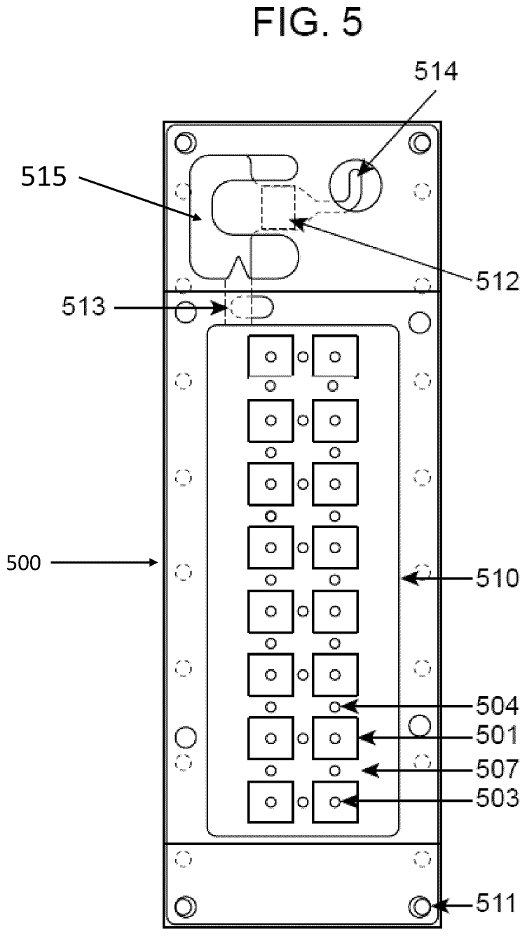

FIG. 5 discloses an illustration of an embodiment of the device. In this embodiment, the device 500 contains a 2×8 grid of primary zones 501 and secondary zones 507. The device comprises a sample processing region 510. The sample processing region 510 is unbounded. By unbounded it is meant that there is no physical barrier separating the primary zones 501 and the secondary zones 507. The sample processing region comprises a primary zone 501 that has an opening 503 in the primary zone. The primary zone is adjacent to the secondary zone 507. The secondary zone may comprise an opening 504. The sample processing region 510 may be connected to a tertiary zone (i.e., the sample detection zone) 512 by a transition zone 513. The device may further comprise a quaternary zone 514 (i.e., a hydrophilic liquid well) that is connected to the tertiary zone. The device may further comprise a quinary zone 515 (i.e., a hydrophobic liquid well) that is connected to the tertiary zone. The top and the bottom substrate are bound by adhesive or clips 511. In some embodiments, the top substrate and bottom are bound by an adhesive layer between the top and bottom substrate. In some embodiments, the top and bottom substrate are bound using laser welding.

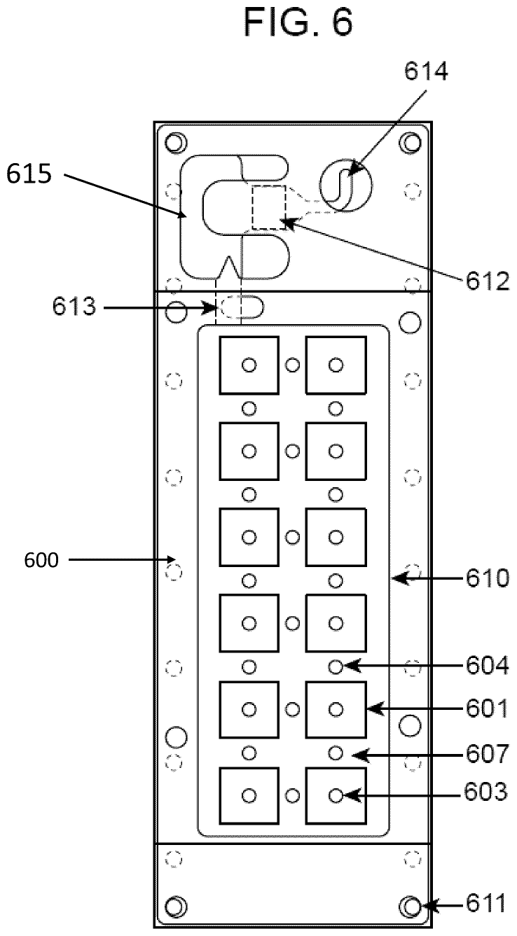

FIG. 6 discloses an illustration of an embodiment of the device. In this embodiment, the device 600 contains a 2×6 grid of primary zones 601 and secondary zones 607. The device comprises a sample processing region 610. The sample processing region 610 is unbounded. By unbounded it is meant that there is no physical barrier separating the primary zones 601 and the secondary zones 607. The sample processing region comprises a primary zone 601 that has an opening 603 in the primary zone. The primary zone is adjacent to the secondary zone 607. The secondary zone may comprise an opening 604. The sample processing region 610 may be connected to a tertiary zone (i.e., the sample detection zone) 612 by a transition zone 613. The device may further comprise a quaternary zone 614 (i.e., a hydrophilic liquid well) that is connected to the tertiary zone. The device may further comprise a quinary zone 615 (i.e., a hydrophobic liquid well) that is connected to the tertiary zone. The top and the bottom substrate are bound by adhesive or clips 611. In some embodiments, the top substrate and bottom are bound by an adhesive layer between the top and bottom substrate. In some embodiments, the top and bottom substrate are bound using laser welding.

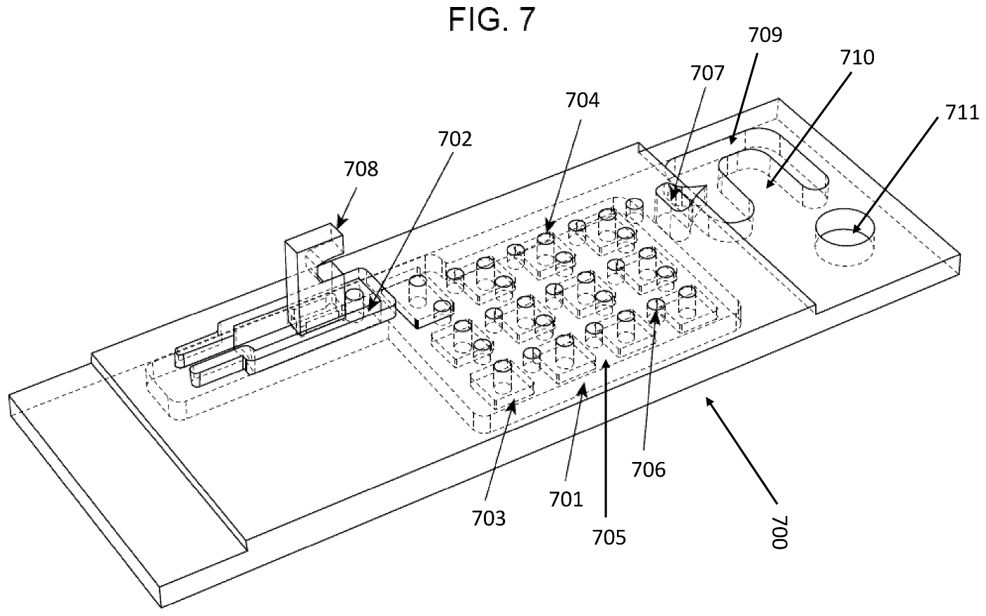

FIG. 7 discloses an illustration of an isometric view of an embodiment of the device. The dotted lines represent interior portions of the device whereas solid lines represent exterior portions of the device. In this embodiment, the device 700 contains a 3×4 grid of primary zones 703 and secondary zones 705 in addition to a fixed primary zone 702. In some instances, but not all instances, the fixed primary zone 702 is also a sample mixing zone. The fixed primary zone is physically connected to a hooked portion 708. The boundaries of the top outside portion of the fixed primary zone is not attached to the rest of the device except for the portion furthest from the primary and secondary zones. When a vertical force is applied to the hooked portion 708, the top substrate (i.e., the roof) compresses and decompresses fluid (e.g., sample) that may be in the fixed primary zone 702 thereby mixing the fluid. The device comprises a sample processing region 701. The sample processing region 701 is unbounded. By unbounded it is meant that there is no physical barrier separating the primary zones 703 and the secondary zones 705. The sample processing region comprises a primary zone 703 that has an opening 704 in the primary zone. The primary zone is adjacent to a secondary zone such as 705. The secondary zone may comprise an opening 706. The sample processing region 701 be connected to a tertiary zone (i.e. the sample detection zone) 710 by a transition zone 707. The device may further comprise a quaternary zone 711 (i.e., a hydrophilic liquid well) that is connected to the tertiary zone. The device may further comprise a quinary zone 709 (i.e., a hydrophobic liquid well) that is connected to the tertiary zone.

An alternative embodiment to FIG. 7 is disclosed below. The dotted lines represent interior portions of the device whereas solid lines represent exterior portions of the device. In this embodiment, the device 700 contains a 3×4 grid of primary zones 703 and secondary zones 705 in addition to a fixed primary zone 702. The device 700 comprises a second substrate positioned on a first substrate. The second substrate comprises a surface facing a central chamber comprising a plurality of recessed elements, and a plurality of protruding elements. The primary zones 701 are defined between a surface of the plurality of protruding elements facing the first substrate and a surface of the first substrate facing the second substrate. The secondary zones are defined between a surface of the plurality of recessed elements facing the first substrate and the surface of the first substrate facing the second substrate. In some instances, but not all instances, the fixed primary zone 702 is also a sample mixing zone. The fixed primary zone 702 is formed from a laterally extended protruding element in the second substrate facing the first substrate wherein the laterally extended protruding element has a surface facing the first substrate that has a greater surface area than the surface of the plurality of protruding elements facing the first substrate. The fixed primary zone 707 comprises hooked portion 708 joined to a surface of the extended protruding element opposite the surface of the second substrate facing the first substrate. The boundaries of the top outside portion of the fixed primary zone are not attached to the rest of the device except for the portion furthest from the primary and secondary zones. When a vertical force is applied to the hooked portion 708, the second substrate (i.e., the roof) compresses and decompresses fluid (e.g., sample) that may be in the fixed primary zone 702 thereby mixing the fluid. The device comprises a sample processing region 701. The sample processing region 701 is unbounded. By unbounded it is meant that there is no physical barrier separating the primary zones 703 and the secondary zones 705. The sample processing region comprises a primary zone 703 that has an opening 704 in the primary zone. The primary zone is adjacent to a secondary zone such as 705. The secondary zone may comprise an opening 706. The sample processing region 701 may be positioned adjacent to a sample analysis region comprising a tertiary zone (i.e., the sample detection zone) 710, a quaternary zone 711 (i.e., a hydrophilic liquid well), and a quinary zone 709 (i.e., a hydrophobic liquid well) wherein the tertiary zone 707, the quaternary zone 711, and the quinary zone 709 are connected. The sample analysis region is defined by the surface of the second substrate facing the first substrate and the surface of the first substrate facing the second substrate. The quinary zone 709 is located at a first end of the sample analysis region. The quinary zone comprises an opening spanning the second substrate. The quaternary zone 711 is located at a second end of the sample analysis region. The quaternary zone comprises a cylindrical opening spanning the second substrate. The tertiary zone 710 is located at a midpoint between the first end (e.g., the quinary zone 709) and the second end (e.g., the quaternary zone 711) of the of the sample analysis region. The sample analysis region may be connected to the sample processing region 701 by a transition zone 707. The alternative embodiment disclosed for FIG. 7 may be applied to FIG. 15 such that the descriptions above describe the elements of the devices disclosed in FIG. 15.

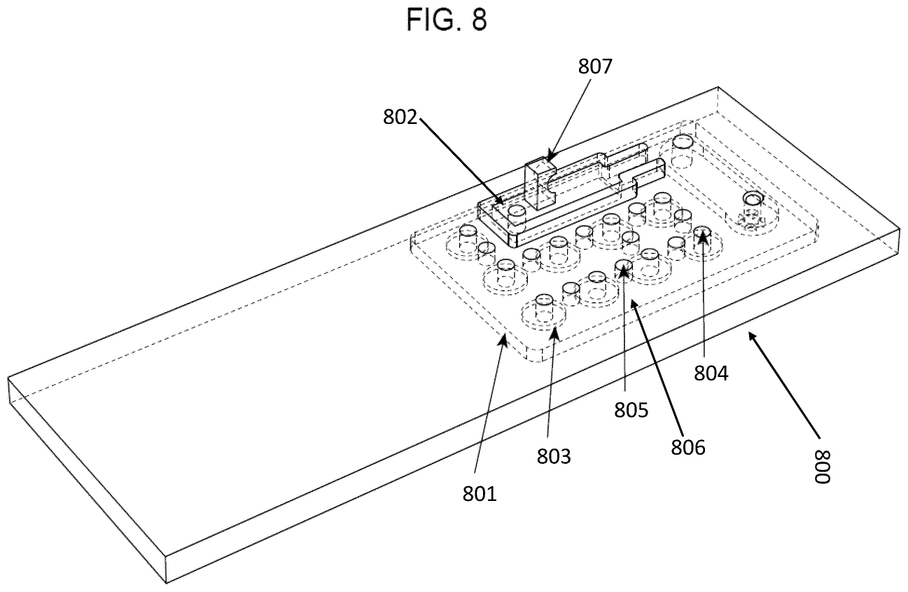

FIG. 8 discloses an illustration of an isometric view of an embodiment of the device. The dotted lines represent interior portions of the device whereas solid lines represent exterior portions of the device. In this embodiment, the device 800 contains a 2×4 grid of primary zones 803 and secondary zones 806 with in addition to one additional primary zone and a fixed primary zone 802. In some instances, but not all instances, the fixed primary zone 802 is also a sample mixing zone. The fixed primary zone is physically connected to a hooked portion 807. The boundaries of the top outside portion of the fixed primary zone is not attached to the rest of the device except for the portion furthest from the primary and secondary zones. When a vertical force is applied to the hooked portion 807, the top substrate (i.e., the roof) compresses and decompresses fluid (e.g., sample) that may be in the fixed primary zone 802 thereby mixing the fluid. The device comprises a sample processing region 801. The sample processing region 801 is unbounded. By unbounded it is meant that there is no physical barrier separating the primary zones 803 and the secondary zones 806. The sample processing region comprises a primary zone 803 that has an opening 804 in the primary zone. The primary zone is adjacent to a secondary zone such as 806. The secondary zone may comprise an opening 805.

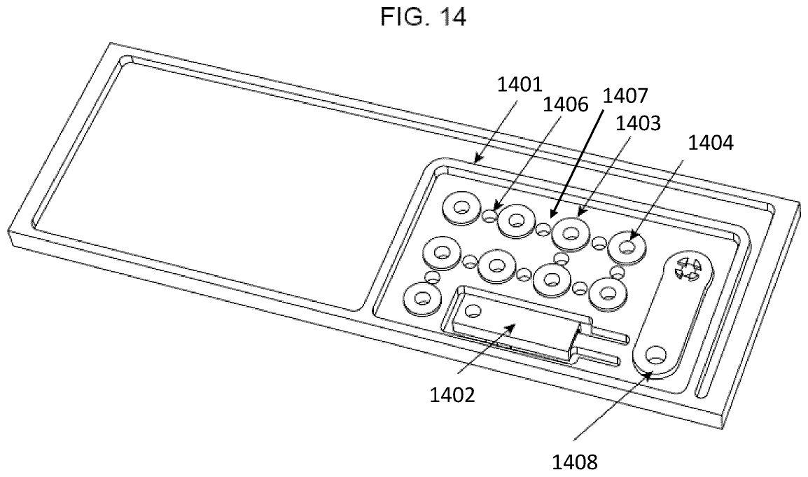

An alternative embodiment to FIG. 8 is disclosed below. The dotted lines represent interior portions of the device whereas solid lines represent exterior portions of the device. In this embodiment, the device 800 contains a 2×4 grid of primary zones 803 and secondary zones 806 with in addition to one additional primary zone and a fixed primary zone 802. The device 800 comprises a second substrate positioned on a first substrate. The second substrate comprises a surface facing a central chamber comprising a plurality of recessed elements, and a plurality of protruding elements. The primary zones are defined between a surface of the plurality of protruding elements facing the first substrate and a surface of the first substrate facing the second substrate. The secondary zones are defined between a surface of the plurality of recessed elements facing the first substrate and the surface of the first substrate facing the second substrate. In some instances, but not all instances, the fixed primary zone 802 is also a sample mixing zone. The fixed primary zone 802 is formed from a laterally extended protruding element in the second substrate facing the first substrate wherein the laterally extended protruding element has a surface facing the first substrate that has a greater surface area than the surface of the plurality of protruding elements facing the first substrate. The fixed primary zone 802 comprises hooked portion 807 joined to a surface of an extended protruding element opposite the surface of the second substrate facing the first substrate. The boundaries of the top outside portion of the fixed primary zone is not attached to the rest of the device except for the portion furthest from the primary and secondary zones. When a vertical force is applied to the hooked portion 807, the second substrate (i.e., the roof) compresses and decompresses fluid (e.g., sample) that may be in the fixed primary zone 802 thereby mixing the fluid. The device comprises a sample processing region 801. The sample processing region 801 is unbounded. By unbounded it is meant that there is no physical barrier separating the primary zones 803 and the secondary zones 806. The sample processing region comprises a primary zone 803 that has an opening 804 in the primary zone. The primary zone is adjacent to a secondary zone such as 806. The secondary zone may comprise an opening 805. The alternative embodiment disclosed for FIG. 8 may be applied to FIG. 14 such that the descriptions above describe the elements of the devices disclosed in FIG. 14.

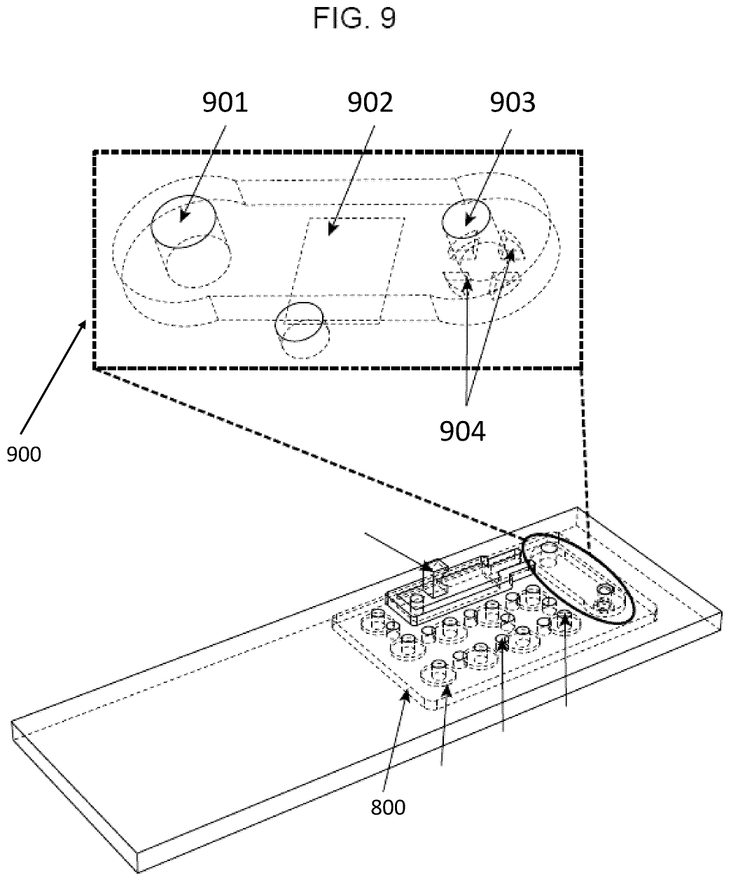

FIG. 9 discloses an illustration of an exemplary sample analysis region of the embodiment depicted in FIG. 8. The sample processing region 800 may be connected to a sample analysis region 900 comprising a tertiary zone (i.e., the sample detection zone) 902. The device may further comprise a quaternary zone 903 (i.e., a hydrophilic liquid well) that is connected to the tertiary zone. The quaternary zone 903 (i.e., a hydrophilic liquid well) may be surrounded by substrate retention features 904. The device may further comprise a quinary zone 901 (i.e., a hydrophobic liquid well) that is connected to the tertiary zone. The sample analysis region may be a pad such as those in the primary zones. In some embodiments, when the sample analysis region is a pad, the pad has the same thickness as the pads in the primary zones. In some embodiments, when the sample analysis region is a pad, the pad has a different thickness as the pads in the primary zones. A substrate retention feature may be configured to assist in the removal of the hydrophilic liquids disclosed herein.

An alternative embodiment to FIG. 9 is disclosed below. The sample processing region 800 may be connected to a sample analysis region 900 comprising a tertiary zone (i.e., the sample detection zone) 902. The sample analysis region comprises an enlarged protruding element where a surface of the enlarged protruding element facing the first substrate has a greater surface area than a surface of the plurality of protruding elements facing the first substrate, and the enlarged protruding element extends from the first end (e.g., a quinary zone 901) to the second end (e.g., a quaternary zone 903). The device may further comprise a quaternary zone 903 (i.e., a hydrophilic liquid well) that is connected to the tertiary zone. The quaternary zone 903 (i.e., a hydrophilic liquid well) may be surrounded by substrate retention features 904. The quaternary zone comprises a cylindrical opening spanning the second substrate. The device may further comprise a quinary zone 901 (i.e., a hydrophobic liquid well) that is connected to the tertiary zone. The quinary zone comprises an opening spanning the second substrate. The sample analysis region 900 is adjacent to the sample processing region 800. The sample analysis region is not physically separated from the sample processing region.

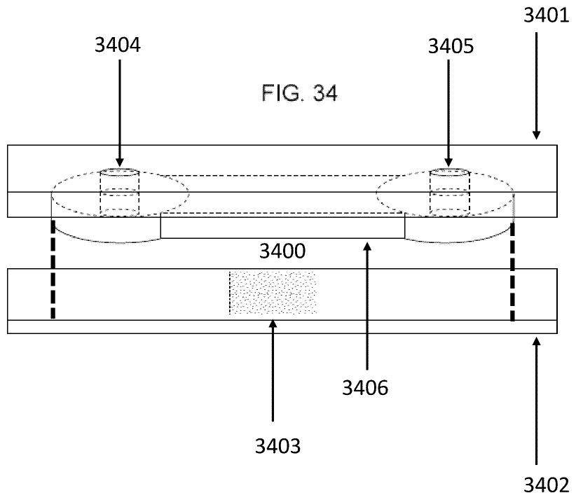

FIG. 34 discloses an illustration of an exemplary sample analysis region of the embodiment depicted in FIG. 8 and FIG. 9. The thin dotted lines represent interior portions of the device whereas solid lines represent exterior portions of the device. The sample analysis region 3400 (bounded by thick dashed lines) is formed by the top substrate 3401 bound to the bottom substrate 3402. A pad 3406 defines the sample analysis region. The sample analysis region 3400 comprises a tertiary zone 3402 (i.e., sample detection zone). The device may further comprise a quaternary zone 3405 (i.e., a hydrophilic liquid well) that is connected to the tertiary zone. By connected is meant that the zones are adjacent to each other. The quaternary zone 3405 (i.e., a hydrophilic liquid well) may be surrounded by substrate retention regions (not shown; as depicted in FIG. 9 904). The device may further comprise a quinary zone 3404 (i.e., a hydrophobic liquid well) that is connected to the tertiary zone.

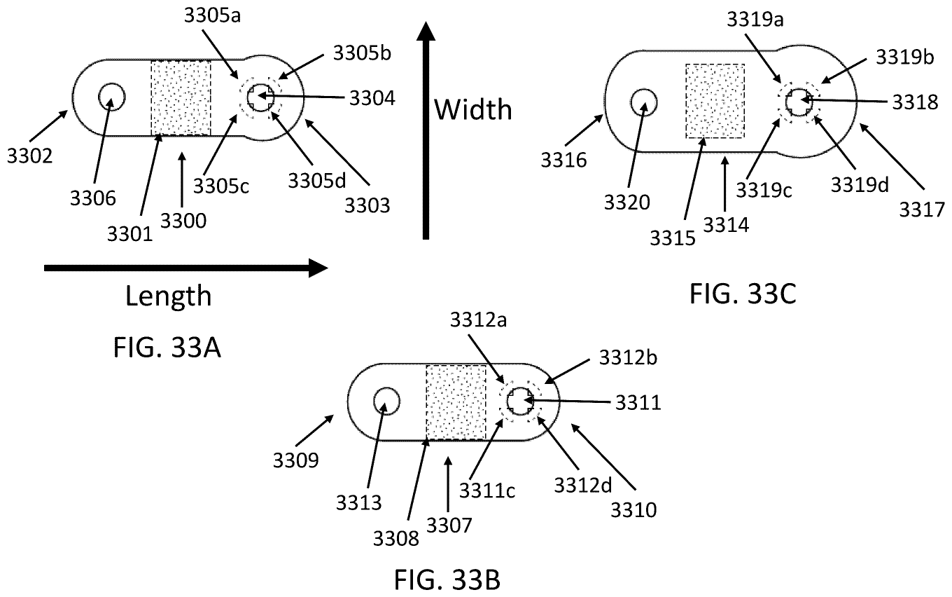

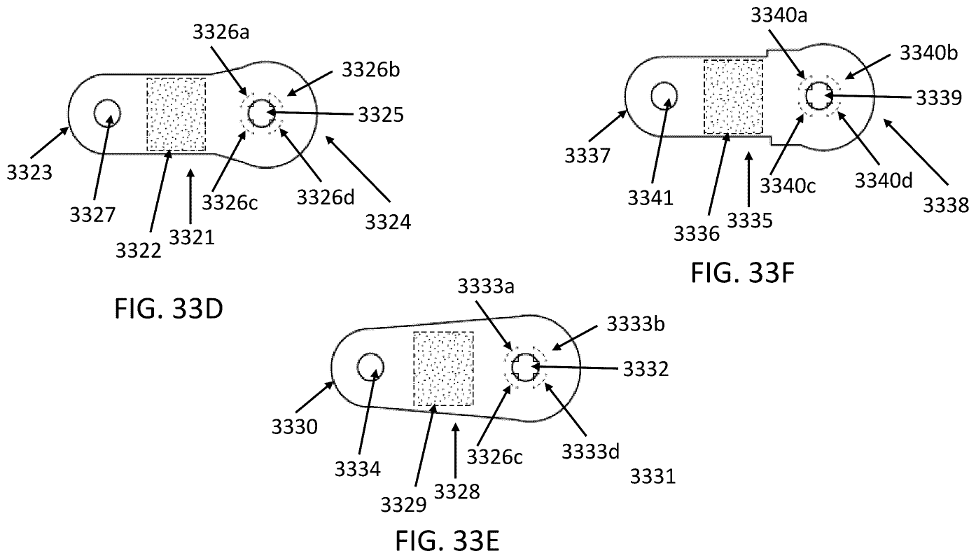

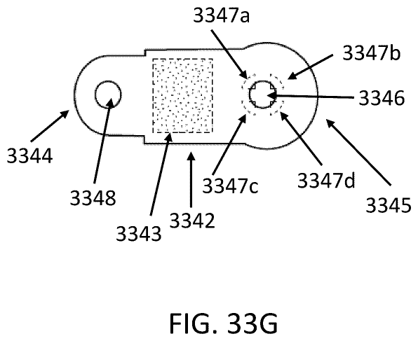

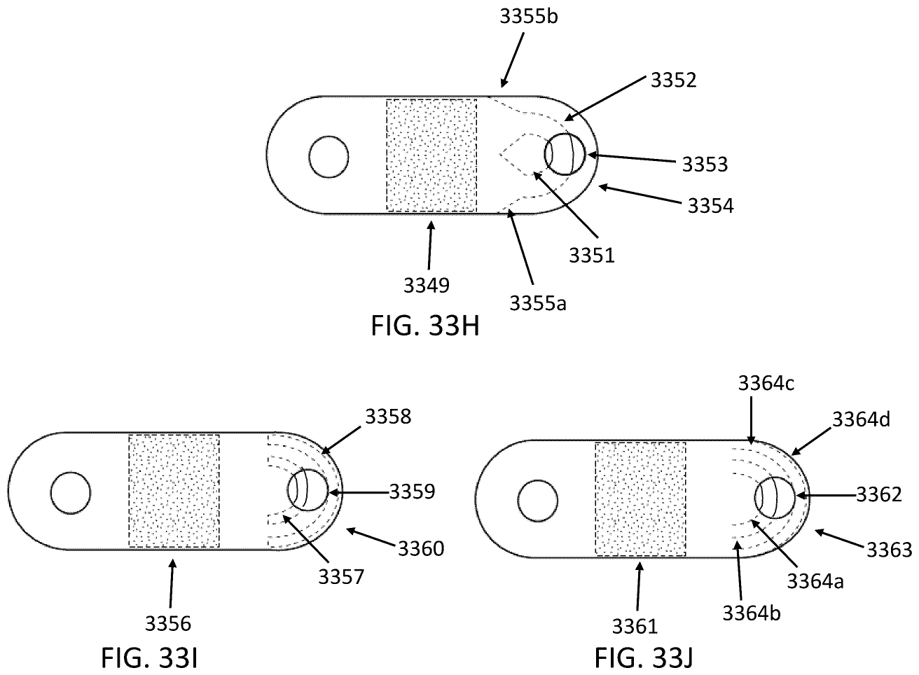

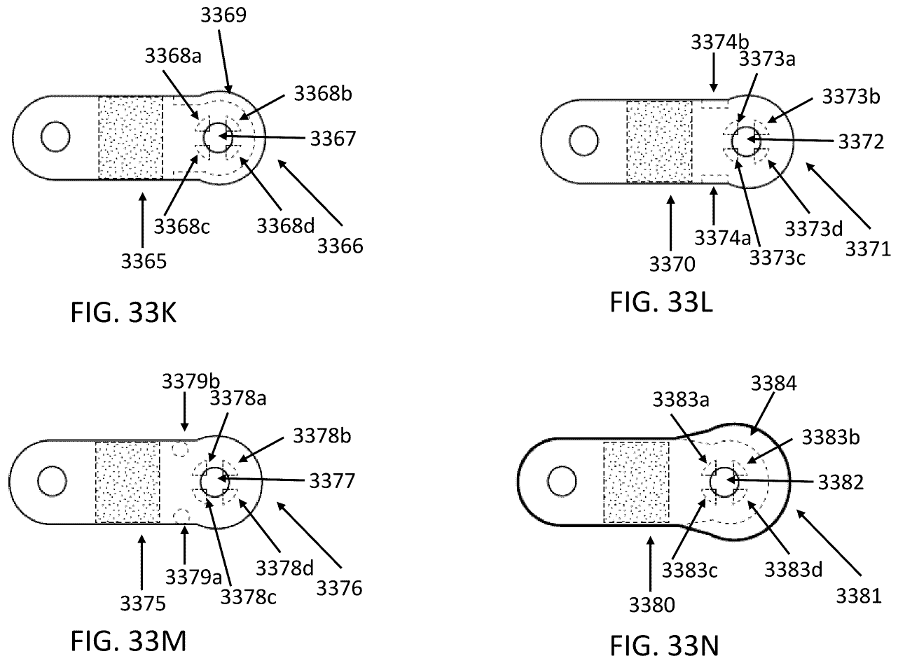

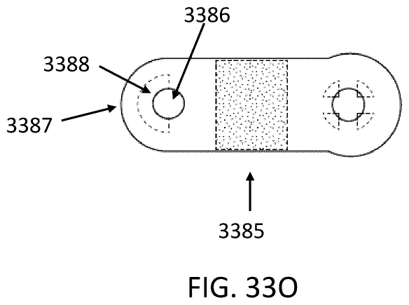

FIG. 33A-G discloses illustrations of exemplary sample analysis regions. FIG. 33A discloses an illustration of the sample analysis regions depicted in FIGS. 8 and 9. The dotted lines represent interior portions of the device whereas solid lines represent exterior portions of the device. The sample analysis region 3300 comprises a tertiary zone 3301 (e.g., a sample detection zone) that is the width of the sample analysis region 3300. The sample analysis region 3300 comprises a quaternary zone 3304 (i.e., hydrophilic liquid well) that has substrate retention features 3305a-d which are quarter circle shaped and protrude into the quaternary zone 3304. The sample analysis region comprises a quinary zone 3306 (e.g., a hydrophobic liquid well). The left end 3302 has an oval shape that has a smaller area than the right end 3303 which has a larger area and is a circular shape.

FIG. 33B discloses an illustration of an exemplary sample analysis region. The sample analysis region 3307 comprises a tertiary zone 3308 (e.g., a sample detection zone) that is the width of the sample analysis region 3307. The sample analysis region 3307 comprises a quaternary zone 3311 (i.e., hydrophilic liquid well) that has substrate retention features 3312a-d which are quarter circle shaped and protrude into the quaternary zone 3311. The sample analysis region comprises a quinary zone 3313 (e.g., a hydrophobic liquid well). The left end 3309 has an oval shape and the right end 3310 has an oval shape that is the same size as the left end.

FIG. 33C discloses an illustration of an exemplary sample analysis region. The sample analysis region 3314 comprises a tertiary zone 3315 (e.g., a sample detection zone) where the width of the sample analysis region is larger than the tertiary zone. The sample analysis region 3314 comprises a quaternary zone 3318 (i.e., hydrophilic liquid well) that has substrate retention features 3319a-d which are quarter circle shaped and protrude into the quaternary zone 3318. The sample analysis region comprises a quinary zone 3320 (e.g., a hydrophobic liquid well). The left end 3316 has an oval shape that has a smaller area than the right end 3317 which has a larger area and is a circular shape.

FIG. 33D discloses an illustration of an exemplary sample analysis region. The sample analysis region 3321 comprises a tertiary zone 3322 (e.g., a sample detection zone) that is the width of the sample analysis region 3321. The sample analysis region 3321 comprises a quaternary zone 3325 (i.e., hydrophilic liquid well) that has substrate retention features 3326a-d which are quarter circle shaped and protrude into the quaternary zone 325. The sample analysis region comprises a quinary zone 3327 (e.g., a hydrophobic liquid well). The left end 3323 has an oval shape that has a smaller area than the right end 3324 which has a larger area and is a circular shape.

FIG. 33E discloses an illustration of an exemplary sample analysis region. The sample analysis region 3328 comprises a tertiary zone 3329 (e.g., a sample detection zone) wherein the width of the sample analysis region 3321 is smaller on the edge of the tertiary zone 3329 closest to the left end 3330 than on the edge of the tertiary zone 3329 closest to the left end 3331. The sample analysis region 3328 comprises a quaternary zone 3332 (i.e., hydrophilic liquid well) that has substrate retention features 3333a-d which are quarter circle shaped and protrude into the quaternary zone 3332. The sample analysis region comprises a quinary zone 3334 (e.g., a hydrophobic liquid well). The left end 3330 has an oval shape that has a smaller area than the right end 3331 which has a larger area and is an oval shape.

FIG. 33F discloses an illustration of an exemplary sample analysis region. The sample analysis region 3335 comprises a tertiary zone 3336 (e.g., a sample detection zone) that is the width of the sample analysis region 3335. The sample analysis region 3335 comprises a quaternary zone 3339 (i.e., hydrophilic liquid well) that has substrate retention features 3340a-d which are quarter circle shaped and protrude into the quaternary zone 3339. The sample analysis region comprises a quinary zone 3341 (e.g., a hydrophobic liquid well). The left end 3337 has an oval shape that has a smaller area than the right end 3338 which has a larger area and is a circular shape with a step reduction in the width prior to the tertiary zone 3336. By “step reduction in width” it is meant that there is a symmetrical reduction in the width that is immediate and not gradual.

FIG. 33G discloses an illustration of an exemplary sample analysis region. The sample analysis region 3342 comprises a tertiary zone 3343 (e.g., a sample detection zone) where the width of the sample analysis region is larger than the tertiary zone. The sample analysis region 3342 comprises a quaternary zone 3346 (i.e., hydrophilic liquid well) that has substrate retention features 3347a-d which are quarter circle shaped and protrude into the quaternary zone 3346. The sample analysis region comprises a quinary zone 3348 (e.g., a hydrophobic liquid well). The left end 3344 has an oval shape that has a smaller area than the right end 3345 which has a larger area and is a circular shape. The left end has a step reduction in the width prior to the tertiary zone 3343.

FIG. 33H-N discloses illustrations of exemplary substrate retention features. In FIG. 33H, the sample analysis region 3349 comprises a quaternary zone 3351 (i.e., hydrophilic liquid well) that has substrate retention features 3351 and 3352. The substrate retention features 3351 and 3352 may protrude from the bottom substrate to the top substrate or from the top substrate to the bottom substrate. The substrate retention feature 3351 is centrally located on the right end 3354 and is quarter circle shaped. The substrate retention feature 3352 is located on the right end 3354 of the sample analysis region 3349 and is horseshoe shaped with tapered edges. The substrate retention features of FIG. 33H may be applied to any sample analysis region disclosed herein.

In FIG. 33I, the sample analysis region 3356 comprises a quaternary zone 3359 (i.e., hydrophilic liquid well) that has substrate retention features 3357 and 3358. The substrate retention features 3357 and 3358 may protrude from the bottom substrate to the top substrate or from the top substrate to the bottom substrate. The substrate retention feature 3357 is centrally located on the right end 3360 and is horseshoe shaped without tapered edges. The substrate retention feature 3358 is located on the right end 3360 of the sample analysis region 3356 and is horseshoe shaped without tapered edges. The substrate retention features 3357 and 3358 are concentric such that the substrate retention feature 3357 is nested in the substrate retention feature 3358. The substrate retention features of FIG. 33I may be applied to any sample analysis region disclosed herein.

In FIG. 33J, the sample analysis region 3361 comprises a quaternary zone 3362 (i.e., hydrophilic liquid well) that has substrate retention features 3364a-d. The substrate retention features 3364a-d are grooves in either the top substrate or bottom substrate of the sample analysis region 3361. The substrate retention features 3364a-d are located on the right end 3363 of the sample analysis region 3361 and is horseshoe shaped. The substrate retention features 3364a-d are concentric such that the substrate retention feature 3364a is nested in the substrate retention feature 3364b, substrate retention feature 3364b is nested in the substrate retention feature 3364c, and substrate retention feature 3364c is nested in the substrate retention feature 3364d. The substrate retention features of FIG. 33I may be applied to any sample analysis region disclosed herein.

In FIG. 33K, the sample analysis region 3365 comprises a quaternary zone 3367 (i.e., hydrophilic liquid well) that has substrate retention features 3368a-d and 3369. The substrate retention features 3368a-d are quarter circle shaped and protrude into the quaternary zone 3367 and are located on the right end 3366. The substrate retention feature 3369 is located on the right end 3366 of the sample analysis region 3365 and is horseshoe shaped. The substrate retention feature 3369 may protrude from the bottom substrate to the top substrate or from the top substrate to the bottom substrate. The substrate retention features of FIG. 33K may be applied to any sample analysis region disclosed herein.

In FIG. 33L, the sample analysis region 3370 comprises a quaternary zone 3372 (i.e., hydrophilic liquid well) that has substrate retention features 3373a-d and 3374a-b. The substrate retention features 3373a-d are quarter circle shaped and protrude into the quaternary zone 3372 and are located on the right end 3371. The substrate retention feature 3374a-b is located prior to the right end 3371 of the sample analysis region 3370 and are rectangular shaped. The substrate retention features 3374a-b may protrude from the bottom substrate to the top substrate or from the top substrate to the bottom substrate. The substrate retention features of FIG. 33L may be applied to any sample analysis region disclosed herein.

In FIG. 33M, the sample analysis region 3375 comprises a quaternary zone 3377 (i.e., hydrophilic liquid well) that has substrate retention features 3378a-d and 3379a-b. The substrate retention features 3378a-d are quarter circle shaped and protrude into the quaternary zone 3377 and are located on the right end 3376. The substrate retention feature 3379a-b is located prior to the right end 3376 of the sample analysis region 3375 and are circular shaped. The substrate retention features 3379a-b may protrude from the bottom substrate to the top substrate or from the top substrate to the bottom substrate. The substrate retention features of FIG. 33M may be applied to any sample analysis region disclosed herein.

In FIG. 33N, the sample analysis region 3380 comprises a quaternary zone 3382 (i.e., hydrophilic liquid well) that has substrate retention features 3383a-d and 3384. The substrate retention features 3383a-d are quarter circle shaped and protrude into the quaternary zone 3382 and are located on the right end 3381. The substrate retention feature 3384 is located on the right end 3381 of the sample analysis region 3380 and is horseshoe shaped. The substrate retention feature 3384 may protrude from the bottom substrate to the top substrate or from the top substrate to the bottom substrate. The substrate retention features of FIG. 33K may be applied to any sample analysis region disclosed herein.

FIG. 33O discloses an illustration of an exemplary barrier feature. The sample analysis region 3385 comprises a quinary zone 3386 (e.g., a hydrophobic liquid well) on the left end 3387. The sample analysis zone also comprises a barrier feature 3388. The barrier feature 3388 is half circle shaped. The barrier feature 3388 may protrude from the bottom substrate to the top substrate or from the top substrate to the bottom substrate. The barrier feature of FIG. 33O may be applied to any sample analysis region disclosed herein. A barrier feature may be configured to assist with the sealing of wells using the hydrophobic liquids disclosed herein.

FIG. 11 discloses an illustration of an isometric view of an embodiment of the device. The dotted lines represent interior portions of the device whereas solid lines represent exterior portions of the device. In this embodiment, the device 1100 contains a non-grid pattern (i.e., a honeycomb pattern) of primary zones 1103 and secondary zones 1106 in addition to a fixed primary zone 1108. In some instances, but not all instances, the fixed primary zone 1108 is also a sample mixing zone. The device comprises a reagent/sample injection port 1109. The device comprises a sample processing region 1105. The sample processing region 1105 is unbounded. By unbounded it is meant that there is no physical barrier separating the primary zones 1103 and the secondary zones 1106. The sample processing region comprises a primary zone 1103 that has an opening 1104 in the primary zone. The primary zone is adjacent to a secondary zone such as 1106. The sample processing region 1105 may be connected to a tertiary zone (i.e., the sample detection zone) 1102. The device may further comprise a quaternary zone 1107 (i.e., a hydrophilic liquid well) that is connected to the tertiary zone. The device may further comprise a quinary zone 1101 (i.e., an hydrophobic liquid well) that is connected to the tertiary zone.

FIG. 12 depicts an illustration of the top view of the embodiment of the device depicted in FIG. 11. 1201 refers to the reagent/sample injection port. 1202 refers to the fixed primary zone. 1206 refers to a primary zone. 1207 refers to a secondary zone. 1205 refers to a sample analysis region. 1203 refers to the hydrophilic liquid well. 1204 refers to the hydrophobic liquid well.

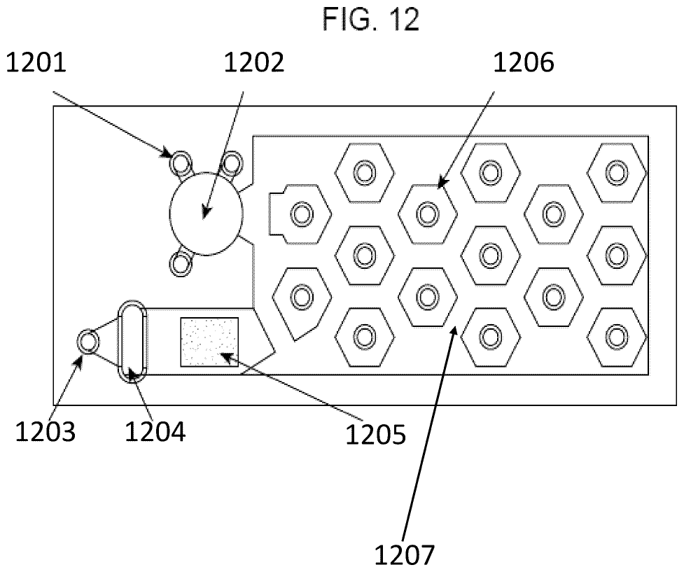

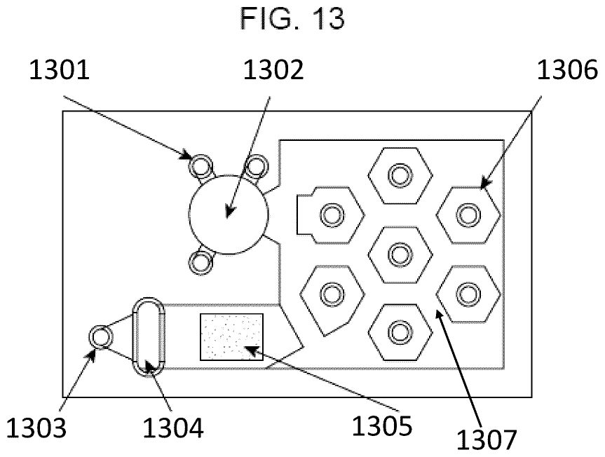

FIG. 13 depicts an embodiment of the device of FIG. 12 with reduced size and primary zones. 1301 refers to the reagent/sample injection port. 1302 refers to the fixed primary zone. 1306 refers to a primary zone. 1307 refers to a secondary zone. 1305 refers to a sample analysis region. 1303 refers to the hydrophilic liquid well. 1304 refers to the hydrophobic liquid well.

FIG. 14 depicts an illustration of the bottom view of the top substrate of the device according to FIG. 8. 1401 refers to the sample processing region. 1402 refers to the fixed primary zone. 1403 refers to a primary zone. 1404 refers to the opening of a primary zone. 1407 refers to a secondary zone. 1406 refers to an opening in a secondary zone.

FIG. 15 depicts an illustration of the bottom view of the top substrate of the device according to FIG. 7. 1501 refers to the sample processing region. 1502 refers to the fixed primary zone. 1503 refers to a primary zone. 1504 refers to the opening of a primary zone. 1507 refers to a secondary zone. 1506 refers to an opening in a secondary zone. 1408 refers to the sample detection region

FIG. 32B depicts an illustration of the bottom view of the top substrate of the device according to FIG. 32A. 3210 refers to the sample processing region. 3211 refers to the fixed primary zone. 3212 refers to a primary zone. 3213 refers to the opening of a primary zone. 3214 refers to a secondary zone. 3215 refers to an opening in a secondary zone.

### I. Arrangement of the Sample Processing Region

The sample processing regions of the present disclosure have a range of different arrangements such as those described above. The sample processing regions disclosed herein also have a range in the number of primary zones present. For example, the device may comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24 or more primary zones.

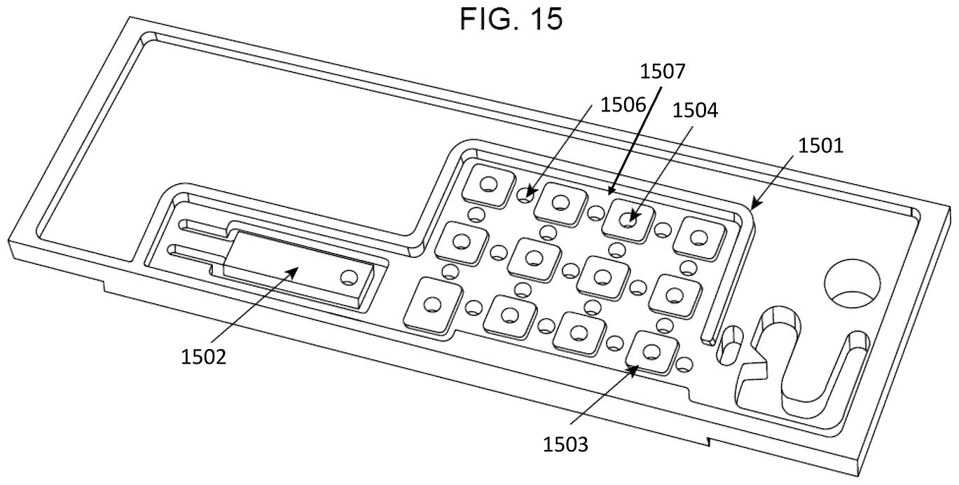

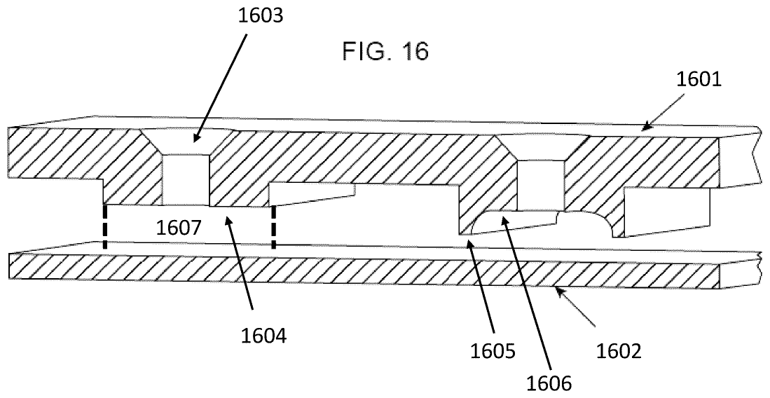

The primary zones of the present disclosure have pads that are formed from the top substrate. The pads found in the primary zones may have a wide range of shapes including, without limitation, a rectangle, a circle, a triangle, a pentagon, a hexagon, a heptagon, an octagon, a decagon, a dodecagon, an amoeboid, a non-regular shape, or a circular shape with points. In some embodiments, the pad is a rectangle or square such as those depicted in FIG. 1-7. In some embodiments, the pad is a circle such as those depicted in FIGS. 8 and 14. In some embodiments, the pad is a hexagon such as those depicted in FIG. 11-13. In some embodiments, the pad is an amoeboid such as those depicted in FIG. 27A. In some embodiments, the pad is a non-regular shape. In some embodiments, the pad is a circular shape with points such as those depicted in FIG. 27A. The pads in the primary zone have edges. In some embodiments, the edge is sharp. In some embodiments, the edge is curved such that the edge has a rounded shape. The pads of the primary zone may have a particular profile. In some embodiments, the pad is flat. In some embodiments, the pad has protrusions on the edges that are closer to the bottom substrate than the center of the pad such as depicted FIG. 16.

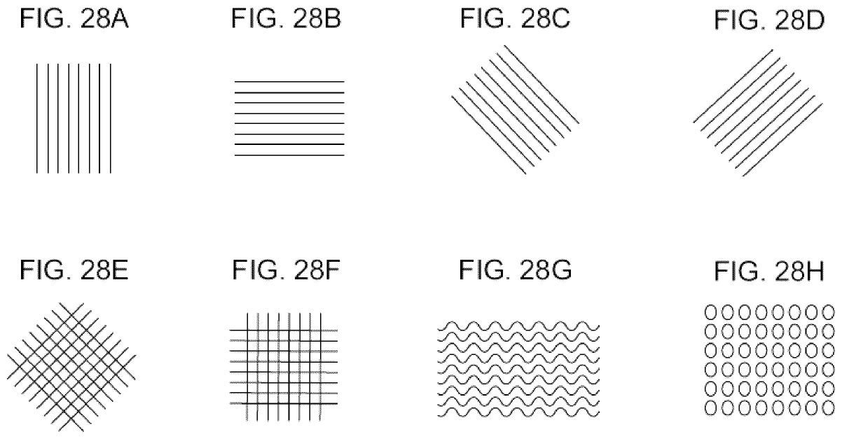

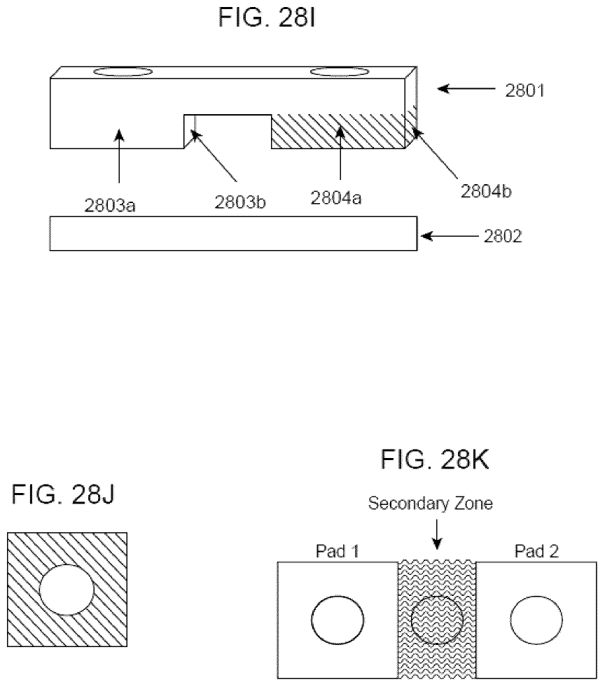

One or more surfaces of the pads may be designed in such a way that they retain fluid. The term “surface of the pad” refers to any surface of the pad that is facing the interior of the device such as the side of the pad that is facing another pad, the side of the pad that is facing the edge of the interior of the device, or the side of the pad that is facing the bottom substrate in the interior of the device. Alternative terms for the surfaces of the pad are “leading face” and “side surfaces”, wherein “leading face” refers to the surface of the pad that is facing the bottom substrate and “side surfaces” refer to the surfaces other than the “leading face”; when using this terminology and the device is planar, the “leading face” may be substantially parallel to the first plane and the “side surfaces” may be substantially perpendicular to the first plane. For instance, the one or more surfaces of the pads may contain serrations or grooves that occupy all or a portion of a given pad. The one or more surfaces of the pads may be two or more, three or more, four or more, five or more, six or more, seven or more, eight or more, nine or more, ten or more, or all of the surfaces of pad. The serrations or grooves may be in a range of different patterns including, without limitation, waves, straight lines that are perpendicular or parallel to the edge of the pad, diagonal to the edge of the pad, cross or hatch pattern, any combination thereof, etc. In some embodiments, the serrations or grooves are in a wave pattern such as depicted in FIG. 28G. In some embodiments, the serrations or grooves are in straight lines that are perpendicular or parallel to the edge of the pad such as depicted in FIG. 28A-B. In some embodiments, the serrations or grooves are in straight lines that are diagonal to the edge of the pad such as depicted in FIG. 28C-D. In some embodiments, the serrations or grooves are in a cross or hatch pattern such that two or more serrations or grooves cross one another such as depicted in FIG. 28E-F. In some embodiments, the serrations or grooves are uniformly spaced. In some embodiments, the serrations or grooves are not uniformly spaced. In some embodiments, all of the pads in the sample processing region contain serration or grooves. In some embodiments, only a portion of the pads in the sample processing region contain grooves or serrations.

FIG. 28I depicts an illustration of a pad in a primary zone with serrations or grooves and a pad in a primary zone without serrations or grooves. The top substrate 2801 and the bottom substrate 2802 define the interior of the device. The pad on the left has surfaces 2803a and 2803b that do not have serrations or grooves. The pad has three additional surfaces not depicted, one surface opposite surface 2803a, one opposite surface 2803b, and one surface facing the bottom substrate 2802. The pad on the right has serrations or grooves that are diagonal to the edge of the pad on surface 2804a and 2804b. The pad may have additional surfaces with serrations or grooves such as the surface of the pad facing the bottom surface 2802 such as depicted in FIG. 28J, the surface of the pad opposite 2804a, and the surface of the pad opposite 2804b.

In some embodiments, the one or more surfaces of the pad contain dimples or divots such as depicted in FIG. 28H. In some embodiments, the dimples or divots are concave. In some embodiments, the dimples or divots are convex (e.g., protrusions). In some embodiments, a portion of the dimples or divots are convex and portion of the dimples or divots are concave. In some embodiments, the dimples or divots are circular in shape. In some embodiments, the dimples or divots are triangular in shape. In some embodiments, the dimples or divots are rectangular in shape. In some embodiments, the dimples or divots are pentagonal in shape. In some embodiments, the dimples or divots are cylindrical in shape. In some embodiments, the dimples or divots are hexagonal in shape. In some embodiments, the dimples or divots are heptagonal in shape. In some embodiments, the dimples or divots are octagonal in shape. In some embodiments, the dimples or divots are decagonal in shape. In some embodiments, the dimples or divots are dodecagonal in shape. In some embodiments, the dimples or divots are amoeboid in shape. In some embodiments, the dimples or divots are non-regular in shape. In some embodiments, the dimples or divots are uniformly spaced. In some embodiments, the dimples or divots are not uniformly spaced. In some embodiments, the serrations or grooves are not uniformly spaced. In some embodiments, all of the pads in the sample processing region contain dimples or divots. In some embodiments, only a portion of the pads in the sample processing region contain dimples or divots. In some embodiments, a portion of the pads in the sample processing region contain dimple or divots and a portion of the pads contain serrations or grooves. In some embodiments, all of or a portion of the pads in the sample processing region contain serrations or grooves and dimples or divots.

FIG. 16 depicts an illustration of a cross-section of the primary zones and pads of an embodiment of the device. A primary zone 1607 (between the dotted lines) is formed by the top substrate 1601 and the bottom substrate 1602. The primary zone contains a pad 1604 or 1605. In some embodiments, the pad is flat (1604). In some embodiments, the pad has a protrusion 1605 around the entire edge such that the protrusion 1605 is closer to the bottom substrate 1602 than the center of the pad 1606.

The pads of the present disclosure have a range of thickness. For instance, the pad may about 0.3 mm, 0.4 mm, 0.5 mm, 0.6 mm, 0.7 mm, 0.8 mm, 0.9 mm, or about 1.0 mm. In some embodiments, the pad has a thickness between 0.3 mm and 0.8 mm.

The pads in the primary zone and the bottom substrate in the primary zone may have a particular patterning. The patterning may be designed to retain fluid in the primary zone. In some embodiments, the pad and the bottom substrate have hydrophilic patterning. In some embodiments, the pad and the bottom substrate in the primary zone do not have any patterning.

The sample processing regions disclosed herein also have a range in the number of secondary zones present. The device may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, or 37 or more secondary zones.

The secondary zones of the present disclosure are formed by the top and bottom substrates of the device. The secondary zones are present between one or more primary zones. In some embodiments, the secondary zone is between two primary zones. The secondary zones have a distance from the top substrate to the bottom substrate that is greater than the distance from the top substrate to the bottom substrate in the primary zones. The difference in the height between the primary zone and secondary zone creates surface tension that is able to hold a fluid in the primary zone without spilling into the secondary zone. The difference in the height between the primary zone and the secondary zone is depicted in FIG. 16 and FIG. 26. In some embodiments, the top substrate and the bottom substrate of the secondary zone have hydrophobic patterning. In some embodiments, the top substrate and the bottom substrate of the secondary zones have no patterning. In some embodiments, the top substrate and the bottom substrate of the secondary zones have uniform hydrophobicity.

The secondary zones may be designed in such a way that they retain fluid. For instance, the roof of the secondary zone (i.e., the bottom side of the top substrate facing the interior of the device) may contain serrations or grooves that occupy all or a portion of a given roof of a secondary zone. In some embodiments, the serrations or grooves are in a wave pattern such as depicted in FIG. 28G and FIG. 28K. In some embodiments, the serrations or grooves are in straight lines that are perpendicular or parallel to the edge of the pad adjacent to the secondary zone such as depicted in FIG. 28A-B. In some embodiments, the serrations or grooves are in straight lines that are diagonal to the edge of the pad adjacent to the secondary zone such as depicted in FIG. 28C-D. In some embodiments, the serrations or grooves are in a cross or hatch pattern such that two or more serrations or grooves cross one another such as depicted in FIG. 28E-F. In some embodiments, the serrations or grooves are uniformly spaced. In some embodiments, the serrations or grooves are not uniformly spaced. In some embodiments, all of the roofs of the secondary zones in the sample processing region contain serration or grooves. In some embodiments, only a portion of the roofs of the secondary zones in the sample processing region contain grooves or serrations.

In some embodiments, the roof of the secondary zone contains dimples or divots such as depicted in FIG. 28H. In some embodiments, the dimples or divots are concave. In some embodiments, the dimples or divots are convex (e.g., protrusions). In some embodiments, a portion of the dimples or divots are convex and portion of the dimples or divots are concave. In some embodiments, the dimples or divots are circular in shape. In some embodiments, the dimples or divots are triangular in shape. In some embodiments, the dimples or divots are rectangular in shape. In some embodiments, the dimples or divots are pentagonal in shape. In some embodiments, the dimples or divots are cylindrical in shape. In some embodiments, the dimples or divots are hexagonal in shape. In some embodiments, the dimples or divots are heptagonal in shape. In some embodiments, the dimples or divots are octagonal in shape. In some embodiments, the dimples or divots are decagonal in shape. In some embodiments, the dimples or divots are dodecagonal in shape. In some embodiments, the dimples or divots are amoeboid in shape. In some embodiments, the dimples or divots are non-regular in shape. In some embodiments, the dimples or divots are uniformly spaced. In some embodiments, the dimples or divots are not uniformly spaced. In some embodiments, all of the roofs of the secondary zones in the sample processing region contain dimples or divots. In some embodiments, only a portion of the roofs of the secondary zones in the sample processing region contain dimples or divots. In some embodiments, a portion of the roofs of the secondary zones in the sample processing region contain dimple or divots and a portion of the roofs of the secondary zones contain serrations or grooves. In some embodiments, all of or a portion of the roofs in the secondary zones in the sample processing region contain serrations or grooves and dimples or divots.

In some embodiments, the top substrate has uniform hydrophobicity such that the entire surface of the top substrate is hydrophobic, hydrophilic, or is not hydrophobic or hydrophilic. In some embodiments, the bottom substrate has uniform hydrophobicity such that the entire surface of the bottom substrate is hydrophobic, hydrophilic, or is not hydrophobic or hydrophilic.

The primary zones of the present disclosure have an opening in the top substrate such that air or fluid may be added to the primary zone through the opening, either passively or actively. The opening may also produce capillary forces when a fluid is present in the primary zone such that the capillary forces hold the fluid in the primary zone without it spilling into the secondary zone. The opening in the primary zones has a range of different diameters. The diameter of opening may be about 0.2, 0.3, 0.4, 0.5, 0.6, 0.7, 0.8, 0.9, 1.0, 1.1. 1.2, 1.3, 1.4, or about 1.5 mm.

In some embodiments, the secondary zones have an opening in the top substrate. In some embodiments, the secondary zones do not have an opening in the top substrate. When the secondary zone has an opening in the top substrate, the opening is such that air or fluid may be added to the secondary zone, either passively or actively. The opening in the secondary zones has a range of different diameters. The diameter of opening may be about 0.8, 0.9, 1.0, 1.1. 1.2, 1.3, 1.4, or about 1.5 mm.

In some embodiments, the sample processing region contains a sample mixing zone. In some embodiments, the sample mixing zone and the fixed primary zone are the same. In some embodiments, the sample processing region comprises two or more sample mixing zones. In some embodiments, the sample processing region comprises a fixed primary zone that is a sample mixing zone and one or more additional sample mixing zones. In some embodiments, the sample processing region comprises a fixed primary zone that is not a sample mixing zone and a sample mixing zone. FIGS. 7, 8, and 10 disclose exemplary sample mixing zones.

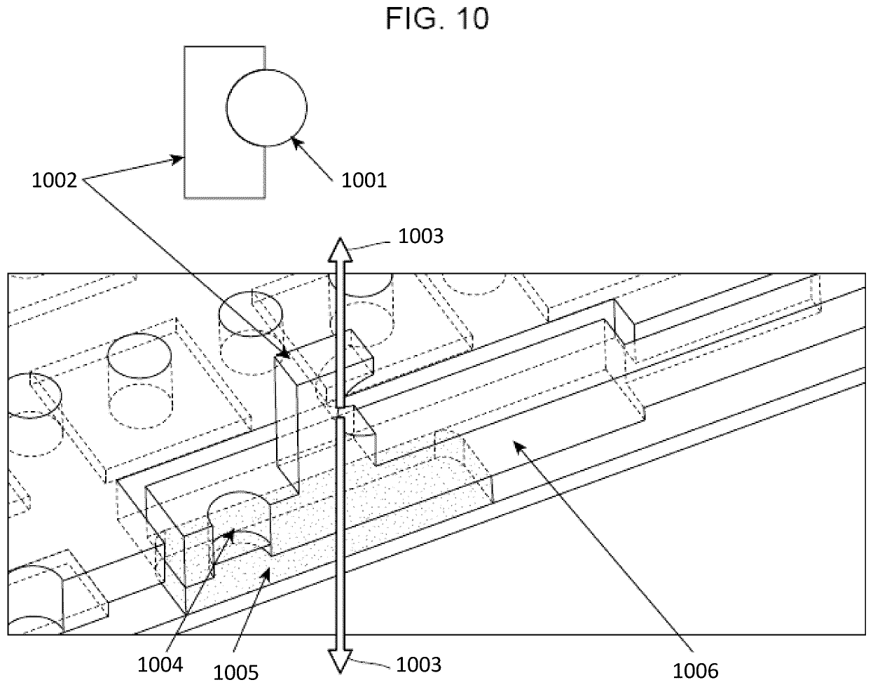

FIG. 10 discloses an illustration of exemplary sample mixing zone. In this embodiment, the sample mixing zone comprises a fixed primary zone 1006 containing a sample 1005. The sample is added through the opening 1004 in the fixed primary zone. The fixed primary zone 1006 contains a hooked portion 1002. The hooked portion is capable of joining with a vibration source 1001. The vibration source provides vertical motion 1003 that compresses and decompresses the sample 1005. The compression and decompression of the sample 1005 results in the mixing of the sample. The fixed primary zone 1006 is a cantilever in that only the portion opposite the opening 1004 is attached to the top substrate. All other regions around the fixed primary zone are detached from the top substrate.

FIG. 32A discloses an alternative embodiment to FIG. 10 where the sample mixing zone does not contain a hooked portion. In this embodiment, the sample mixing zone comprises a fixed primary zone 3200. The sample is added through the opening 3201 in the fixed primary zone In this embodiment, the fixed primary zone contains two openings. In some embodiments, the fixed primary zone one or more openings. In some embodiments, the fixed primary zone contains three or more openings. The fixed primary zone 3200 does not contain a hooked portion. A vibration source comes in contact with the end 3202 of the fixed primary zone. The vibration source provides vertical motion that compresses and decompresses the sample. The compression and decompression of the sample results in the mixing of the sample. The fixed primary zone 3200 is a cantilever in that only the portion opposite the end 3202 is attached to the top substrate. All other regions around the fixed primary zone are detached from the top substrate.

### II. Arrangement of the Sample Analysis Region

The optional sample analysis region of the devices of the present disclosure contains a number of different regions including, without limitation, a transition zone, a tertiary zone (e.g., a sample detection zone), a quaternary zone (e.g., hydrophilic liquid well or reservoir), a quinary zone (e.g., a hydrophobic liquid well or reservoir), or any combination thereof.

In some embodiments, the sample analysis region comprises a transition zone. The transition zone facilities the transfer of the microparticles comprising the sample or analytes contained in the sample from the sample processing region to the sample analysis region. In some embodiments, the transition zone is air-filled. The transition zone may be any length that sufficiently separates the sample processing region from the sample analysis region such that little or no fluid is transferred from the sample processing region to the sample analysis region.

In some embodiments, the sample analysis region comprises a tertiary zone. In some embodiments, the tertiary zone is a sample detection zone. The types of sample detection zones include, without limitation, wells or microwells, chambers, nanopores, etc. In some embodiments, the sample analysis region comprises hydrophobic liquid and hydrophilic liquid wells.

In some embodiments, the sample analysis region comprises a sample detection zone comprising wells. The wells may have sub-femtoliter volume, femtoliter volume, sub-nanoliter volume, nanoliter volume, sub-microliter volume, or microliter volume. For example, wells may be femoliter wells, nanoliter wells, or microliter wells. In certain embodiments, the wells in an array may all have substantially the same volume. The array of wells may have a volume up to 100 microliter, e.g., about 0.1 femtoliter, 1 femtoliter, 10 femtoliter, 25 femtoliter, 50 femtoliter, 100 femtoliter, 0.1 pL, 1 μL, 10 pL, 25 μL, 50 pL, 100 μL, 0.1 nL, 1 nL, 10 nL, 25 nL, 50 nL, 100 nL, 0.1 microliter, 1 microliter, 10 microliter, 25 microliter, 50 microliter, or 100 microliter.

In some examples, the wells are an array of wells that include a plurality of individual wells. The array of wells may include a plurality of wells that may range from 109 to 10 in number per 1 mm². In certain cases, an array of about 100,000 to 500,000 wells (e.g., femtoliter wells) covering an area approximately 12 mm² may be fabricated. Each well may measure about 4.2 μm wide×3.2 μm deep (volume approximately 50 femtoliters) and may be capable of holding a single microparticle (about 3 μm diameter). At this density, the femtoliter wells are spaced at a distance of approx. 7.4 μm from each other. In some examples, the well array may be fabricated to have individual wells with a diameter of 10 nm to 10,000 nm.

The placement of single microparticles bound to the analyte molecules in the wells allows for either a digital readout or analog readout. For example, for a low number of positive wells (<˜ 70% positive) Poisson statistics can be used to quantitate the analyte concentration in a digital format; for high numbers of positive wells (>˜70%) the relative intensities of signal-bearing wells are compared to the signal intensity generated from a single microparticle bound to the analyte molecule, respectively, and used to generate an analog signal. A digital signal may be used for lower analyte concentrations, whereas an analog signal may be used for higher analyte concentrations. A combination of digital and analog quantitation may be used, which may expand the linear dynamic range. In some embodiments, the signal intensity of a well increases overtime indicating the presence of more than analyte in the well. In some embodiments, the signal intensity in a well is stagnant and of higher intensity of wells containing a single analyte indicating the presence of more than analyte in the well As used herein, a “positive well” refers to a well that has a signal related to presence of a microparticle bound to the analyte molecule, which signal is above a threshold value. As used herein, a “negative well” refers to a well that may not have a signal related to presence of a microparticle bound to the analyte molecule. In certain embodiments, the signal from a negative well may be at a background level, i.e., below a threshold value.

The wells may be any of a variety of shapes, such as, cylindrical with a flat bottom surface, cylindrical with a rounded bottom surface, cubical, cuboidal, frustoconical, inverted frustoconical, pentagonal, triangular, pyramidal, or conical. In certain cases, the wells may include a sidewall that may be oriented to facilitate the receiving and retaining of a microparticle present liquid droplets that have been moved over the well array. In certain cases, the wells may include a sidewall that may be oriented to facilitate the receiving and retaining of a microparticle that is not present in a liquid droplet that have been moved over the well array. In some examples, the wells may include a first sidewall and a second sidewall, where the first sidewall may be opposite the second side wall. In some examples, the first sidewall is oriented at an obtuse angle with reference to the bottom of the wells and the second sidewall is oriented at an acute angle with reference to the bottom of the wells. In some embodiments, the movement of the droplets is in a direction parallel to the bottom of the wells and from the first sidewall to the second sidewall. In some embodiments, the movement of the microparticle is in a direction parallel to the bottom of the wells and from the first sidewall to the second sidewall.

In some embodiments, the sample analysis region comprises a sample detection zone comprising a chamber. The chamber may be a range of different sizes and shapes such that the chamber is suitable for the detection of the analyte. The chamber may accommodate a reaction mixture having a volume of from 5 microliters to 1 milliliter. For example, the reaction chamber may be sized to contain a reaction mixture having a volume of from 5 microliters to 500 microliters. According to certain embodiments, the reaction chamber is sized to contain a reaction mixture having a volume of from 5 microliters to 100 microliters. In some embodiments, the fluid capacity of the chamber is 1 mL or less, 750 μL or less, 500 μL or less, 400 μL or less, 300 μL or less, 250 μL or less, 200 μL or less, 150 μL or less, 100 μL or less, 50 μL or less, or 25 μL or less.

The chamber may be a range of different shapes in order to retain the analyte within or without a reaction mixture. In some embodiments, the shape of the chamber is a cube. In some embodiments, the shape of the chamber is a cylinder. In some embodiments, the shape of the chamber is hexagonal. In some embodiments, the shape of the chamber is a sphere. In some embodiments, the shape of the chamber is octagonal. In some embodiments, the shape of the chamber is conical. In some embodiments, the shape of the chamber is cuboidal.

The bottom, sides, or top of the chamber may be optical transparent such that the analyte may be detected through optical means. In some embodiments, all sides of the chamber are optically transparent such that the analyte may be detected through optical means. In some embodiments, only the top and the bottom of the chamber are optically transparent such that the analyte may be detected through optical means. In some embodiments, the top of the chamber is reflective and the bottom of the chamber is transparent. In some embodiments, the bottom of the chamber is transparent and the bottom of the chamber is reflective.

In some embodiments, the chamber is a reaction vessel. The term “reaction vessel” as used herein generally refers to a container within which an amplification reaction, an immunoassay, clinical chemistry, or complete blood component analysis is performed. The reaction vessel may be obtained from commercial sources, e.g., as off-the-shelf components such as a microamp tube or microamp tubes joined together in a 96 well format, or may be custom manufactured. Reaction vessels useful in nucleic acid amplification reactions will generally be capable of rapidly transferring heat across the vessel, e.g., through the use of highly conductive materials (e.g., thermally conductive plastics) or physical modifications of the vessel (e.g., thin walls). Common reaction vessels include but are not limited to e.g., tubes, vials, multi-well plates, and the like. Reaction vessels may be constructed of a variety of materials including but not limited to e.g., polymeric materials.

The top opening of the reaction vessel may be any convenient shape. In certain aspects, the top opening of the reaction chamber of the reaction vessel is circular. The shape of the reaction chamber may vary. According to certain embodiments, the reaction chamber has a conical shape. The bottom of the reaction vessel may be flat. In other aspects, the reaction vessel has a round bottom.

In certain aspects, the wall of the reaction chamber is straight, whereby “straight” is meant the wall does not include a “step” (or “ridge”). In other aspects, the wall of the reaction chamber includes one or more (e.g., 2 or more, 3 or more, 4 or more, etc.) steps. The one or more steps may be complementary to the shape of the a cap for the reaction vessel. For example, according to certain embodiments, the reaction vessel includes a step that forms an upper region and a lower region of the reaction vessel, where the shape of the upper region is complementary to the shape of the reaction vessel cap.

The volume of the reaction vessel may vary. In certain aspects, the reaction chamber is sized to contain a reaction mixture having a volume of from 1 microliter to 500 microliters. For example, the reaction chamber may be sized to contain a reaction mixture having a volume of from 1 microliters to 10 microliters, 10 microliters to 50 microliters, 50 microliters to 100 microliters, 100 microliters to 200 microliters, 200 microliters to 300 microliters, 300 microliters to 400 microliters, 400 microliters to 500 microliters, etc. According to certain embodiments, the reaction chamber is sized to contain a reaction mixture having a volume of from 1 microliters to 200 microliters.

In certain aspects, the fluid capacity of the reaction vessel is 500 mL or less, 450 μL or less, 400 μL or less, 350 μL or less, 300 μL or less, 250 μL or less, 200 μL or less, 150 μL or less, 100 μL or less, 50 μL or less, 25 μL or less, 20 μL or less, 15 μL or less, 10 μL or less, or 5 μL or less.

The external surface of the reaction vessel may include a variety of shapes and features. In certain aspects, the bottom surface of the reaction vessel is round. In other aspects, the bottom surface of the reaction vessel is flat.

The bottom, sides, or top of the reaction vessel may be optical transparent such that the analyte may be detected through optical means. In some embodiments, all sides of the reaction vessel are optically transparent such that the analyte may be detected through optical means. In some embodiments, only the top and the bottom of the reaction vessel are optically transparent such that the analyte may be detected through optical means. In some embodiments, only the top of the reaction vessel is optically transparent such that the analyte may be detected through optical means. In some embodiments, only the bottom of the reaction vessel is optically transparent such that the analyte may be detected through optical means.

In certain instances, the reaction vessels may be those described in U.S. Pat. No. 10,648,018 which is specifically incorporated by reference herein.

In some embodiments, the sample analysis region contains nanopores. When the sample detection zone comprises nanopores, the analyte or analyte molecule (e.g., a nucleic acid, a non-nucleic acid containing a nucleic acid tag, or a nucleic acid produced from the analyte) is detected by translocating the nucleic acid through or across a nanopore. In some embodiments, detecting the analyte may be carried out by translocating the nucleic acid through or across at least one or more nanopores. In some embodiments, at least two or more nanopores are presented side by side or in series. In some embodiments, the nanopore is dimensioned for translocation of not more than one nucleic acid at a time. Thus, the dimensions of the nanopore in some embodiments will typically depend on the dimensions of the nucleic acid to be examined. A nucleic acid with a double-stranded region can require a nanopore dimension greater than those sufficient for translocation of a nucleic acid which is entirely single-stranded. In addition, a microparticle-associated nucleic acid such as a microparticle tag can require larger nanopores than oligomer tags. Typically, a nanopore of about 1 nm diameter can permit passage of a single-stranded polymer, while nanopore dimensions of 2 nm diameter or larger will permit passage of a double-stranded nucleic acid. In some embodiments, the nanopore is selective for a single-stranded tag (e.g., from about 1 nm to less than 2 nm diameter) while in other embodiments, the nanopore is of a sufficient diameter to permit passage of double-stranded polynucleotides (e.g., 2 nm or larger). The chosen nanopore size provides an optimal signal-to-noise ratio for the analyte of interest.

In some embodiments, the nanopore may be between about 0.1 nm and about 1000 nm in diameter, between about 50 nm and about 1000 nm, between about 100 nm and 1000 nm, between about 0.1 nm and about 700 nm, between about 50 nm and about 700 nm, between about 100 nm and 700 nm, between about 0.1 nm and about 500 nm, between about 50 nm and about 500 nm, or between about 100 nm and 500 nm. For example, the nanopore may be about 0.1 nm, about 0.2 nm, about 0.3 nm, about 0.4 nm, about 0.5 nm, about 0.6 nm, about 0.7 nm, about 0.8 nm, about 0.9 nm, about 1.0 nm, about 1.5 nm, about 2.0 nm, about 2.5 nm, about 3.0 nm, about 3.5 nm, about 4.0 nm, about 4.5 nm, about 5.0 nm, about 7.5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250 nm, about 300 nm, about 3500 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm in diameter.

Various types of nanopores may be used for analyzing the nucleic acid present in a sample. These include, among others, biological nanopores that employ a biological nanopore embedded in a membrane. Another type of nanopore layer is a solid state nanopore in which the nanopore is made whole or in part from a fabricated or sculpted solid-state component, such as silicon. In some embodiments, the nanopore is a solid-state nanopore produced using controlled dielectric breakdown. In some embodiments, the nanopore is a solid-state nanopore produced by a method other than controlled dielectric breakdown.

In certain embodiments, the length of a nanopore may be up to about 200 nm, e.g., from about 0.1 nm to about 30 nm, from about 10 to about 80 nm, from about 1 to about 50 nm, from about 0.1 nm to about 0.5 nm, from about 0.3 nm to about 1 nm, from about 1 nm to about 2 nm, from about 0.3 nm to about 10 nm, or from about 10 to about 30 nm. The number of nanopores in a nanopore layer may be about 1, 2, 3, 4, 5, 10, 30, 100, 300, 1000, 3000, 10000, 30000, 100000, 300000 or more. The distance between nanopores in a layer between center to center may be about 100 nm to about 300 nm, about 300 nm to about 500 nm, about 500 nm to about 1000 nm, for example, 100 nm, 150 nm, 200 nm, or 300 nm.

In certain embodiments, multiple nanopore layers, each containing one or more nanopores, can be arranged in series with each other, for detecting and/or counting the tag (e.g., polymer, aptamer, microparticle). In this case, detecting and/or counting the nucleic acid may be carried out by translocating the nucleic acid through or across each nanopore layer. As such, counting the number of nucleic acids translocating through or across a nanopore in a layer/sheet/membrane refers to counting multiple nucleic acids translocating through or across one or more nanopores in one or more layer/sheet/membrane. In certain embodiments, when more than one nanopore layers are present (e.g., one, two, three, four, five, six, or other number of nanopore layers as technically feasible), optionally they are present in series wherein at least one nanopore in one layer is separate from or stacked onto (e.g., above or on top of) another nanopore in another layer, etc.). Where the nanopore layers are in series, at least two electrodes can be used to create an electric field to drive tags through the pores and, optionally, additional electrodes positioned between the nanopore layers can further provide driving current.

### Biological Nanopores

For detecting and, optionally, counting the nucleic acid, any biological nanopore with channel dimensions that permit translocation of the nucleic can be used. Two broad categories of biological nanopores are suitable for the methods disclosed herein. Non-voltage gated nanopores allow passage of molecules through the nanopore without requiring a change in the membrane potential to activate or open the channel. On the other hand, voltage gated nanopores require a particular range of membrane potential to activate nanopore opening. Most studies with biological nanopores have used a-hemolysin, a mushroom-shaped homo-oligomeric heptameric channel of about 10 nm in length found in Staphylococcus aureus. Each subunit contributes two beta strands to form a 14 strand anti-parallel beta barrel. The nanopore formed by the beta barrel structure has an entrance with a diameter of approximately 2.6 nm that contains a ring of lysine residues and opens into an internal cavity with a diameter of about 3.6 nm. The stem of the hemolysin nanopore, which penetrates the lipid bilayer, has an average inside diameter of about 2.0 nm with a 1.5 nm constriction between the vestibule and the stem. The dimensions of the stem are sufficient for passage of single-stranded nucleic acids but not double-stranded nucleic acids. Thus, a-hemolysin nanopores may be used as a nanopore selective for single-stranded polynucleotides and other polymers of similar dimensions.

In other embodiments, the biological nanopore is of a sufficient dimension for passage of polymers larger than a single-stranded nucleic acid. An exemplary nanopore is mitochondrial porin protein, a voltage-dependent anion channel (VDAC) localized in the mitochondrial outer membrane. Porin protein is available in purified form and, when reconstituted into artificial lipid bilayers, generates functional channels capable of permitting passage of double-stranded nucleic acids (Szabo et al., 1998, FASEB J. 12:495-502). Structural studies suggest that porin also has a beta-barrel type structure with 13 or 16 strands (Rauch et al., 1994, Biochem Biophys Res Comm 200:908-915). Porin displays a larger conductance compared conductance of nanopores formed by a-hemolysin, maltoporin (LamB), and gramicidin. The larger conductance properties of porin support studies showing that the porin channel is sufficiently dimensioned for passage of double-stranded nucleic acids. Nanopore diameter of the porin molecule is estimated at 4 nm. The diameter of an uncoiled double-stranded nucleic acid is estimated to be about 2 nm.

Another biological nanopore that may be suitable for scanning double stranded polynucleotides are channels found in B. subtilis (Szabo et al., 1997, J. Biol. Chem. 272:25275-25282). Plasma membrane vesicles made from B. subtilis and incorporated into artificial membranes allow passage of double-stranded DNA across the membrane. Conductance of the nanopore formed by B. subtilis membrane preparations is similar to those of mitochondrial porin. Although there is incomplete characterization (e.g., purified form) of these nanopore, it is not necessary to have purified forms for the purposes herein. Diluting plasma membrane preparations, either by solubilizing in appropriate detergents or incorporating into artificial lipid membranes of sufficient surface area, can isolate single nanopores in a detection apparatus. Limiting the duration of contact of the membrane preparations (or protein preparations) with the artificial membranes by appropriately timed washing provides another method for incorporating single nanopores into the artificial lipid bilayers. Conductance properties may be used to characterize the nanopores incorporated into the bilayer.

In certain cases, the nanopores may be hybrid nanopores, where a biological nanopore is introduced in a solid state nanopore, e.g., a nanopore fabricated in a non-biological material. For example, a-haemolysin nanopore may be inserted into a solid state nanopore. In certain cases, the nanopores may be a hybrid nanopore described in Hall et al., Nature Nanotechnology, 28 Nov. 2010, vol. 5, pg. 874-877.

### Solid State Nanopores

In other embodiments, analysis of the nucleic acid is carried out by translocating the tag through or across a nanopore fabricated from non-biological materials. Nanopores can be made from a variety of solid-state materials using a number of different techniques, including, among others, chemical deposition, electrochemical deposition, electroplating, electron beam sculpting, ion beam sculpting, nanolithography, chemical etching, laser ablation, focused ion beam, atomic layer deposition, and other methods well known in the art (see, e.g., Li et al., 2001, Nature 412:166-169; and WO 2004/085609).

In particular embodiments, the nanopores may be the nanopores described in WO13167952A1 or WO13167955A1. As described in WO13167952A1 or WO13167955A1, nanopores having an accurate and uniform nanopore size may be formed by precisely enlarging a nanopore formed in a membrane. The method may involve enlarging a nanopore by applying a high electric potential across the nanopore; measuring current flowing through the nanopore; determining size of the nanopore based in part on the measured current; and removing the electric potential applied to the nanopore when the size of the nanopore corresponds to a desired size. In certain cases, the applied electric potential may have a pulsed waveform oscillating between a high value and a low value, the current flowing through the nanopore may be measured while the electric potential is being applied to the nanopore at a low value.

Solid state materials include, by way of example and not limitation, any known semiconductor materials, insulating materials, and metals coated with insulating material. Thus, at least part of the nanopore(s) may comprise without limitation silicon, silica, silicene, silicon oxide, graphene, silicon nitride, germanium, gallium arsenide, or metals, metal oxides, and metal colloids coated with insulating material.

To make a nanopore of nanometer dimensions, various feedback procedures can be employed in the fabrication process. In embodiments where ions pass through a hole, detecting ion flow through the solid state material provides a way of measuring pore size generated during fabrication (see, e.g., U.S. Published Application No. 2005/0126905). In other embodiments, where the electrodes define the size of the pore, electron tunneling current between the electrodes gives information on the gap between the electrodes. Increases in tunneling current indicate a decrease in the gap space between the electrodes. Other feedback techniques will be apparent to the skilled artisan.

In some embodiments, the nanopore is fabricated using ion beam sculpting, as described in Li et al., 2003, Nature Materials 2:611-615. In some embodiments, the nanopore is fabricated using high current, as described in WO13167952A1 or WO13167955A1. In other embodiments, the nanopores may be made by a combination of electron beam lithography and high energy electron beam sculpting (see, e.g., Storm et al., 2003, Nature Materials 2:537-540). A similar approach for generating a suitable nanopore by ion beam sputtering technique is described in Heng et al., 2004, Biophy J 87:2905-2911. The nanopores are formed using lithography with a focused high energy electron beam on metal oxide semiconductor (CMOS) combined with general techniques for producing ultrathin films. In other embodiments, the nanopore is constructed as provided in U.S. Pat. Nos. 6,627,067; 6,464,842; 6,783,643; and U.S. Publication No. 2005/0006224 by sculpting of silicon nitride.

In some embodiments, the nanopores can be constructed as a gold or silver nanopore. These nanopores are formed using a template of porous material, such as polycarbonate filters prepared using a track etch method, and depositing gold or other suitable metal on the surface of the porous material. Track etched polycarbonate membranes are typically formed by exposing a solid membrane material to high energy nuclear particles, which creates tracks in the membrane material. Chemical etching is then employed to convert the etched tracks to nanopores. The formed nanopores have a diameter of about 10 nm and larger. Adjusting the intensity of the nuclear particles controls the density of nanopores formed in the membrane. Nanopores are formed on the etched membrane by depositing a metal, typically gold or silver, into the track etched nanopores via an electroless plating method (Menon et al., 1995, Anal Chem 67:1920-1928). This metal deposition method uses a catalyst deposited on the surface of the nanopore material, which is then immersed into a solution containing Au(I) and a reducing agent. The reduction of Au(I) to metallic Au occurs on surfaces containing the catalyst. Amount of gold deposited is dependent on the incubation time such that increasing the incubation time decreases the inside diameter of the pores in the filter material. Thus, the nanopore size may be controlled by adjusting the amount of metal deposited on the pore. The resulting nanopore dimension is measured using various techniques, for instance, gas transport properties using simple diffusion or by measuring ion flow through the pores using patch clamp type systems. The support material is either left intact, or removed to leave gold nanopores. Electroless plating technique is capable of forming nanopore sizes from less than about 1 nm to about 5 nm in diameter, or larger as required. Gold nanopores having nanopore diameter of about 0.6 nm appears to distinguish between Ru(bpy)2+2 and methyl viologen, demonstrating selectivity of the gold nanopores (Jirage et al., 1997, Science 278:655-658). Modification of a gold nanopore surface is readily accomplished by attaching thiol containing compounds to the gold surface or by derivatizing the gold surface with other functional groups. This features permits attachment of nanopore modifying compounds as well as sensing labels, as discussed herein. Devices, such as the cis/trans apparatuses used for biological nanopores described herein, can be used with the gold nanopores to analyze single coded molecules.

Where the mode of detecting the analyte (e.g., a nucleic acid, a non-nucleic acid containing a nucleic acid tag, or a nucleic acid produced from the analyte) involves current flow through the analyte (e.g., electron tunneling current), the solid state membrane may be metalized by various techniques. The conductive layer may be deposited on both sides of the membrane to generate electrodes suitable for interrogating the tag along the length of the chain, for example, longitudinal electron tunneling current. In other embodiments, the conductive layer may be deposited on one surface of the membrane to form electrodes suitable for interrogating the analyte across the nanopore, for example, transverse tunneling current. Various methods for depositing conductive materials are known, including, sputter deposition (i.e., physical vapor deposition), non-electrolytic deposition (e.g., colloidal suspensions), and electrolytic deposition. Other metal deposition techniques are filament evaporation, metal layer evaporation, electron-beam evaporation, flash evaporation, and induction evaporation, and will be apparent to the skilled artisan.

In some embodiments, the detection electrodes are formed by sputter deposition, where an ion beam bombards a block of metal and vaporizes metal atoms, which are then deposited on a wafer material in the form of a thin film. Depending on the lithography method used, the metal films are then etched by means of reactive ion etching or polished using chemical-mechanical polishing. Metal films may be deposited on preformed nanopores or deposited prior to fabrication of the pore.

In some embodiments, the detection electrodes are fabricated by electrodeposition (see, e.g., Xiang et al., 2005, Angew. Chem. Int. Ed. 44:1265-1268; Li et al., Applied Physics Lett. 77 (24): 3995-3997; and U.S. Publication Application No. 2003/0141189). This fabrication process is suitable for generating a nanopore and corresponding detection electrodes positioned on one face of the solid state film, such as for detecting transverse electron tunneling. Initially, a conventional lithographic process is used to form a pair of facing electrodes on a silicon dioxide layer, which is supported on a silicon wafer. An electrolyte solution covers the electrodes, and metal ions are deposited on one of the electrodes by passing current through the electrode pair. Deposition of metal on the electrodes over time decreases the gap distance between the electrodes, creating not only detection electrodes but a nanometer dimensioned gap for translocation of coded molecules. The gap distance between the electrodes may be controlled by a number of feedback processes.

Where the detection is based on imaging of charge induced field effects, a semiconductor can be fabricated as described in U.S. Pat. No. 6,413,792 and U.S. published application No. 2003/0211502. The methods of fabricating these nanopore devices can use techniques similar to those employed to fabricate other solid state nanopores.

Detection of the analyte, such as a polynucleotide, is carried out as further described below. For analysis of the analyte, the nanopore may be configured in various formats. In some embodiments, the device comprises a membrane, either biological or solid state, containing the nanopore held between two reservoirs, also referred to as cis and trans chambers (see, e.g., U.S. Pat. No. 6,627,067). A conduit for electron migration between the two chambers allows electrical contact of the two chambers, and a voltage bias between the two chambers drives translocation of the tag through the nanopores. A variation of this configuration is used in analysis of current flow through nanopores, as described in U.S. Pat. Nos. 6,015,714 and 6,428,959; and Kasianowiscz et al., 1996, Proc Natl Acad Sci USA 93:13770-13773, the disclosures of which are incorporated herein by reference.

Variations of above the device are disclosed in U.S. application publication no. 2003/0141189. A pair of nanoelectrodes, fabricated by electrodeposition, is positioned on a substrate surface. The electrodes face each other and have a gap distance sufficient for passage of a single nucleic acid. An insulating material protects the nanoelectrodes, exposing only the tips of the nanoelectrodes for the detection of the nucleic acid. The insulating material and nanoelectrodes separate a chamber serving as a sample reservoir and a chamber to which the polymer is delivered by translocation. Cathode and anode electrodes provide an electrophoresis electric field for driving the tag from the sample chamber to the delivery chamber.

The current bias used to drive the analyte through the nanopore can be generated by applying an electric field directed through the nanopore. In some embodiments, the electric field is a constant voltage or constant current bias. In other embodiments, the movement of the tag is controlled through a pulsed operation of the electrophoresis electric field parameters (see, e.g., U.S. Patent Application No. 2003/141189 and U.S. Pat. No. 6,627,067). Pulses of current may provide a method of precisely translocating one or only a few bases of an oligonucleotide tag for a defined time period through the pore and to briefly hold the tag within the pore, and thereby provide greater resolution of the electrical properties of the tag.

The nanopore devices may further comprise an electric or electromagnetic field for restricting the orientation of the analyte as it passes through the nanopore. This holding field can be used to decrease the movement of the analyte within the nanopore. In some embodiments, an electric field that is orthogonal to the direction of translocation is provided to restrict the movement of the tag molecule within the nanopore. This is illustrated in U.S. Application Publication No. 2003/0141189 through the use of two parallel conductive plates above and beneath the sample plate. These electrodes generate an electric field orthogonal to the direction of translocation of an analyte molecule, and thus holding the tag molecule to one of the sample plates. A negatively charged backbone of a DNA, or nucleic acid modified to have negative charges on one strand, will be oriented onto the anodic plate, thereby limiting the motion of the tag molecule.

In still other embodiments, controlling the position of the analyte is carried out by the method described in U.S. Application Publication No. 2004/0149580, which employs an electromagnetic field created in the nanopore via a series of electrodes positions near or on the nanopore. In these embodiments, one set of electrodes applies a direct current voltage and radio frequency potential while a second set of electrodes applies an opposite direct current voltage and a radio frequency potential that is phase shifted by 180 degrees with respect to the radio frequency potential generated by the first set of electrodes. This radio frequency quadrupole holds a charged particle (e.g., nucleic acid) in the center of the field (i.e., center of the pore).

In exemplary embodiments, the nanopore membrane may be a multilayer stack of conducting layers and dielectric layers, where an embedded conducting layer or conducting layer gates provides well-controlled and measurable electric field in and around the nanopore through which the tag translocates. In an aspect, the conducting layer may be graphene. Examples of stacked nanopore membranes are found in US20080187915 and US20140174927, for example.

It is understood that the nanopore may be located in a membrane, layer or other substrate, which terms have been used interchangeably to describe a two-dimensional substrate comprising a nanopore.

In certain embodiments, the nanopore may be formed as part of the assay process for detecting and/or determining concentration of an analyte using the nanopore. Specifically, a device for detecting and/or determining concentration of an analyte using a nanopore may initially be provided without a nanopore formed in a membrane or layer. The device may include a membrane separating two chambers on the opposite sides of the membrane (a cis and a trans chamber). The cis and the trans chambers may include a salt solution and may be connected to a source of electricity. When a nanopore is to be created in the membrane, a voltage is applied to the salt solution in the cis and trans chamber and conductance through the membrane measured. Prior to the creation of a nanopore, there is no or minimal current measured across the membrane. Following creation of a nanopore, the current measured across the membrane increases. The voltage may be applied for an amount of time sufficient to create a nanopore of the desired diameter. Following the creation of a nanopore, an analyte or tag may be translocated through the nanopore and the translocation event detected. In certain embodiments, the same salt solution may be used for nanopore creation as well as for detection of translocation of an analyte or tag through the nanopore. Any suitable salt solution may be utilized for nanopore creation and/or translocation of an analyte or tag through the nanopore. Any salt solution that does not damage the counting label can be used. Exemplary salt solutions include lithium chloride, potassium chloride, sodium chloride, calcium chloride, magnesium chloride and the like. The concentration of the salt solution may be selected based on the desired conductivity of the salt solution. In certain embodiments, the salt solution may have a concentration ranging from 1 mM to 10 M, e.g., 10 mM-10 M, 30 mM-10 M, 100 mM-10 M, 1 M-10 M, 10 mM-5 M, 10 mM-3 M, 10 mM-1 M, 30 mM-5 M, 30 mM-3 M, 30 mM-1 M, 100 mM-5 M, 100 mM-3 M, 100 mM-1 M, 500 mM-5 M, 500 mM-3 M, or 500 mM-1 M, such as, 10 mM, 30 mM, 100 mM, 500 mM, 1 M, 3 M, 5 M, or 10 M.

In some embodiments, the nanopore may become blocked, and the blocked nanopore is cleared by modulating the pattern of voltage applied by the electrodes across the nanopore layer or membrane. In some cases, a blocked nanopore is cleared by reversing polarity of the voltage across the nanopore layer or membrane. In some cases, a blocked nanopore is cleared by increasing the magnitude of the voltage applied across the nanopore layer or membrane. The increase in voltage may be transitory increase, lasting 10 seconds(s) or less, e.g., 8 s or less, 6 s or less, 5 s or less, 4 s or less, 3 s or less, 2 s or less, 1 s or less, 0.5 s or less, 0.4 s or less, 0.3 s or less, 0.2 s or less, including 0.1 s or less.

In some embodiments, the sample analysis region comprises a quintenary zone. In some embodiments, the quintenary zone is a hydrophilic liquid well. In some embodiments, the quintenary zone is a hydrophilic liquid reservoir. When the quintenary zone is a hydrophilic liquid well, the hydrophilic liquid is directly added to the well after the microparticles or microparticles and assisting particles are added to the sample detection zone. In some embodiments, when the sample detection zone comprises wells or microwells, the hydrophilic liquid is added after the microparticles are seeded into the wells or microwells. When the quintenary zone is a hydrophilic liquid reservoir, the hydrophilic liquid may be pulled from a reservoir though a pump, gravity, suction, etc. In some embodiments, the hydrophilic liquid is a substrate solution. The substrate solution may be any substrate solution that reacts with a specific binding member (e.g., the second specific binding member or the detectably labeled second specific binding member) to produce a detectable signal.

In some embodiments, the quaternary zone comprises one or more substrate retention features. The substrate retention features of the present disclosure may be a range of different features and may be any combination of substrate features described herein. In some embodiments, the substrate retention feature is a first, a second, a third and a fourth substrate retention feature wherein each substrate retention feature is quarter circle shaped and protrudes into the quaternary zone such as depicted in FIG. 33G. In some embodiments, the substrate retention feature is centrally located on the right end of the sample analysis region, is quarter circle shaped, and protrudes from the top substrate or bottom substrate as depicted in FIG. 33H. In some embodiments, the substrate retention feature is located on the right end of the sample analysis region, is horseshoe shaped with tapered edges, and protrudes from the top substrate or bottom substrate as depicted in FIG. 33H. In some embodiments, the substrate retention feature is a first substrate retention feature located on the right end of the sample analysis region, is horseshoe shaped without tapered edges, and is concentric with a second substrate retention feature that is horseshoe shaped without tapered edges wherein the first and the second substrate retention protrude from the top or bottom substrates such as depicted in FIG. 33I.

In some embodiments, the substrate retention feature is a first, a second, a third and a fourth substrate retention feature wherein the first through fourth substrate retention features are grooves on the top or bottom substrates, are located on the right end of the sample analysis region, and are concentric with each other such as depicted in FIG. 33J. In some embodiments, the substrate retention feature is located on the right end of the sample analysis region, is horseshoe shaped without tapered ends, and protrudes from the top or bottom substrate as depicted in FIG. 33K. In some embodiments, the substrate retention feature is a first and a second substrate retention feature wherein the first and second substrate retention features are located prior to the right end of the sample analysis region, are rectangular shaped, the first substrate retention is opposite the second substrate retention feature, and the first and second substrate retention feature protrude from the top or bottom substrate such as depicted in FIG. 33L. In some embodiments, the substrate retention feature is a first and a second substrate retention feature wherein the first and second substrate retention features are located prior to the right end of the sample analysis region, are circular shaped, the first substrate retention is opposite the second substrate retention feature, and the first and second substrate retention feature protrude from the top or bottom substrate such as depicted in FIG. 33M.

In some embodiments, the sample analysis region comprises a quinary zone. In some embodiments, the quinary zone is a hydrophobic liquid well. In some embodiments, the quinary zone is a hydrophobic liquid reservoir. When the quinary zone is a hydrophobic liquid well, the hydrophobic liquid is directly added to the well after the microparticles or microparticles and assisting particles are added to the sample detection zone and following the addition of the hydrophilic liquid. In some embodiments, when the sample detection zone comprises wells or microwells, the hydrophobic liquid is added after the microparticles or microparticles and assisting particles are seeded into the wells or microwells following hydrophilic liquid addition to seal the wells or microwells for detection. When the quinary zone is a hydrophobic liquid reservoir, the hydrophobic liquid may be pulled from a reservoir though a pump, gravity, suction, etc. In some embodiments, the hydrophobic liquid is oil. The oil may be any oil deemed useful. In certain cases, the hydrophobic liquid is selected based on its low affinity for water to decrease mixing of the hydrophobic liquid with the substrate solution. In certain cases, the hydrophobic liquid is an oil. In certain cases, the hydrophobic liquid is 3M FC-40 oil, a hydrocarbon oil, a vegetable oil, or silicone liquids (e.g., a silicone oil). In certain cases, the oil is a fluorocarbon oil. In certain cases, the oil is Novec 7500, FC-40, or Galden HT200.

In some embodiments, the quinary zone comprises a barrier feature. In some embodiments, the barrier feature is on the left end of the sample analysis region, is half circle shaped, and protrudes from the top or the bottom substrate.

### 3. Reagent Delivery Cartridge

The present disclosure also provides an optional regent delivery device. The reagent delivery device contains a sample collection portion, a reagent cartridge, a frame, a seal and an integrated sample processing device.

In some embodiments, one or more reagents are added to the device using the reagent delivery device. In some embodiments, one or more reagents are added to the device by individually adding the one or more reagents to the device. In some embodiments, one or more reagents are added to the device using bulk reagent delivery. “Bulk reagent delivery” as used herein refers to the addition of reagents to the device using a storage container containing a reagent at a volume that exceeds the volume of the reagent necessary to perform any of the assays disclosed herein once. For instance, the storage container contains a sufficient volume of the reagent such that two or more of any of the assays disclosed may be performed without refiling the storage container. The storage container may sufficient volume of the reagent such that 2 or more, 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 6000 or more, 7000 or more, 8000 or more, 9000 or more, or 10000 or more to of any of the assays disclosed may be performed without refiling the storage container.

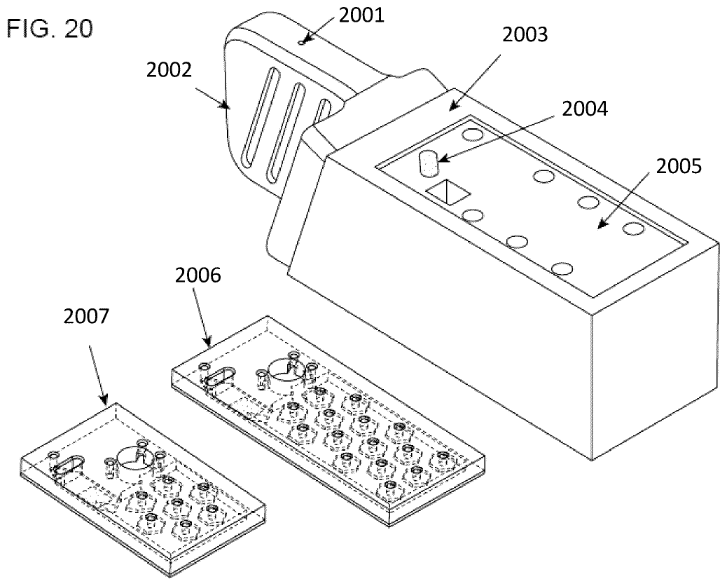

FIG. 20 depicts an illustration of a partially assembled reagent delivery device. The reagent delivery contains a sample collection portion 2002. The sample collection portion contains an opening 2001 that is connected to a capillary portion that collects the sample. The sample collection portion is connected to the frame 2003. The frame holds the reagent cartridge 2005. In the reagent cartridge is an independent plunger 2004. The reagent delivery device connects to exemplary sample processing devices 2006 or 2007. The reagent delivery device may connect to any of the devices disclosed herein.

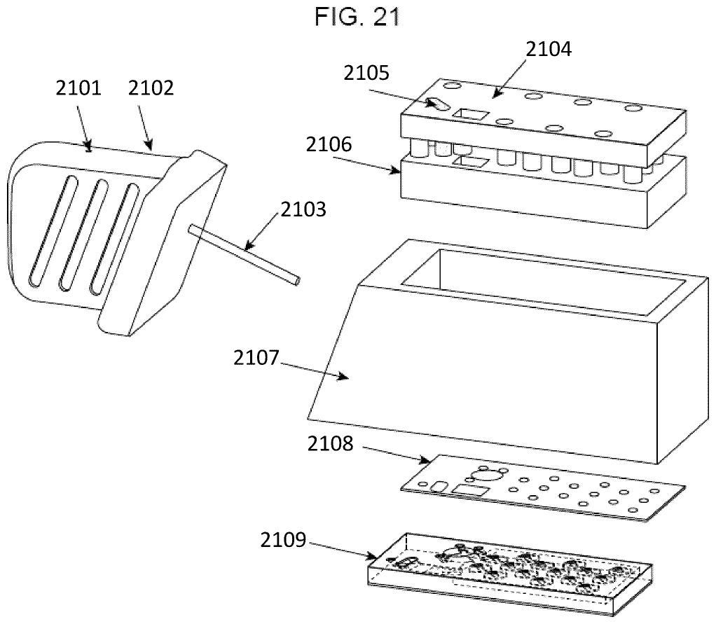

FIG. 21 depicts an illustration of a deconstructed view of the reagent delivery device. The reagent delivery device contains a sample collection portion 2102. The sample collection portion is connected a hollow capillary portion 2103. The capillary portion may be contacted to a sample to collect sample in the hollow capillary portion 2103. The reagent cartridge contains a plunger portion 2104, an independent plunger 2105 and a reagent containing portion 2106. The reagent cartridge connects to the interior of the frame 2107. The sample collection portion 2102 connects to exterior of the frame 2107. A seal 2108 is connected to a sample processing device 2109. The seal 2108 and sample processing portion are connected to the bottom interior of the frame 2107.

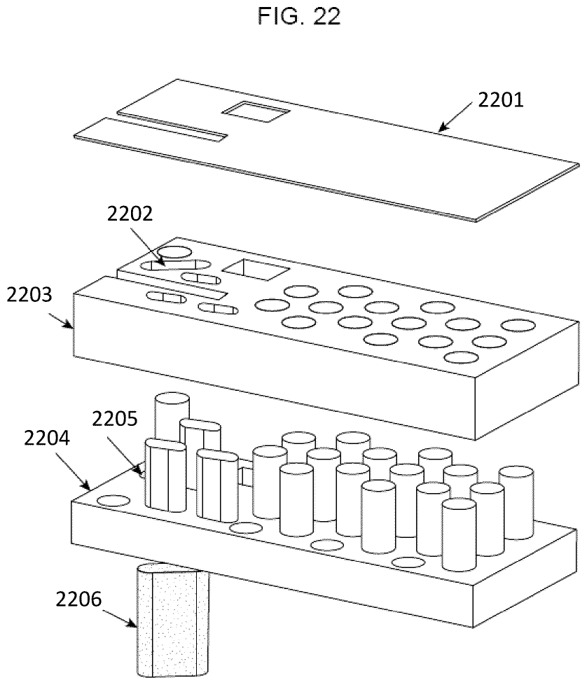

FIG. 22 discloses an exemplary embodiment of an upside down view of the reagent cartridge. The reagent cartridge contains a seal 2201 that is connected to the reagent containing portion 2203. The seal 2201 prevents the reagents from leaving the reagent containing portion 2203. The plunger portion contains one or more plungers that are physically connected such that depression of the plunger portion depresses the one or more plungers. In some embodiments, the plunger portion contains both independent and connected plungers. The reagent cartridge contains an independent plunger that fits into openings 2205 and 2202. In some embodiments, the independent plunger dispenses a hydrophobic liquid from the reagent containing portion 2203.

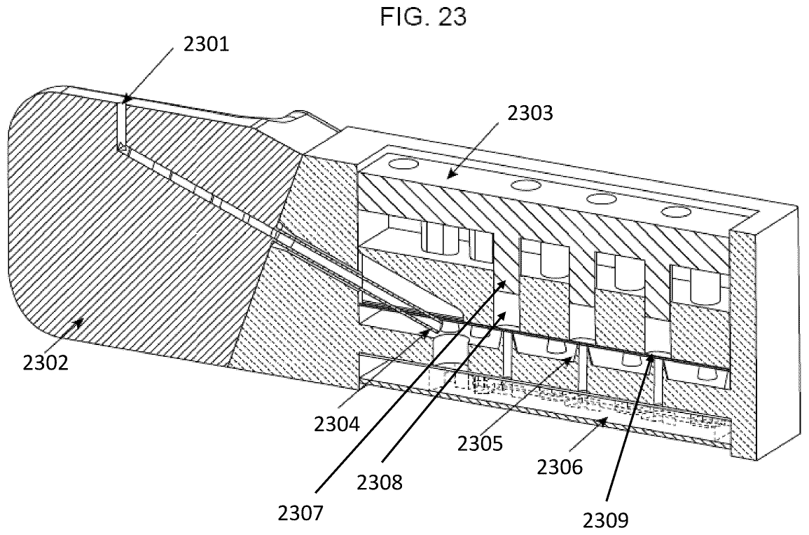

FIG. 23 depicts an illustration of a cross-section of the reagent delivery device in a non-activated state. The reagent delivery device has a sample collection portion 2202 physically connected to the frame. The sample collection portion contains an opening 2201 that is connected to the capillary portion. When the capillary portion contains a sample and pressure is applied to the device, the sample is delivered to a variable or fixed primary zone of the sample processing device. The reagent containing portion contains reagents 2208 that are dispensed into primary, secondary, quaternary and/or quinary zones following depression of the plunger portion. The seal 2309 of the reagent cartridge becomes pierced by film piercing features 2205 and allow reagents to be delivered to the sample processing device.

FIG. 24 depicts an illustration of a cross-section of the reagent delivery device in an activated state. In the activated state, the plunger portion 2403 is depressed and the film piercing features 2407 thereby releasing reagents into the sample processing device 2406.

FIG. 25 depicts an alternative design of the reagent cartridge. In place of the film piercing features disclosed in FIGS. 23 and 24, there are film piercing features connected directed to the plunger portion and are present in the fluid chamber 2507. Upon depression, the film piercing features 2504 pierce the seal 2505 and allow reagents to be dispensed in the sample processing device.

### 4. Methods and Devices for Mixing on or Off the Device

The present disclosure provides methods and devices for mixing the fluids in the device. The fluids for use in the device may be mixed in the device itself or prior to addition to the device.

In some embodiments, the fluids in the device are mixed by vibrating the entire device. In these embodiments, the vibration source contacts the device and vibrates the entire device. In some embodiments, the vibration is in a vertical motion. In some embodiments, the vibration is in a horizontal motion. The vibration source may be a range of different sources including, without limitation, a voice coil, a vibration motor, a piezo actuator, a motor, a gas pump, etc.

In some embodiments, the fluids in the are mixed by vibrating only the top substrate. The vibration is achieved by contacting the top substrate with the vibration device. In some embodiments the entire top substrate is vibrated. In some embodiments, the entire top substrate is vibrated vertical. In some embodiments, a portion of the top substrate is vibrated. In some embodiments, the portion of the top substrate is a primary zone. In some embodiments, the primary zone is in a sample mixing zone.

FIG. 10 discloses an illustration of an exemplary sample mixing zone and method of mixing. In this embodiment, the sample mixing zone comprises a fixed primary zone 1006 containing a sample 1005. The sample is added through the opening 1004 in the fixed primary zone. In some embodiments, the fixed primary zone may contain two or more openings. In some embodiments, the fixed primary zone may contain three or more openings. The fixed primary zone 1006 contains a hooked portion 1002. The hooked portion is capable of joining with a vibration source 1001. The vibration source provides vertical motion 1003 that compresses and decompresses the sample 1005. The compression and decompression of the sample 1005 results in the mixing of the sample. The fixed primary zone 1006 is a cantilever in that only the portion opposite the opening 1004 is attached to the top substrate. All other regions around the fixed primary zone are detached from the top substrate.

FIG. 32A discloses an alternative embodiment to FIG. 10 where the sample mixing zone does not contain a hooked portion. In this embodiment, the sample mixing zone comprises a fixed primary zone 3200. The sample is added through the opening 3201 in the fixed primary zone In this embodiment, the fixed primary zone contains two openings. In some embodiments, the fixed primary zone one or more openings. In some embodiments, the fixed primary zone contains three or more openings. The fixed primary zone 3200 does not contain a hooked portion. A vibration source comes in contact with the end 3202 of the fixed primary zone. The vibration source provides vertical motion that compresses and decompresses the sample. The compression and decompression of the sample results in the mixing of the sample. The fixed primary zone 3200 is a cantilever in that only the portion opposite the end 3202 is attached to the top substrate. All other regions around the fixed primary zone are detached from the top substrate.

In some embodiments, the fluids in the device are mixed by vibrating only the bottom substrate. The vibration is achieved by contacting the bottom substrate with the vibration device. In some embodiments, the bottom substrate is vibrated. In some embodiments, the entire bottom substrate is vibrated vertically. In some embodiments, a portion of the bottom substrate is vibrated. In some embodiments, the portion of the bottom substrate is a primary zone. In some embodiments, the primary zone is in a sample mixing zone.

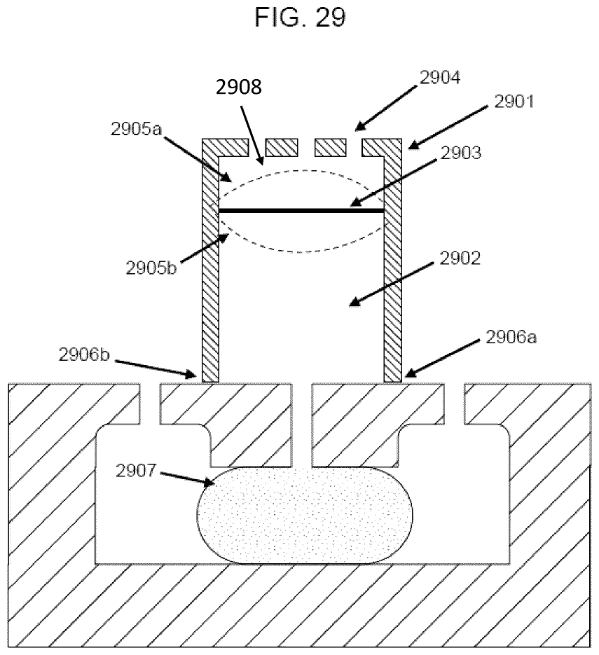

FIG. 29 discloses an illustration of an exemplary pressure sample mixing device and method. In this embodiment, the sample mixing device 2901 contacts a fixed or variable primary zone at contact point 2906a and 2906b. In some embodiments, the sample mixing device 2901 contacts a fixed or variable primary zone at contact point 2906a and 2906b with a suction cup. In some embodiments, the sample mixing device 2901 contacts a fixed or variable primary zone at contact point 2906a and 2906b with a gasket. In some embodiments, the sample mixing device 2901 contacts a fixed or variable primary zone at contact point 2906a and 2906b in the absence of a suction cup or gasket. The sample mixing device 2901 contains a chamber 2902 (i.e. a bottom portion) which comprises air and a top portion 2908 separated by a diaphragm. The chamber has an open bottom. Above the chamber 2902 that contains air is a diaphragm 2903. The diaphragm may be moved or oscillated by a deflection amount 2905a and 2905b that compresses the air in the chamber 2902. The compression in the air results in movement in the fluid 2907 in the primary zone thereby mixing the fluid. The sample mixing device 2901 comprises one or more openings 2904 that allow pressure relief in the sample mixing device 2901 when the diaphragm 2903 is moving or oscillating.

The diaphragm of the sample mixing device may be made of a range of different materials including, without limitation, piezoelectric material, metal, a compliant material that deforms under heat or pressure, etc. The diaphragm of the sample mixing device may be moved or oscillated in a number of different ways. In some embodiments, the diaphragm is made of a piezoelectric material. In some embodiments, the piezoelectric material is moved or oscillated by applying an electric current to the piezoelectric material. In some embodiments, the diaphragm is metal. In some embodiments, the metal is moved or oscillated by turning a magnetic field on and off. In some embodiments, the diaphragm is made of a compliant material that deforms under heat or pressure. In some embodiments, the compliant material is moved by apply air pressure to the compliant material. In some embodiments, the compliant material is moved by applying heat to the compliant material. In some embodiments, the compliant material is moved by applying acoustic waves to the compliant material. In some embodiments, the compliant material is moved by applying a mechanical force to the compliant material.

In some embodiments, the fluid in the fixed or variable primary zone is mixed in the absence of a sample mixing device contacting the device. In some embodiments, the fluid is mixed by applying pulsed air in the opening in the fixed or variable primary zone. In some embodiments, the fluid is mixed by applying jetted air in the opening in the fixed or variable primary zone. In some embodiments, the fluid is mixed by applying constant acoustic waves in the opening in the fixed or variable primary zone. In some embodiments, the fluid is mixed by applying pulsed acoustic waves in the opening in the fixed or variable primary zone.

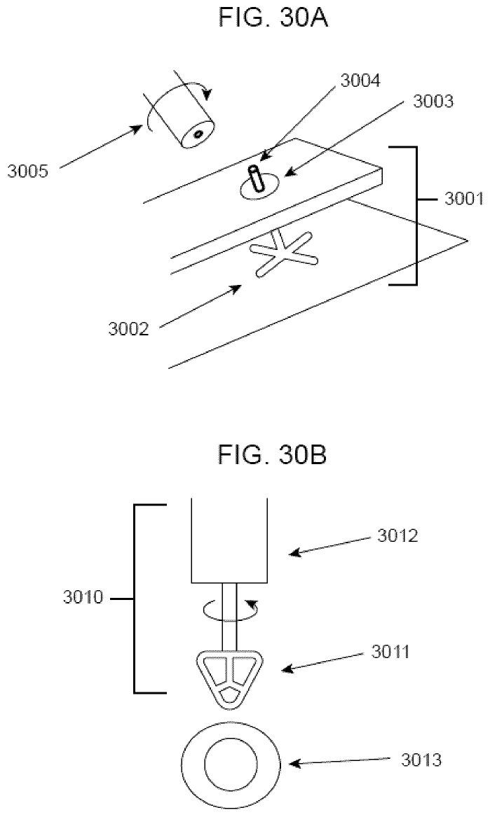

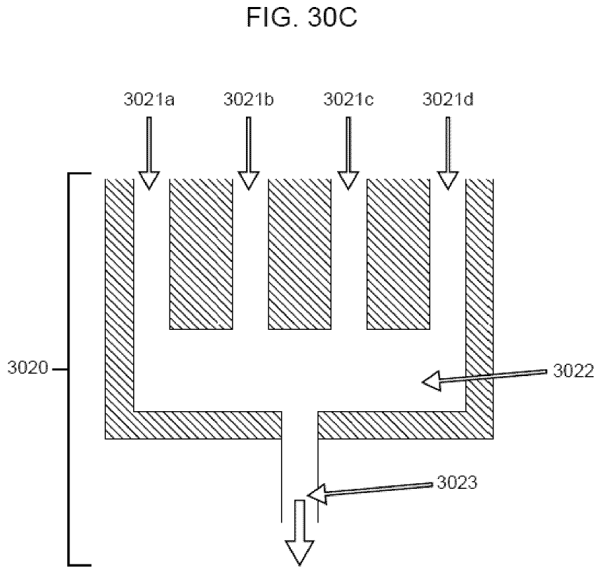

FIG. 30A discloses an illustration of an exemplary impeller sample mixing device and method. An impeller element 3002 is placed or is already present in a fixed or variable primary zone 3001 in the opening 3003 in the fixed or variable primary zone 3001. The impeller element 3002 attaches to a rotation device 3005 which rotates the impeller element thereby mixing the fluid in the fixed or variable primary zone. The impeller element may have foldable blades such that when the impeller element is pushed into the opening 3003 of the fixed or variable primary zone the blades of the impeller element fold towards to shaft of the impeller element so that the impeller elements fits through the opening 3003 of the fixed or variable primary zone.

FIG. 30B discloses an illustration of an exemplary rotating sample mixing device and method. A rotating nozzle 3012 of the rotating sample mixing device 3010 is placed in the opening 3013 of a fixed or variable primary zone. The rotating nozzle 3012 has an inverted Y-shaped rib structure that when rotated by the rotation device 3010 mixes the fluid present in the fixed or variable primary zone. The rotating nozzle may have a range of different size. For instance, the rotating nozzle may have 0.5×, 0.6×, 0.7×, 0.8×, or 0.9× the size of the opening in the fixed or variable primary zone. The rotating nozzle does not exceed 0.9× the size of the opening in the fixed or variable primary zone.

FIG. 30C discloses an illustration of an exemplary swirling sample mixing device and method. The swirling sample mixing device 3020 comprises fluid addition ports 3021a-d, a swirling chamber 3022, and a mixed fluid exit port 3023. The swirling chamber 3022 comprises a series of inclined rib structures that are oriented radially around the chamber such that the inclined rib structures facilitate swirling of fluids added to the swirling chamber in the absence of mechanical or electrical intervention. Fluids are added to one or more of the fluid addition ports 3021a-d. The swirling sample mixing device may contain more or less than the 4 sample addition ports but contains at least two sample addition ports. For instance, the swirling sample mixing device 3020 may contain two or more, three or more, four or more, five or more, six or more, seven or more, eight or more, nine or more, or ten or more sample addition port. In each sample addition port 3021a-d, a fluid is added (e.g., sample contain an analyte, fluid containing microparticles, fluid containing assisting particles, wash buffer, lysis buffer, dilution buffer, etc.). The fluids travel through the fluid addition ports 3021a-d into the swirling chamber 3022 where the fluids mix together. Following mixing, the mixed fluid is dispensed through the mixed fluid exit port 302. The mixed fluid is dispensed into the opening of a fixed or variable primary zone.

### 5. Methods of Detecting a Target Analyte in a Sample

The present provides methods of detecting a target analyte in a sample using the devices disclosed herein. The samples that may be used with the methods will be discussed first followed by the types of analytes followed by the specific types of assay (e.g., digital analysis, immunoassays, nucleic acid analysis, clinical chemistry, etc.) to be used in the methods.

### I. Samples

Aspects of the present disclosure devices that may be used to process and analyze different analytes or different types of analytes present in a biological sample and methods of use thereof. In some embodiments, the devices of the present disclosure comprise fixed or variable primary zones where the sample may be deposited. In order to analyze a sample, more specifically analytes in a sample, the sample is derived from one or more of various sample sources described in this section.

### Sample Type

As used herein, “sample”, “test sample”, or “biological sample” refers to a sample containing or suspected of containing an analyte. For example, International Publication No. WO 2016/161400 are incorporated by reference herein and samples of the present disclosure are further described below.

In some embodiments, a sample of the present disclosure is derived from any suitable source. In other embodiments, the sample comprise a liquid, fluent particulate solid, or fluid suspension of solid particles. In certain embodiments, the sample may be a liquid sample or a liquid extract of a solid sample.

In some cases, the sample may be processed prior to the analysis described herein. For example, the sample may be separated or purified from its source prior to analysis; however, in certain embodiments, an unprocessed sample containing the analyte may be assayed directly.

In some embodiments, the source of the analyte molecule may be synthetic (e.g., produced in a laboratory), the environment (e.g., air, soil, etc.), fluid samples, e.g., water supplies, etc.), an animal, e.g., a mammal, a plant, or any combination thereof.

In some embodiments, a sample as the source of an analyte is a human bodily substance. The human bodily substance may be a liquid sample or a liquid extract of a solid sample. Non-limiting embodiments of the human bodily substance is bodily fluid, blood, serum, plasma, urine, saliva, sweat, sputum, semen, mucus, lacrimal fluid, tears, dermal fluid, lymph fluid, amniotic fluid, interstitial fluid, intestinal fluid, gastrointestinal fluid, lung lavage, spinal fluid, cerebrospinal fluid, feces, nasal mucus, virginal discharge, tissue, organ, or like. In some embodiments, tissues may include, but are not limited to, skeletal muscle tissue, liver tissue, lung tissue, kidney tissue, myocardial tissue, brain tissue, bone marrow, cervix tissue, skin, etc. In certain cases, the source of the sample may be an organ or tissue, such as a biopsy sample, which may be solubilized by tissue disintegration/cell lysis.

In certain embodiments, a sample of the present disclosure is whole blood. Samples for hematology are typically whole blood. The whole blood sample consists of red blood cells, white blood cells, and platelets suspended in a protective yellow liquid known as plasma. In some embodiments, samples for immunoassays and clinical chemistry assays are typically serum or plasma. In some embodiments, the whole blood sample is obtained from a subject. In some embodiments, the subject is a living subject, including an animal and a human.

In certain embodiments, a sample of the present disclosure is venous blood. As used herein, the term “venous blood” refers to a sample of blood taken from a certain vein and checked for specific substances released by nearby organs and tissues. A higher-than-normal amount of a substance can be a sign of disease in the organ or tissue. In some embodiments, venous blood is collected by a venous blood sampling process. For example, in venous blood sampling, a needle is inserted into a vein to collect a sample of blood for testing.

In certain embodiments, a sample of the present disclosure is capillary blood. As used herein, “capillary blood”, or “capillary sample” refers to a blood sample collected by pricking the skin. Capillary blood is generally obtained by pricking a finger in adults and a heel in infants and small children. Capillaries are tiny blood vessels near the surface of the skin. Capillary plasma typically contains higher concentrations of proteins, calcium and chloride, and lower levels of potassium, sodium, and urea nitrogen compared to venous plasma.

In certain embodiments, a sample of the present disclosure is plasma. As used herein, the term “plasma” refers to the colorless fluid part of blood, lymph, or milk, in which corpuscles or fat globules are suspended. As such, plasma is the blood's liquid component and is made up of water, proteins, waste products, minerals, clotting factors, immunoglobulins, carbon dioxide and hormones. The method for separating plasma from blood is well known in the art. In exemplary embodiments, plasma is produced when whole blood is collected in tubes that are treated with an anticoagulant. The blood does not clot in the plasma tube, thereby the cells are removed by centrifugation. The supernatant, designated plasma is carefully removed from the cell pellet

In certain embodiments, a sample of the present disclosure is serum. As used herein, the term “serum” refers to the watery, clear portion of an animal fluid or plant sap. As used herein, the term “blood serum” refers to an amber-colored, protein-rich liquid that separates out when blood coagulates. In certain embodiments, serum includes, but not limited to, blood serum, serous (or serosal) fluid secreted by the serous glands, and plant sap. The method for separating serum from blood is well known in the art. In exemplary embodiments, the blood serum is collected after whole blood is allowed to clot. The clot is removed by centrifugation, and the resulting supernatant, designated serum, is carefully removed.

In certain embodiments, a sample of the present disclosure is a cerebrospinal fluid. The term “cerebrospinal fluid (CSF)” refers to a clear fluid that surrounds and protects the brain and spinal cord. The analysis for cerebrospinal fluid may look for proteins, sugar (glucose), and other substances. The method for collecting cerebrospinal fluid is well known in the art. In exemplary embodiments, cerebrospinal fluid is usually obtained through a lumbar puncture (spinal tap). During the procedure, a needle is inserted usually between the 3rd and 4th lumbar vertebrae and the CSF fluid is collected for testing.

In certain embodiments, a sample of the present disclosure is saliva. As used herein, the term “saliva” refers to watery liquid secreted into the mouth by glands, providing lubrication for chewing and swallowing, and aiding digestion. Saliva consists of 99% water and 1% protein and salts. The method of collecting saliva is well known in the art. In some embodiments, saliva sample can be refrigerated for up to a week before it needs to be added to the stabilizing fluid in the tube.

In certain embodiments, a sample of the present disclosure is urine. As used herein, the term “urine” refers to a watery, typically yellowish fluid stored in the bladder and discharged through the urethra. Urine is one of the body's chief means of eliminating excess water and salt, and also contains nitrogen compounds such as urea and other waste substances removed from the blood by the kidneys. Collecting a urine sample is well known in the art. In exemplary embodiments, either a “first-catch” or a “mid-stream” sample of urine is collected in a completely sterile container. The first-catch urine sample is the first part of the urine that comes out. The mid-stream urine is for reducing the risk of the sample being contaminated with bacteria from hands, or the skin around the urethra or the tube that carries urine out of the body. In some embodiments, the collected urine sample may be stored in a fridge at 4° C. less than 24 hours in a sealed plastic bag. In certain embodiments, the urine sample is used for infections such as urinary tract infection (UTI), some sexually transmitted infections (STIs) such as chlamydia in men, or kidney damage, such as ACR test.

In certain embodiments, a sample of the present disclosure is interstitial fluid. As used herein, the expression “interstitial fluid (ISF)”, “lymph”, or “tissue fluid” refers to clear fluid that occupies the space between the cells in the body or fluid found in the spaces around cells. It comes from substances that leak out of blood capillaries. Interstitial fluid helps bring oxygen and nutrients to cells and to remove waste products from them. As new interstitial fluid is made, it replaces older fluid, which drains towards lymph vessels. The method for collecting interstitial fluid is well known in the art. In one embodiment, ISF can typically be collected from skin using suction blisters by applying suction to skin at elevated temperature for up to 1 hr to create blisters filled with ISF.

In certain embodiments, a sample of the present disclosure is intestinal fluid. Intestinal fluid or gastrointestinal fluid contains, for example electrolytes, bile salts, lipids and lipid digestion products, cholesterol, proteins, enzymes plus other components and may also vary depending upon the anatomical location (stomach vs small intestine vs colon). The method of collecting intestinal fluid samples is well known in the art. In certain embodiments, the intestinal fluid can be collected through a nasojejunal tube and be made into capsules using the freeze-dried powder method.

In certain embodiments, a sample of the present disclosure is a sample collected from nasal swabs. In certain embodiments, a sample of the present disclosure is a sample collected from throat swabs. In certain embodiments, a sample of the present disclosure is a sample collected from vaginal swabs. Nasal swabs, throat swabs, and vaginal swabs are well known in the art.

In certain embodiments, a sample includes respiratory specimen. For example, the respiratory specimen includes, but not is limited to, nasal swab, throat swab, sputum, tracheal/bronchial secretion, and bronchial lavage fluid. In some embodiments, respiratory sampling includes upper respiratory materials and lower respiratory secretions. In some cases, the upper respiratory materials comprise nasal swab, throat swab, and the like. In other cases, the lower respiratory secretions comprise sputum, tracheal/bronchial secretion, bronchoalveolar lavage fluid, and the like. In some embodiments, the sputum is collected by well known process in the art. For example, collecting sputum follows the steps of i) taking a very deep breath and holding the air for 5 seconds; ii) slowly breathing out; iii) taking another deep breath and coughing hard until some sputum coming up into mouth; iv) spiting the sputum into a sample container. In some embodiments, tracheal/bronchial secretion is collected by inserting suction catheter as deeply as possible and aspirating secretion, which is well known in the art. In some embodiments, bronchoalveolar lavage fluid is collected by use of bronchoscopy, which is well known in the art.

In some embodiments, a sample includes any tissue obtained from a subject. In other embodiments, a sample includes any cell obtained from a subject. The subject is any living subject including a human. In some embodiments, tissues may include, but are not limited to skeletal muscle tissue, liver tissue, heart tissue, lung tissue, pancreas tissue, adipose tissue, stomach tissue, gastrointestinal tract tissue, colon tissue, kidney tissue, myocardial tissue, brain tissue, breast tissue, nerve tissue, bone marrow, cervix tissue, skin, etc. In some embodiments, cells may include, but are not limited to skeletal muscle cells, liver cells, heart cells, lung cells, pancreas cells, adipose cells, stomach cells, gastrointestinal tract cells, colon cells, kidney cells, myocardial cells, brain cells, breast cells, nerve cells, bone marrow cells, cervix cells, skin cells, etc. In some cases, the sample is tumor or cancer cells. For example, the sample includes, but is not limited to, brain cancer cells, liver cancer cells, pancreas cancer cells, lung cancer cells, breast cancer cells, kidney cancer cells, metastatic cancer cells, ovarian cancer cells, colorectal cancer cells, bladder cancer cells, thyroid cancer cells, lymphoma cells, cervical cancer cells, gynecologic cancer cells, head and neck cancer cells, mesothelioma cells, myeloma cells, skin cancer cells, prostate cancer cells, uterine cancer cells, vaginal and vulvar cancer cells, and the like. In certain cases, the source of the sample may be an organ or tissue, such as a biopsy sample, which may be solubilized by tissue disintegration/cell lysis. In certain embodiments, a sample may be processed prior to performing immunoassay on the sample. For example, the sample may be concentrated, diluted, purified, amplified, etc.

### II. Target Analytes

In some embodiments, one or more analytes in a sample may be measured, detected, or assessed by the device of the present disclosure. The sample may be any sample containing or suspected of containing an analyte. As used herein, “analyte”, “target analyte”, and “analyte” are used interchangeably and refer to the analyte being measured in the devices disclosed herein. Examples of analytes provided herein are for illustrative purposes and are not intended to limit the scope of the present disclosure. For example, Patent Publication No. WO2018/161402 is incorporated by reference herein and analytes are further described below.

In some embodiments, but not by way of limitation, the analyte may be a pathogen, a prion protein, a cancer cell, a blood component, or a biomolecule. In some cases, the pathogen is, but not limited to, a virus, a bacterium, a fungus, or a protozoan. In some cases, the prion protein may arise from a sporadic prion disease, a genetic prion disease, or an acquired prion disease. In some cases, the cancer cell may be a cancer cell from a tumor or a circulating tumor cell. In some cases, the blood component may be red blood cells, white blood cells, platelets, or proteins found in the blood. In some cases, a biomolecule may be a metabolite, a macromolecule, a protein, or a chemical compound. Any combination of analytes may be measured by the assays of the methods and systems of the present disclosure.

In some embodiments, one or more analytes may be a cell, such as, circulating tumor cell. In other embodiments, the analyte is a biological cell (e.g., mammalian, avian, reptilian, other vertebrate, insect, yeast, bacterial, cell, etc.). In other embodiments, the analyte may be an infectious agent, such as a bacterium (e.g., Mycobacterium tuberculosis, Staphylococcus aureus, Shigella dysenteriae, Escherichia coli O157:H7, Campylobacter jejuni, Listeria monocytogenes, Pseudomonas aeruginosa, Salmonella O8, and Salmonella enteritidis), virus (e.g., retroviruses (such as HIV), herpesviruses, adenoviruses, lentiviruses, Filoviruses (e.g., West Nile, Ebola, and Zika viruses), hepatitis viruses (e.g., A, B, C, D, and E); HPV, Parvovirus, etc.), a parasite, or fungal spores.

In exemplary embodiments, one or more analytes are tumor or cancer cells. In some cases, a cancer cell may be directly detected, e.g., a nucleic acid or an antigen specific to the cancer cell is detected. In some cases, the presence of a cancer cell may be detected by a change or mutation in a nucleic acid sequence of the cancer cell, including, but not limited to, a SNP, an insertion, a deletion, a chromosome translocation, or gene amplification. In some cases, a cancer cell may be detected by detecting the presence of tumor or cancer markers associated with the cancer cell. In some cases, a cancer cell may be detected by detecting the expression of receptors associated with a cancer cell. In some cases, a cancer cell may be indirectly detected, e.g., metabolic markers associated with the cancer cell can indicate the presence of the cancer cell.

For example, types of cancer cells that may be detected by assays of the present disclosure include, but are not limited to, carcinoma cells, leukemia cells, lymphoma cells, myeloma cells, sarcoma cells, central nervous system cancer cells, and mesothelioma cells. Specific types of cancer include, but are not limited to, Bone Cancer (includes Ewing Sarcoma and Osteosarcoma and Malignant Fibrous Histiocytoma), Brain Tumors, Breast Cancer, Cervical cancer, Colorectal Cancer, Endometrial Cancer (Uterine Cancer), Esophageal Cancer, Head and Neck Cancer, Hepatocellular (Liver) Cancer, Hodgkin Lymphoma, Kidney (Renal Cell) Cancer, gynecologic cancer cells, vaginal and vulvar cancer cells, Leukemia, Lung Cancer (Non-Small Cell, Small Cell, Pleuropulmonary Blastoma, Pulmonary Inflammatory Myofibroblastic Tumor, and Tracheobronchial Tumor), Lymphoma, Melanoma, Multiple Myeloma/Plasma Cell Neoplasms, Neuroblastoma, Non-Hodgkin Lymphoma, Ovarian Cancer, Pancreatic Cancer, Prostate Cancer, Skin Cancer, Testicular Cancer, Thyroid Cancer. Markers of cancer include, but are not limited to, ALK gene rearrangements and overexpression, Alpha-fetoprotein (AFP), B-cell immunoglobulin gene rearrangement, BCL2 gene rearrangement, Beta-2-microglobulin (B2M), Beta-human chorionic gonadotropin (Beta-hCG), Bladder Tumor Antigen (BTA), BRCA1 and BRCA2 gene mutations, BCR-ABL fusion gene (Philadelphia chromosome), RAF V600 mutations, C-kit/CD117, CA15-3/CA27.29, CA19-9, CA-125, CA 27.29, Calcitonin, Carcinoembryonic antigen (CEA), CD19, CD20, CD22, CD25, CD30, CD33, Chromogranin A (CgA), Chromosome 17p deletion, Chromosomes 3, 7, 17, and 9p21, Circulating tumor cells of epithelial origin (CELLSEARCH), Cytokeratin fragment 21-1, Cyclin D1 (CCND1) gene rearrangement or expression, Des-gamma-carboxy prothrombin (DCP), DPD gene mutation, EGFR gene mutation, Estrogen receptor (ER)/progesterone receptor (PR), FGFR2 and FGFR3 gene mutations, Fibrin/fibrinogen, FLT3 gene mutations, Gastrin, HE4, HER2/neu gene amplification or protein overexpression, 5-HIAA, IDH1 and IDH2 gene mutations, Immunoglobulins, IRF4 gene rearrangement, JAK2 gene mutation, KRAS gene mutation, Lactate dehydrogenase, Microsatellite instability (MSI) and/or mismatch repair deficient (dMMR), MYC gene expression, MYD88 gene mutation, Myeloperoxidase (MPO), Neuron-specific enolase (NSE), NTRK gene fusion, Nuclear matrix protein 22, PCA3 mRNA, PML/RARα fusion gene, Prostatic Acid Phosphatase (PAP), Programmed death ligand 1 (PD-L1), Prostate-specific antigen (PSA), ROS1 gene rearrangement, Soluble mesothelin-related peptides (SMRP), Somatostatin receptor, T-cell receptor gene rearrangement, Terminal transferase (TdT), Thiopurine S-methyltransferase (TPMT) enzyme activity or TPMT genetic test, Thyroglobulin, UGT1A1*28 variant homozygosity, Urine catecholamines: VMA and HVA, Urokinase plasminogen activator (uPA) and plasminogen activator inhibitor (PAI-1), FoundationOne CDx (F1CDx) genomic test, Guardant360 CDx genomic test, 5-Protein signature (OVA1), 17-Gene signature (Oncotype DX GPS test), 21-Gene signature (Oncotype DX), 46-Gene signature (Prolaris), 70-Gene signature (Mammaprint).

Furthermore, types of cancer cells that may be detected by assays of the present disclosure include gastric cancer cells (e.g., HGC-27 cells); non-small cell lung cancer (NSCLC) cells, colorectal cancer cells (e.g., DLD-1 cells), H23 lung adenocarcinoma cells, Ramos cells, T-cell acute lymphoblastic leukemia (T-ALL) cells, CCRF-CEM cells, acute myeloid leukemia (AML) cells (e.g., HL60 cells), small-cell lung cancer (SCLC) cells (e.g., NCI-H69 cells), human glioblastoma cells (e.g., U118-MG cells), prostate cancer cells (e.g., PC-3 cells), HER-2-overexpressing human breast cancer cells (e.g., SK-BR-3 cells), pancreatic cancer cells (e.g., Mia-PaCa-2)).

In exemplary embodiments, one or more analytes is a virus. In some cases, the virus is directly detected, e.g., a nucleic acid or an antigen specific to the virus is detected. In some cases, the virus is indirectly detected, e.g., detection of anti-virus antibodies produced by a subject can indicate the presence of a virus, or the presence of a virus induces hemagglutination in blood. For example, viruses that may be detected by the assays of the present disclosure include animal, plant, fungal and bacterial viruses. In other embodiments, viruses that may be detected by the assays of the present disclosure include those which impact animals, especially mammals, in particular humans and domestic animals. In still other embodiments, viruses that may be detected by the assays of the present disclosure include, but are not limited to, Papovaviruses, e.g. polyoma virus and SV40; Poxviruses, e.g. vaccinia virus and variola (smallpox); Adenoviruses, e.g., human adenovirus; Herpesviruses, e. g. Human Herpes Simplex types I and II; Parvoviruses, e.g. adeno associated virus (AAV); Reoviruses, e.g., rotavirus and reovirus of humans; Picornaviruses, e.g. poliovirus; Togaviruses, including the alpha viruses (group A), e.g. Sindbis virus and Semliki forest virus (SFV) and the flaviviruses (group B), e.g. dengue virus, yellow fever virus and the St. Louis encephalitis virus; Retroviruses, e. g. lentiviruses, HIV I and II, Rous sarcoma virus (RSV), and mouse leukemia viruses; Rhabdoviruses, e.g. vesicular stomatitis virus (VSV) and rabies virus; Paramyxoviruses, e.g. mumps virus, measles virus and Sendai virus; Arena viruses, e.g., lassa virus; Bunyaviruses, e.g., bunyawere (encephalitis); Coronaviruses, e.g. common cold, GI distress viruses, Orthomyxovirus, e.g., influenza; Caliciviruses, e.g., Norwak virus, Hepatitis E virus; Filoviruses, e.g., ebola virus and Marburg virus; and Astroviruses, e.g. astrovirus, among others. Specific examples of viruses include, but are not limited to, Sin Nombre virus, influenza (especially H5N1 influenza), Herpes Simplex Virus (HSV1 and HSV-2), Coxsackie virus, Human immunodeficiency virus (I and II), Andes virus, Dengue virus, Epstein-Barr virus (mononucleosis), Variola (smallpox) and other pox viruses, West Nile virus, hepatitis viruses (e.g., A, B, C, D, and E), HPV, SARS-COV-2 (COVID-19), CMV, Parvovirus B19, Chlamydia, Gonorrhea, Zika Virus, Chikungunya Virus, Babesia, Malaria, and Usutu virus.

In some embodiments, one or more analytes may be a bacterium. In some cases, the bacterium is directly detected, e.g., a nucleic acid or an antigen specific to the bacterium is detected. In some cases, the bacterium is indirectly detected, e.g., detection of anti-bacteria antibodies produced by a subject can indicate the presence of bacteria, or the presence of bacterial enzyme activity products can indicate the presence of bacteria. For example, bacteria that may be detected by assays of the present disclosure include, but are not limited to, Achromobacter denitrificans, Achromobacter xylosoxidans, Acinetobacter baumannii, Acinetobacter calcoaceticus, Actinomyces israelii, Aerococcus christensenii, Aeromonas hydrophile, Aeromonas sobria, Aggregatibacter actinomycetemcomitans, Alcaligenes faecalis, Alistipes onderdonkii, Anaerococcus vaginalis, Anaeroglobus geminatus, Arcanobacterium haemolyticum, Arcanobacterium pyogenes, Arthrobacter cumminsii, Atopobium vaginae, Bacillus anthracis, Bacillus cereus, Bacillus coagulans, Bacillus licheniformis, Bacillus megaterium, Bacillus pumilus, Bacillus sphaericus, Bacillus subtilis, Bacteroides dorei, Bacteroides finegoldii, Bacteroides fragilis, Bacteroides nordii, Bacteroides salyersiae, Bacteroides thetaiotaomicron, Bacteroides uniformis, Bacteroides vulgatus, Bartonella henselae, Bartonella quintana, Bifidobacterium bifidum, Bifidobacterium breve, Bilophila wadsworthia, Bordetella pertussis, Borrelia burgdorferi, Borrelia recurrentis, Brevibacillus laterosporus, Brucella abortus, Brucella melitensis, Brucella suis, Burkholderia cepacia, Burkholderia mallei, Burkholderia pseudomallei, Campylobacter coli, Campylobacter curvus, Campylobacter jejuni, Campylobacter rectus, Capnocytophaga gingivalis, Capnocytophaga granulosa, Capnocytophaga haemolytica, Capnocytophaga sputigena, Cardiobacterium hominis, Chryseobacterium meningosepticum, Citrobacter amalonaticus, Citrobacter freundii, Citrobacter koseri, Clostridium butyricum, Clostridium difficile, Clostridium histolyticum, Clostridium hylemonae, Clostridium paraputrificum, Clostridium perfringens, Clostridium septicum, Clostridium sporogenes, Clostridium subterminale, Clostridium tertium, Clostridium tetani, Corynebacterium amycolatum, Corynebacterium confusum, Corynebacterium diphtheriae, Corynebacterium glucuronolyticum, Corynebacterium jeikeium, Corynebacterium kroppenstedtii, Corynebacterium macginleyi, Corynebacterium minutissimum, Corynebacterium pseudodiphtheriticum, Corynebacterium pseudotuberculosis, Corynebacterium riegelii, Corynebacterium tuberculostearicum, Corynebacterium ulcerans, Corynebacterium xerosis, Edwardsiella tarda, Eggerthella lenta, Eikenella corrodens, Elizabethkingia meningoseptica, Empedobacter brevis, Enterobacter aerogenes, Enterobacter aerogenes, Enterobacter cloacae, Enterobacter sakazakii, Enterococcus avium, Enterococcus bovis, Enterococcus casseliflavus, Enterococcus cecorum, Enterococcus dispar, Enterococcus durans, Enterococcus faecium, Enterococcus flavescens, Enterococcus gallinarum, Enterococcus gilvus, Enterococcus hirae, Enterococcus italicus, Enterococcus malodoratus, Enterococcus mundtii, Enterococcus pallens, Enterococcus pseudoavium, Enterococcus raffinosus, Enterococcus sanguinicola, Erysipelothrix rhusiopathiae, Escherichia albertii, Escherichia coli, Eubacterium lentum, Eubacterium limosum, Finegoldia magna, Francisella tularensis, Fusobacterium necrophorum, Fusobacterium nucleatum, Fusobacterium periodonticum, Fusobacterium varium, Gardnerella vaginalis, Gemella morbillorum, Geobacillus stearothermophilus, Granulicatella adiacens, Haemophilus ducreyi, Haemophilus influenzae, Haemophilus parainfluenzae, Hafnia alvei, Halomonas venusta, Helicobacter cinaedi, Helicobacter pylori, Kingella kingae, Klebsiella granulomatis, Klebsiella oxytoca, Klebsiella pneumoniae, Lactobacillus acidophilus, Lactobacillus crispatus, Lactobacillus delbrueckii, Lactobacillus jensenii, Lactococcus garvieae, Legionella pneumophila, Leptospira interrogans, Listeria monocytogenes, Micrococcus luteus, Moraxella catarrhalis, Morganella morganii, Mycoplasma genitalium, Mycoplasma hominis, Neisseria gonorrhoeae, Neisseria meningitidis, Nocardia cyriacigeorgica, Odoribacter splanchnicus, Pantoea agglomerans, Parabacteroides distasonis, Parvimonas micra, Pasteurella multocida, Pediococcus damnosus, Peptoniphilus asaccharolyticus, Peptoniphilus gorbachii, Peptostreptococcus anaerobius, Plesiomonas shigelloides, Porphyromonas asaccharolytica, Porphyromonas gingivalis, Prevotella bivia, Prevotella bivia, Prevotella corporis, Prevotella intermedia, Prevotella melaninogenica, Prevotella nigrescens, Prevotella timonensis, Prevotella veroralis, Propionibacterium acnes, Propionibacterium avidum, Propionibacterium granulosum, Proteus mirabilis, Proteus vulgaris, Providencia rettgeri, Providencia stuartii, Pseudomonas aeruginosa, Pseudomonas fluorescens, Pseudomonas putida, Rothia dentocariosa, Rothia mucilaginosa, Salmonella enterica, Serratia marcescens, Serratia plymuthica, Shigella boydii, Shigella dysenteriae, Shigella flexneri, Shigella sonnei, Spirillum minus, Staphylococcus aureus, Staphylococcus auricularis, Staphylococcus capitis, Staphylococcus caprae, Staphylococcus carnosus, Staphylococcus cohnii, Staphylococcus epidermidis, Staphylococcus haemolyticus, Staphylococcus hominis, Staphylococcus lugdunensis, Staphylococcus pasteuri, Staphylococcus pettenkoferi, Staphylococcus pulvereri, Staphylococcus saccharolyticus, Staphylococcus saprophyticus, Staphylococcus schleiferi, Staphylococcus simulans, Staphylococcus warneri, Staphylococcus xylosus, Stenotrophomonas maltophilia, Streptobacillus moniliformis, Streptococcus agalactiae, Streptococcus anginosus, Streptococcus bovis, Streptococcus canis, Streptococcus constellatus, Streptococcus dysgalactiae, Streptococcus equi, Streptococcus gallolyticus, Streptococcus gordonii, Streptococcus infantarius, Streptococcus iniae, Streptococcus intermedius, Streptococcus lutetiensis, Streptococcus mitis, Streptococcus mutans, Streptococcus oralis, Streptococcus pasteurianus, Streptococcus pneumoniae, Streptococcus porcinus, Streptococcus pyogenes, Streptococcus salivarius, Streptococcus salivarius, Streptococcus sanguinis, Streptococcus sobrinus, Streptococcus suis, Streptococcus vestibularis, Sutterella wadsworthensis, Treponema pallidum, Ureaplasma parvum, Vagococcus fluvialis, Veillonella atypica, Veillonella parvula, Vibrio alginolyticus, Vibrio cholerae, Vibrio fluvialis, Vibrio parahaemolyticus, Vibrio vulnificus, Yersinia enterocolitica, Yersinia pestis, Yersinia pseudotuberculosis.

In exemplary embodiments, one or more analytes may be a fungus. In some cases, the fungus is directly detected, e.g., a nucleic acid or an antigen specific to the fungus is detected. In some cases, the fungus is indirectly detected, e.g., a cell wall component of a fungus released into the blood can indicate the presence of the fungus. In certain embodiments, fungi that may be detected by assays of the present disclosure include, but are not limited to, fungi from a fungal genera selected from the group consisting of Candida, Aspergillus, Rhyzopus, Cryptococcus, Histoplasma, Pneumocystis, Stachybotrys, Sporothrix, Trichophyton, Microsporum, Blastomyces, Mucoromycotina, Coccidioides, Exserohilum, Cladosporium, Coccoides, Encephalitozoon, Encephalitozoon, Fusarium, Lichtheimia, Mortierella, Malassezia, Prototheca, Pythium, Rhodotorula, Fusarium, Thielaviopsis, Verticillium, Magnaporthe, Sclerotinia, Ustilago, Rhizoctonia, Puccinia, Armillaria, Botrytis, Blumeria, Mycosphaerella, Colletotrichum, Melampsora, Saprolegniasis, Ichthyosporidium, Exophiala, Branchiomycosis, and Penicillium. Specific examples of fungal species that can be detected by the assays of the present disclosure include, but are not limited to, Candida albicans, C. glabrata, C. parapsilosis, C. tropicalis, and C. auris; Cryptococcus neoformans and C. gattii; Coccidioides immitis and C. posadasii; Histoplasma capsulatum; Blastomyces dermatitidis; and Pneumocystis jirovecii.

In some embodiments, one or more analytes may be a protozoa. In some cases, the protozoan is directly detected, e.g., a nucleic acid or an antigen specific to the protozoan is detected. In other cases, the protozoan is indirectly detected, e.g., a metabolic product of the protozoan can indicate the presence of the protozoan. In some cases, classes of protozoa that may be detected by assays of the present disclosure include, but are not limited to, Plasmodium (malaria), Leishmania (leishmaniasis), Trypanosoma (sleeping sickness and Chagas disease), Cryptosporidium, Giardia, Toxoplasma, Babesia, Balantidium and Entamoeba. Specific examples of protozoa that can be detected by the assays of the present disclosure include, but are not limited to, Plasmodium falciparum, Plasmodium ovale, Plasmodium malariae, Plasmodium vivax, Leishmania donovani, Trypanosoma brucei, Trypanosoma cruzi, Toxoplasma gondii and Babesia microti.

In exemplary embodiments, one or more analytes may be a prion protein. In some cases, the prion is directly detected, e.g., a nucleic acid or an antigen specific to the prion is detected. In other cases, the presence of prions or potential for prion formation is detected by identifying a mutation in a nucleic acid sequence. In some cases, the presence of structures formed by prions can indicate the presence of prions. In some cases, the prion is indirectly detected, e.g., biochemical changes induced by prion formation can indicate the presence of prions. In some cases, prions are amplified prior to detection using methods such as protein misfolding cyclic amplification (PMCA) or real-time quaking-induced conversion (RT-QUIC). Exemplary prion proteins include, but are not limited to, Scrapie (Sheep and goats), transmissible mink encephalopathy (TME), chronic wasting disease (CWD) in mule deer and elk, bovine spongiform encephalopathy (BSE) cattle, feline spongiform encephalopathy (FSE) in cats, exotic ungulate encephalopathy (EUE), Kuru in humans, Creutzfeldt-Jakob disease (CJD) in humans, Fatal familial insomnia (FFI) in humans and Gerstmann-Strässler-Scheinker syndrome (GSS) in humans.

In some embodiments, one or more analytes measured by the assays of the devices and methods of the present disclosure may be a blood component. Examples of blood components that may be detected by assays of the present disclosure include, but are not limited to, red blood cells, hemoglobin, white blood cells (including neutrophils, lymphocytes, monocytes, eosinophils, and basophils), platelets, reticulocytes, and nucleated red blood cells. Various measurements of different blood components may be performed, including, but not limited to, cell count, cell size, cell complexity, granularity, hematocrit, mean corpuscular volume, mean corpuscular hemoglobin, and mean corpuscular hemoglobin concentration.

In some embodiments, one or more analytes may be a biomolecule. Non-limiting examples of biomolecules include macromolecules such as, for example, proteins, lipids, and carbohydrates. In certain instances, the analyte may be hormones, antibodies, growth factors, cytokines, electrolytes (e.g., sodium, potassium, and chloride), enzymes (e.g., alanine aminotransferase, aspartate aminotransferase, lactate dehydrogenase, and amylase), receptors (e.g., neural, hormonal, nutrient, and cell surface receptors) or their ligands, cancer markers (e.g., PSA, TNF-alpha), markers of myocardial infarction (e.g., troponin, creatine kinase, B-type natriuretic peptide (also known as brain natriuretic peptide; BNP), N-terminal prohormone of brain natriuretic peptide (NT-proBNP) and the like), toxins, drugs (e.g., therapeutic drugs, drugs of addiction), metabolic agents (e.g., including vitamins and minerals), metabolic products (e.g., glucose, urea nitrogen triglycerides, uric acid), nutrients, and the like. Non-limiting embodiments of protein analytes include peptides, polypeptides, protein fragments, protein complexes, fusion proteins, recombinant proteins, phosphoproteins, glycoproteins, lipoproteins, and the like. In certain embodiments, the analyte may be a post-translationally modified protein (e.g., phosphorylated, methylated, glycosylated protein). In certain embodiments, the analyte is a nucleic acid. In certain embodiments, the analyte is a protein or a small molecule.

A non-limiting list of analytes that may be analyzed by the devices presented herein include Aβ42 amyloid beta-protein, fetuin-A, tau, secretogranin II, prion protein, Alpha-synuclein, tau protein, neurofilament light chain, parkin, PTEN induced putative kinase 1, DJ-1, leucine-rich repeat kinase 2, mutated ATP13A2, Apo H, ceruloplasmin, Peroxisome proliferator-activated receptor gamma coactivator-1 alpha (PGC-1α), transthyretin, Vitamin D-binding Protein, proapoptotic kinase R (PKR) and its phosphorylated PKR (pPKR), CXCL13, IL-12p40, CXCL13, IL-8, Dkk-3 (semen), p14 endocan fragment, Serum, ACE2, autoantibody to CD25, hTERT, CAI25 (MUC 16), VEGF, sIL-2, Osteopontin, Human epididymis protein 4 (HE4), Alpha-Fetoprotein, Albumin, albuminuria, microalbuminuria, neutrophil gelatinase-associated lipocalin (NGAL), interleukin 18 (IL-18), Kidney Injury Molecule-1 (KIM-1), Liver Fatty Acid Binding Protein (L-FABP), LMP1, BARF1, IL-8, carcinoembryonic antigen (CEA), BRAF, CCNI, EGRF, FGF19, FRS2, GREB1, and LZTS1, alpha-amylase, carcinoembryonic antigen, CA 125, IL8, thioredoxin, beta-2 microglobulin levels-monitor activity of the virus, tumor necrosis factor-alpha receptors-monitor activity of the virus, CA15-3, follicle-stimulating hormone (FSH), leutinizing hormone (LH), T-cell lymphoma invasion and metastasis 1 (TIAM1), N-cadherin, EC39, amphiregulin, dUTPase, secretory gelsolin (pGSN), PSA (prostate specific antigen), thymosin β15, insulin, plasma C-peptide, glycosylated hemoglobin (HBA1c), C-Reactive Protein (CRP), Interleukin-6 (IL-6), ARHGDIB (Rho GDP-dissociation inhibitor 2), CFL1 (Cofilin-1), PFN1 (profilin-1), GSTP1 (Glutathione S-transferase P), S100A11 (Protein S100-A11), PRDX6 (Peroxiredoxin-6), HSPE1 (10 kDa heat shock protein, mitochondrial), LYZ (Lysozyme C precursor), GPI (Glucose-6-phosphate isomerase), HIST2H2AA (Histone H2A type 2-A), GAPDH (Glyceraldehyde-3-phosphate dehydrogenase), HSPG2 (Basement membrane-specific heparan sulfate proteoglycan core protein precursor), LGALS3BP (Galectin-3-binding protein precursor), CTSD (Cathepsin D precursor), APOE (Apolipoprotein E precursor), IQGAP1 (Ras GTPase-activating-like protein IQGAP1), CP (Ceruloplasmin precursor), and IGLC2 (IGLC1 protein), PCDGF/GP88, EGFR, HER2, MUC4, IGF-IR, p27 (kip1), Akt, HER3, HER4, PTEN, PIK3CA, SHIP, Grb2, Gab2, PDK-1 (3-phosphoinositide dependent protein kinase-1), TSC1, TSC2, mTOR, MIG-6 (ERBB receptor feedback inhibitor 1), S6K, src, KRAS, MEK mitogen-activated protein kinase 1, cMYC, TOPO II topoisomerase (DNA) II alpha 170 kDa, FRAP1, NRG1, ESR1, ESR2, PGR, CDKNIB, MAP2K1, NEDD4-1, FOXO3A, PPP1R1B, PXN, ELA2, CTNNB1, AR, EPHB2, KLF6, ANXA7, NKX3-1, PITX2, MKI67, PHLPP, adiponectin (ADIPOQ), fibrinogen alpha chain (FGA), leptin (LEP), advanced glycosylation end product-specific receptor (AGER aka RAGE), alpha-2-HS-glycoprotein (AHSG), angiogenin (ANG), CD14 molecule (CD14), ferritin (FTH1), insulin-like growth factor binding protein 1 (IGFBP1), interleukin 2 receptor, alpha (IL2RA), vascular cell adhesion molecule 1 (VCAM1) and Von Willebrand factor (VWF), myeloperoxidase (MPO), ILla, TNFα, perinuclear anti-neutrophil cytoplasmic antibody (p-ANCA), lactoferrin, calprotectin, Wilm's Tumor-1 protein, Aquaporin-1, MLL3, AMBP, VDAC1, E. coli enterotoxins (heat-labile exotoxin, heat-stable enterotoxin), influenza HA antigen, tetanus toxin, diphtheria toxin, botulinum toxins, Shiga toxin, Shiga-like toxin I, Shiga-like toxin II, Clostridium difficile toxins A and B, etc.

Exemplary targets of nucleic acid aptamers that may be measured in a sample such as an environmental sample, a biological sample obtained from a patient or subject in need using the subject devices include: drugs of abuse (e.g. cocaine), protein biomarkers (including, but not limited to, Nucleolin, nuclear factor-kB essential modulator (NEMO), CD-30, protein tyrosine kinase 7 (PTK7), vascular endothelial growth factor (VEGF), MUC1 glycoform, immunoglobulin μ Heavy Chains (IGHM), Immunoglobulin E, αvβ3 integrin, α-thrombin, HIV gp120, NF-κB, E2F transcription factor, HER3, Plasminogen activator inhibitor, Tenascin C, CXCL12/SDF-1, prostate specific membrane antigen (PSMA), gastric cancer cells, HGC-27); cells (including, but not limited to, non-small cell lung cancer (NSCLC), colorectal cancer cells, (DLD-1), H23 lung adenocarcinoma cells, Ramos cells, T-cell acute lymphoblastic leukemia (T-ALL) cells, CCRF-CEM, acute myeloid leukemia (AML) cells (HL60), small-cell lung cancer (SCLC) cells, NCIH69, human glioblastoma cells, U118-MG, PC-3 cells, HER-2-overexpressing human breast cancer cells, SK-BR-3, pancreatic cancer cell line (Mia-PaCa-2)); and infectious agents (including, but not limited to, Mycobacterium tuberculosis, Staphylococcus aureus, Shigella dysenteriae, Escherichia coli O157:H7, Campylobacter jejuni, Listeria monocytogenes, Pseudomonas aeruginosa, Salmonella O8, Salmonella enteritidis).

Exemplary targets of protein or peptide aptamers that may be measured in a sample obtained from a patient or subject in need using the subject devices include, but are not limited to: HBV core capsid protein, CDK2, E2F transcription factor, Thymidylate synthase, Ras, EB1, and Receptor for Advanced Glycated End products (RAGE). Aptamers, and use and methods of production thereof are reviewed in e.g., Shum et al., J Cancer Ther. 2013 4:872; Zhang et al., Curr Med Chem. 2011; 18:4185; Zhu et al., Chem Commun (Camb). 2012 48:10472; Crawford et al., Brief Funct Genomic Proteomic. 2003 2:72; Reverdatto et al., PLOS One. 2013 8: e65180.

In certain cases, a biological sample (e.g., human blood sample) that contains or is suspected of containing a target nucleic acid may undergo preparation/processing prior to detection by a primary analysis unit of a system of the present disclosure. In some embodiments, the preparation/processing may include the following steps: i) isolation of total nucleic acid that contains a target nucleic acid from the sample, ii) optionally, enrichment of the target nucleic acid, iii) amplification of the target nucleic acid, and iv) processing of the amplified target nucleic acid. Each step can be performed manually, automatically, or by a combination thereof.

In certain embodiments, the analyte is not amplified (i.e., the copy number of the analyte is not increased) prior to the measurement of the analyte. For example, in cases where the analyte is DNA or RNA, the analyte is not replicated to increase copy numbers of the analyte.

### III. Types of Assays and Analysis

### Digital (Individual) Analysis

The devices and methods disclosed herein are capable of performing digital analysis of the analytes of the present disclosure. The placement of single microparticles bound to the analyte molecules into wells allows for a digital readout. For example, for a low number of positive wells (<˜70% positive) Poisson statistics can be used to quantitate the analyte concentration in a digital format. A digital signal may be used for lower analyte concentrations. As used herein, a “positive well” refers to a well that has a signal related to presence of a microparticle bound to the analyte molecule, which signal is above a threshold value. As used herein, a “negative well” refers to a well that may not have a signal related to presence of a microparticle bound to the analyte molecule. In certain embodiments, the signal from a negative well may be at a background level, i.e., below a threshold value.

Because every single analyte, as an end-point entity, can be detected in the context of digital detection, the components and methods associated with digital detection can significantly increase detection sensitivity for sample analysis compared to systems using analog optical detection. As such, digital detection can be performed using a lower concentration of analyte which can allow for decreased time to process the sample for detection. Additionally, or alternatively, detection can be performed using a smaller sample volume, less reagent material, less conjugate material, fewer microparticles, or any combination of these, which can reduce costs to perform each assay. As such, and as described herein, sample preparation time can be improved due at least in part to less sample manipulation involved (e.g., faster washing times) and/or improved kinetics of reactions achieved using a lower sample volume, less reagent or conjugate material, and/or fewer microparticles to obtain an analyte concentration suitable for detection. Assays using less sample volume and/or reagent material can be performed using smaller equipment, which can reduce the footprint of the laboratory system for performing the assays as discussed further herein. In addition, or as a further alternative, increased detection sensitivity can provide additional benefits when used with multiplexing

Digital detection can provide increased sensitivity due at least in part to a reduction of noise during detection relative to the signal being measured, for example, producing a higher signal-to-noise ratio. Such improved signal-to-noise ratios are possible by coupling the analyte, e.g., a particular nucleic acid, protein, or nucleic acid associated with a protein or microparticles, to an independently detectable end-point entity. For example, but not limitation, amplified nucleic acids or proteins can be immobilized to microparticles and labeled with detectable conjugates, where the conjugate is a detectable end-point entity in that it can emit an independently detectable signal, either directly or via the conversion of a substrate.

Additionally, or alternatively, and in accordance with another aspect of the disclosed subject matter, the detection operation employs a digital microwell detection process. Additionally, or alternatively, a support medium, such as, but not limited to, microparticles or other labels, can be mixed with the sample in order to perform the digital detection process after amplification. In certain embodiments, reagents including antibodies and coated microparticles can be combined.

### Immunoassays

The target analyte, and/or peptides of fragments thereof, may be analyzed using anti-analyte antibodies in an immunoassay. The presence or amount can be determined using antibodies and detecting specific binding to the analyte. For example, the antibody, or antibody fragment thereof, may specifically bind to the target analyte.

The presence or amount of analyte present in a sample may be readily determined using an immunoassay, such as sandwich immunoassay (e.g., monoclonal-monoclonal sandwich immunoassays, monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, MN)). An example of a point-of-care device that can be used is i-STAT® (Abbott, Laboratories, Abbott Park, IL). Other methods that can be used include a chemiluminescent microparticle immunoassay, in particular one employing the ARCHITECT® automated analyzer (Abbott Laboratories, Abbott Park, IL), as an example. Other methods include, for example, mass spectrometry, and immunohistochemistry (e.g., with sections from tissue biopsies), using anti-analyte antibodies (monoclonal, polyclonal, chimeric, humanized, human, etc.) or antibody fragments thereof against analyte. Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to the analyte can be detected via direct labels, such as fluorescent or luminescent tags, metals and radionuclides attached to the antibody or via indirect labels, such as alkaline phosphatase or horseradish peroxidase.

The use of immobilized antibodies or antibody fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like.

A homogeneous format may be used. For example, after the sample is obtained from a subject, a mixture is prepared. The mixture contains the sample being assessed for analyte, a first specific binding partner, and a second specific binding partner. The order in which the sample, the first specific binding partner, and the second specific binding partner are added to form the mixture is not critical. The sample is simultaneously contacted with the first specific binding partner and the second specific binding partner. In some embodiments, the first specific binding partner and any analyte contained in the sample may form a first specific binding partner-analyte-antigen complex and the second specific binding partner may form a first specific binding partner-analyte of interest-second specific binding partner complex. In some embodiments, the second specific binding partner and any analyte contained in the sample may form a second specific binding partner-analyte-antigen complex and the first specific binding partner may form a first specific binding partner-analyte of interest-second specific binding partner complex.

A heterogeneous format may be used. For example, after the sample is obtained from a subject, a first mixture is prepared. The mixture contains the sample being assessed for analyte and a first specific binding partner, wherein the first specific binding partner and any analyte contained in the sample form a first specific binding partner-analyte-antigen complex. The order in which the sample and the first specific binding partner are added to form the mixture is not critical.

The first specific binding partner may be immobilized on a solid phase. The solid phase used in the immunoassay (for the first specific binding partner and, optionally, the second specific binding partner) can be any solid phase known in the art, such as, but not limited to, microparticle, a bead, a test tube, a microtiter plate, a cuvette, a membrane, a scaffolding molecule, a film, a filter paper, a disc, and a chip. Microparticle may be ferromagnetic, ferrimagnetic, paramagnetic, superparamagnetic or ferrofluidic. Exemplary ferromagnetic materials include Fe, Co, Ni, Gd, Dy, CrO2, MnAs, MnBi, EuO, and NiO/Fe. Examples of ferrimagnetic materials include NiFe2O4, CoFe2O4, Fe3O4 (or FeO·Fe2O3). Microparticles can have a solid core portion that is susceptible to a magnetic field and is surrounded by one or more non-magnetic layers. Alternately, the portion susceptible to a magnetic field can be a layer around a non-magnetic core. The solid support on which the first specific binding member is immobilized may be stored in dry form or in a liquid. The microparticles may be subjected to a magnetic field prior to or after contacting with the sample with a microparticle on which the first specific binding member is immobilized.

After the mixture containing the first specific binding partner-analyte antigen complex is formed, any unbound analyte is removed from the complex using any technique known in the art. For example, the unbound analyte can be removed by washing. Desirably, however, the first specific binding partner is present in excess of any analyte present in the sample, such that all analyte that is present in the sample is bound by the first specific binding partner.

After any unbound analyte is removed, a second specific binding partner is added to the mixture to form a first specific binding partner-analyte of interest-second specific binding partner complex.

### Sandwich Assay

A sandwich immunoassay measures the amount of antigen between two layers of antibodies (i.e., at least one capture antibody) and a detection antibody (i.e., at least one detection antibody). The capture antibody and the detection antibody bind to different epitopes on the antigen, e.g., analyte of interest. Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte in a sample. More specifically, the at least two antibodies bind to certain epitopes of analyte forming an immune complex which is referred to as a “sandwich”. One or more antibodies can be used to capture the analyte in the sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. Antibodies are selected so that the one or more first antibodies brought into contact with a sample suspected of containing analyte do not bind to all or part of an epitope recognized by the second or subsequent antibodies, thereby interfering with the ability of the one or more second detection antibodies to bind to the analyte.

The antibodies may be used as a first antibody in said immunoassay. The antibody immunospecifically binds to epitopes on analyte. In addition to the antibodies of the present disclosure, said immunoassay may comprise a second antibody that immunospecifically binds to epitopes that are not recognized or bound by the first antibody.

A sample suspected of containing analyte can be contacted with at least one first capture antibody (or antibodies) and at least one second detection antibodies either simultaneously or sequentially. In the sandwich assay format, a sample suspected of containing analyte is first brought into contact with the at least one first capture antibody that specifically binds to a particular epitope under conditions which allow the formation of a first antibody-analyte antigen complex. If more than one capture antibody is used, a first multiple capture antibody-analyte antigen complex is formed. In a sandwich assay, the antibodies, preferably, the at least one capture antibody, are used in molar excess amounts of the maximum amount of analyte expected in the sample. For example, from about 5 μg/mL to about 1 mg/mL of antibody per ml of microparticle coating buffer may be used.

Provided herein are methods for measuring or detecting a target analyte present in a biological sample. In some embodiments, the target analyte is a protein. In such methods, a sample comprising a target analyte is deposited into a primary zone. In some embodiments, the primary zone contain microparticles or microparticles and assisting particles. Optionally, a lysis buffer may be added to the sample deposited in the primary. Following sample deposit, microparticles attached to a first specific binding partner (e.g., a capture antibody) that specifically binds to the target analyte present in the sample are added to the sample. In some embodiments, microparticles attached to a first specific binding partner (e.g., a capture antibody) that specifically binds to the target analyte present in the sample and assisting particles are added to the sample The sample may be incubated with the microparticles or microparticles and assisting microparticles for a time sufficient to allow binding of the first specific binding partner to an analyte present in the sample thereby producing microparticles bound to the target analyte where the microparticles are attached to the first specific binding partner and the first specific binding partner is bound to the target analyte.

Optionally, the sample comprising the target analyte may be mixed with the microparticles attached to the first specific binding partner or microparticles attached to the first specific binding partner and assisting particles. Mixing may be achieved using any of the mixing methods described above. Optionally, the microparticles bound to the target analyte or microparticles bound to the target analyte and assisting particles may be subjected to a magnetic field to move the microparticles bound to the target analyte to one or more primary zones containing wash buffer. Next, the microparticles bound to the target analyte or microparticles bound to the target analyte and assisting particles may be subjected to a magnetic field to move the microparticles bound to the target analyte or microparticles bound to the target analyte and assisting particles to a primary zone containing a second specific binding partner (e.g., a detection antibody). In some embodiments, the second specific binding partner is referred to as a conjugate. The second specific binding partner may be detectably labeled. The label may be any label that can be optically detected. For example, the label may be a fluorescent label or a label that reacts with a substrate to produce a detectable label. The microparticles bound to the target analyte may be incubated for a period of time sufficient for the second specific binding partner to bind the analyte bound to the first binding member thereby producing microparticles bound to detectably labeled target analyte. Optionally, the microparticles bound to the detectably labeled target analyte or microparticles bound to the detectably labeled target analyte and assisting particles may be subjected to a magnetic field to move the microparticles to one or more primary zones containing wash buffer.

Next, the microparticles bound to the detectably labeled target analyte or microparticles bound to the detectably labeled target analyte and assisting particles may be subjected to a magnetic field to move the microparticles to the detection zone in a sample analysis region. As explained herein, the immunoassay may be carried out in the sample processing region. The microparticles bound to the detectably labeled target analyte may be allowed to settle into the array of wells in the detection zone. The microparticles may settle using gravitational force or by applying electric or magnetic force. Optionally, a hydrophilic liquid may be added to the detection zone that reacts with the second specific binding partner to produce a detectable signal. In some embodiments, the hydrophilic liquid is a substrate solution. Next, the wells containing the microparticles bound to the detectably labeled target analyte are sealed using a hydrophobic liquid (e.g., oil). Lastly, the wells are analyzed thereby detecting the detectable signal produced by the hydrophilic liquid reacting with the second specific binding partner. The analysis may be any analysis that is capable of detecting the detectable signal produced by the substrate reacting with the second specific binding partner. In some embodiments, the analysis is imaging the wells.

### Anti-Analyte Capture Antibody

Optionally, prior to contacting the sample with the at least one first capture antibody, the at least one first capture antibody can be bound to a solid support (e.g., a microparticle) which facilitates the separation the first antibody-analyte complex from the sample. Any solid support known in the art can be used, including but not limited to, solid supports made out of polymeric materials in the forms of wells, tubes, or beads (such as a microparticle). The antibody (or antibodies) can be bound to the solid support by adsorption, by covalent bonding using a chemical coupling agent or by other means known in the art, provided that such binding does not interfere with the ability of the antibody to bind analyte. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl) carbodiimide.

After the sample suspected of containing analyte is incubated in order to allow for the formation of a first capture antibody (or multiple antibody)-analyte complex. The incubation can be carried out at a pH of from about 4.5 to about 10.0, at a temperature of from about 2° C. to about 45° C., and for a period from at least about one (1) minute to about eighteen (18) minutes, from about 2-6 minutes, from about 7-12 minutes, from about 5-15 minutes, or from about 3-4 minutes.

### Detection Antibody

After formation of the first/multiple capture antibody-analyte complex, the complex is then contacted with at least one second detection antibody (under conditions that allow for the formation of a first/multiple antibody-analyte antigen-second antibody complex). In some embodiments, the sample is contacted with the detection antibody simultaneously with the capture antibody. If the first antibody-analyte complex is contacted with more than one detection antibody, then a first/multiple capture antibody-analyte-multiple antibody detection complex is formed. As with first antibody, when the at least second (and subsequent) antibody is brought into contact with the first antibody-analyte complex, a period of incubation under conditions similar to those described above is required for the formation of the first/multiple antibody-analyte-second/multiple antibody complex. Preferably, at least one second antibody contains a detectable label. The detectable label can be bound to the at least one second antibody prior to, simultaneously with or after the formation of the first/multiple antibody-analyte-second/multiple antibody complex. Any detectable label known in the art can be used.

Chemiluminescent assays can be performed in accordance with the methods described in Adamczyk et al., Anal. Chim. Acta 579 (1): 61-67 (2006). Desirably, the formation of pseudobases in neutral or basic solutions employing an acridinium aryl ester is avoided, such as by acidification. The chemiluminescent response is then recorded. In this regard, the time for recording the chemiluminescent response will depend, in part, on the delay between the addition of the reagents and the particular acridinium employed.

The order in which the sample and the specific binding partner(s) are added to form the mixture for chemiluminescent assay is not critical. If the first specific binding partner is detectably labeled with an acridinium compound, detectably labeled first specific binding partner-antigen complexes form. Alternatively, if a second specific binding partner is used and the second specific binding partner is detectably labeled with an acridinium compound, detectably labeled first specific binding partner-analyte-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample. Other labels other than chemiluminescent labels can be employed. For instance, enzymatic labels (including but not limited to alkaline phosphatase) can be employed.

The chemiluminescent signal, or other signal, that is generated can be detected using routine techniques known to those skilled in the art. Based on the intensity of the signal generated, the amount of analyte of interest in the sample can be quantified. Specifically, the amount of analyte in the sample is proportional to the intensity of the signal generated. The amount of analyte present can be quantified by comparing the amount of light generated to a standard curve for analyte or by comparison to a reference standard. The standard curve can be generated using serial dilutions or solutions of known concentrations of analyte by mass spectroscopy, gravimetric methods, and other techniques known in the art.

### Forward Competitive Inhibition

In a forward competitive format, an aliquot of labeled analyte of interest (e.g., target analyte) having a fluorescent label, a tag attached with a cleavable linker, etc.) of a known amount is used to compete with analyte of interest in a sample for binding to analyte of interest antibody.

In a forward competition assay, an immobilized specific binding partner (such as an antibody) can either be sequentially or simultaneously contacted with the sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

The labeled analyte of interest, the sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, etc.) while the other antibody-analyte of interest complex does not contain a detectable label. The antibody-analyte of interest complex can be, but does not have to be, separated from the remainder of the sample prior to quantification of the detectable label. Regardless of whether the antibody-analyte of interest complex is separated from the remainder of the sample, the amount of detectable label in the antibody-analyte of interest complex is then quantified. The amount of analyte of interest (such as membrane-associated analyte of interest, soluble analyte of interest, fragments of soluble analyte of interest, variants of analyte of interest (membrane-associated or soluble analyte of interest) or any combinations thereof) in the sample can then be determined, e.g., as described above.

### Reverse Competition Assay

In a reverse competition assay, an immobilized analyte of interest can either be sequentially or simultaneously contacted with a sample and at least one labeled antibody.

The analyte of interest can be bound to a solid support, such as the solid supports discussed above in connection with the sandwich assay format.

The immobilized analyte of interest, sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The amount of analyte of interest in the sample can then be determined by comparing the quantity of detectable label as described above.

### One-Step Immunoassay or “Capture on the Fly”

In a capture on the fly immunoassay, a solid substrate is pre-coated with an immobilization agent. The capture agent, the analyte and the detection agent are added to the solid substrate together, followed by a wash step prior to detection. The capture agent can bind the analyte and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody.

In certain other embodiments, in a one-step immunoassay or “capture on the fly”, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest. The second specific binding member comprises a detectable label and binds to an analyte of interest. The solid support and the first and second specific binding members may be added to a sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected, or their amounts, levels or amounts, measured, determined or assessed.

The use of a capture on the fly assay can be done in a variety of formats as described herein, and known in the art. For example, the format can be a sandwich assay such as described above, but alternately can be a competition assay, can employ a single specific binding member, or use other variations such as are known.

### Controls

It may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, i.e., fluorescent signal intensity, if a fluorescent label is used. Using samples taken from multiple donors, standard curves can be provided for reference levels of the analyte in normal healthy tissue, as well as for “at-risk” levels of the analyte in tissue taken from donors, who may have one or more of the characteristics set forth above.

Thus, in view of the above, a method for determining the presence, amount, or amount of analyte in a sample is provided. The method comprises assaying the sample for analyte by an immunoassay, for example, employing at least one capture antibody that binds to an epitope on analyte and at least one detection antibody that binds to an epitope on analyte which is different from the epitope for the capture antibody and optionally includes a detectable label, and comprising comparing a signal generated by the detectable label as a direct or indirect indication of the presence, amount or concentration of analyte in the sample to a signal generated as a direct or indirect indication of the presence, amount or concentration of analyte in a calibrator. The calibrator is optionally, and is preferably, part of a series of calibrators in which each of the calibrators differs from the other calibrators in the series by the concentration of the analyte.

### Nucleic Acid Analyses

Various amplification methods and components will be known to one of ordinary skill in the art and any convenient method can be used in the devices and methods disclosed herein (see, e.g., Zanoli and Spoto, Biosensors (Basel). 2013 March; 3 (1): 18-43; Gill and Ghaemi, Nucleosides, Nucleotides, and Nucleic Acids, 2008, 27:224-243; Craw and Balachandrana, Lab Chip, 2012, 12, 2469-2486; which are herein incorporated by reference in their entirety). Nucleic acid amplification can comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

Typically, nucleic acid amplification is employed to increase the number of a target nucleic acid (i.e., a target analyte) in the sample, e.g., to thereby facilitate detection of the target nucleic acid. As embodied herein, the nucleic acid amplification methods and system components can be configured to amplify a target nucleic acid using any of a variety or combination of suitable amplification techniques.

As further disclosed herein, after the sample is prepared, e.g., nucleic acids are isolated from the sample, the isolated nucleic acids can be amplified. The devices and methods include contacting the isolated nucleic acids with the amplification oligonucleotides, e.g., forward and reverse primer oligonucleotides, and probes as described herein to form a reaction mixture. The reaction mixture is then placed under amplification conditions. The term “amplification conditions,” as used herein, refers to conditions that promote annealing and/or extension of the amplification oligonucleotides. In certain embodiments, such conditions include contacting the isolated nucleic acids with an “E-Mix” or a “Core Mix”. Typically, an E-Mix is a solution comprising ATP, Phosphocreatine, and buffer. In contrast, a Core Mix typically comprises a collection of proteins necessary to amplify a nucleic acid target. In certain embodiments, such conditions include contacting the isolated nucleic acids with a “MasterMix.” As used herein, a MasterMix refers to a solution comprising all of the components, e.g., nucleotide triphosphates, polymerases, primers, and probes, necessary to amplify a target nucleic acid for subsequent detection, except an activator, which can be separately provided to initiate amplification. For example, an activator is initially dispensed or merged with the sample, the nucleic acid may be isolated with microparticles and the Master Mix is dispensed or merged with the sample. Amplification conditions are well-known in the art and depend on the amplification method selected. In accordance with the disclosed subject matter, amplification conditions encompass a wide range of reaction conditions including, but not limited to, temperature and/or temperature cycling, buffer, salt, ionic strength, pH, and the like.

Additionally, or alternatively, and in accordance with another aspect of the disclosed subject matter, the devices, and methods of the present disclosure include the use of rapid amplification strategies having a duration of about 1 minute to about 60 minutes, about 5 minutes to about 60 minutes, about 8 minutes to about 60 minutes, or in about 8 minutes to about 50 minutes, or in about 8 minutes to about 40 minutes, or in about 8 minutes to about 35 minutes, or in about 8 minutes to about 30 minutes, or in about 8 minutes to about 25 minutes, or about 8 minutes to about 20 minutes, about 1 minute to about 22 minutes, about 5 minutes to about 22 minutes, about 8 minutes to about 22 minutes, about 1 minute to about 20 minutes, about 5 minutes to about 20 minutes, about 8 minutes to about 20 minutes, or about 8 minutes to about 15 minutes from the addition of the reagents sufficient to initiate amplification of a sample of eluted nucleic acids if the targeted nucleic acid(s) is present.

In certain embodiments, the devices, and methods employ isothermal target amplification to amplify target nucleic acid sequences for detection by a device of the present disclosure. Isothermal target amplification methods do not require a thermocycler, and can be easily adapted and integrated into the devices of the present disclosure. As used interchangeably herein, “isothermal amplification reaction”, “isothermal target amplification” and other variations, refers to a target amplification reaction, wherein the temperature does not significantly change during the reaction, i.e., the target amplification reaction is carried out substantially at a single temperature. The temperature of an isothermal amplification reaction does not change over the course of the reaction by more than, e.g., 10° C., 9° C., 8° C., 7° C., 6° C., 5° C., 4° C., 3° C., 2° C., 1° C.

Depending on the method of isothermal amplification of nucleic acids, different enzymes are required for the amplification reaction. Known isothermal methods for amplification of nucleic acids are e.g., helicase-dependent amplification (HDA) (Vincent et al, EMBO reports, 2004. 5 (8): 795-800), thermostable HDA (tHDA) (An et al, J. Biol. Chem, 2005. 280 (32): 28952-28958), strand displacement amplification (SDA) (Walker et al, Nucleic Acids Res, 1992. 20 (7): 1691-6), multiple displacement amplification (MDA) (Dean et al, Proc. Natl. Acad. Sci., 2002. 99 (8): 5261-5266), rolling circle amplification (Liu et al, J. Am. Chem. Soc., 1996 118:1587-1594), single primer isothermal amplification (SPIA) (Dafforn et al, Biotechniques, 2004. 37 (5): 854-7), restriction aided RCA (Wang et al, Genome Res., 2004. 14:2357-2366), transcription mediated amplification (TMA) (Vuorinen et al, J. Clin. Microbiol., 1995. 33:1856-1859), Nucleic Acid Sequence Based Amplification (NASBA) (Kievits et al, J. Virol. Methods, 1991. 35:273-286) and amplification reactions using nicking enzymes, e.g., nicking enzyme amplification reaction (NEAR) (U.S. Patent Application No. US2009017453), amplification reactions using recombination proteins, e.g., recombinase polymerase amplification (RPA) (Piepenburg et al, PLOS Biol., 2004. 4 (7): e204), and Loop-mediated isothermal amplification (LAMP) (Notomi et al, Nucleic Acids Res., 2000. 28 (12): e63) wherein the at least one mesophilic enzyme for amplifying nucleic acids under isothermal conditions is selected from the group consisting of helicase, mesophilic polymerases, mesophilic polymerases having strand displacement activity, nicking enzymes, recombination proteins, ligases, glycosylases and nucleases.

In some embodiments, amplification of a target nucleic acid for subsequent detection on the devices of the present disclosure is achieved by recombinase polymerase amplification (RPA) (see, U.S. Pat. Nos. 7,270,981; 7,485,428 and 8,460,875 herein incorporated by reference). RPA is a single tube isothermal amplification reaction. In some cases, reverse transcriptase can be added to an RPA reaction in order to amplify RNA targets. RPA methods employ three enzymes: a recombinase, a single-stranded DNA binding protein (e.g., E. coli SSB) and a strand-displacing polymerase. Generally: first, a recombinase agent is contacted with a first and a second nucleic acid primer to form a first and a second nucleoprotein primer. Second, the first and second nucleoprotein primers are contacted to a double stranded target nucleic acid sequence to form a first double stranded structure at a first portion of said first strand and form a double stranded structure at a second portion of said second strand so 3′ ends of said first nucleic acid primer and said second nucleic acid primer are oriented towards each other on a given template nucleic acid molecule. Third, 3′ end of said first and second nucleoprotein primers are extended by a strand-displacing polymerase to generate first and second double stranded nucleic acids, and first and second displaced strands of nucleic acid. The second and third steps are repeated until a desired degree of amplification is achieved. A person skilled in the art will be able to recognize and carry out variations on the general RPA method as described above.

A recombinase agent is an enzyme that can coat single-stranded DNA (ssDNA) to form filaments, which can then scan double-stranded DNA (dsDNA) for regions of sequence homology. When homologous sequences are located, the nucleoprotein filament (comprising the recombinase agent) strand invades the dsDNA creating a short hybrid and a displaced strand bubble known as a D-loop. Suitable recombinase agents include the E. coli RecA protein or any homologous protein or protein complex from any phyla. These RecA homologues are generally named Rad51 after the first member of this group to be identified. Other recombinase agents may be utilized in place of RecA, for example, RecT or RecO. Recombinase agents generally require the presence of ATP, ATPYS, or other nucleoside triphosphates and their analogs. Recombinase agents are commonly used in a reaction environment in which regeneration of targeting sites can occur shortly following a round of D-loop stimulated synthesis. This will avoid a stalling of amplification or inefficient linear amplification of ssDNA caused by oscillating single-sided synthesis from one end to the other.

In some embodiments, amplification of an analyte for subsequent detection the systems and devices of the present disclosure is achieved by loop-mediated isothermal amplification (LAMP). LAMP is described in U.S. Pat. No. 6,410,278, herein incorporated by reference. Generally, LAMP uses 4-6 primers recognizing 6-8 distinct regions of the target nucleic acid. A strand-displacing DNA polymerase initiates synthesis and two of the primers form loop structures to facilitate subsequent rounds of amplification. A person skilled in the art will be able to recognize and carry out variations on the general LAMP method as described above.

In some embodiments, amplification of an analyte for subsequent detection on the system and devices of the present disclosure is achieved by helicase-dependent amplification (HDA). HDA is based on the unwinding activity of a DNA helicase. HDA relies on one or more helicases to separate (melt, or unwind) two strands of a target nucleic acid duplex. HDA further utilizes a DNA or RNA polymerase to extend primers which are hybridized to single stranded nucleotide sequences to form complementary primer extension products. This process repeats itself so that exponential amplification can be achieved at a single temperature. A person skilled in the art will be able to recognize and carry out variations on the general HDA method as described above. “Complementary” as used herein refers to the complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

“Helicase” as used herein refers to any enzyme capable of enzymatically unwinding a double stranded nucleic acid. Any helicase that translocates along DNA or RNA in a 5′ to 3′ direction or in the opposite 3′ to 5′ direction may be used. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2nd ed. (1992)), include E. coli helicase I, II, III, & IV, Rep, DnaB, PriA, PcrA, T4 Gp41helicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem., 2001. 276:232-243), thermostable UvrD helicases from T. tengcongensis and T. thermophilus (Collins and McCarthy, Extremophiles. 2003, 7:35-41), thermostable DnaB helicase from T. aquaticus (Kaplan and Steitz, J. Biol. Chem., 1999. 274:6889-6897), and MCM helicase from archaeal and eukaryotic organisms (Grainge et al, Nucleic Acids Res., 2003. 31:4888-4898).

In certain embodiments, analyte, e.g., isolated total nucleic acid and/or enriched target nucleic acid, is segregated to a location in which target amplification occurs, e.g., the sample detection zones disclosed above or throughout the present disclosure. This may be done manually, or through automated methods, e.g., through the devices and methods disclosed herein.

In certain embodiments, the steps of target amplification of an analyte are carried out by the devices and methods of the present disclosure. In some cases, these steps are carried out by the devices of the present disclosures, at temperatures that allow for target amplification. Generally, these automated systems perform steps comprising: i) segregating isolated and/or enriched target nucleic acid, ii) adding enzymes and labelled primers or probes to a sample detection region, iii) adding activators if necessary for target amplification, iv) heating the reaction mixture and v) optionally, quenching the amplification reaction. The devices of the present disclosure that carry out the steps of target amplification in an isothermal target amplification reaction are substantially held at a single temperature, depending on the optimal temperature enzymes of various isothermal amplification reactions operate at. In some cases, target amplification carried out by the devices of the present disclosure can employ the RPA method, wherein the systems and devices are held at substantially a single temperature, e.g., at about 37° C., e.g., 35-37° C., 37-39° C., 36-38° C., 35-39° C., 32-42° C. For other isothermal methods, the systems and devices are held at a substantially single temperature, e.g., at a temperature of 40° C., or higher, e.g., 50° C.-70° C., such as 60° C.-65° C. In some cases, target amplification of a target nucleic acid sequence is carried out within 30 min, e.g., in about 25 min, 35 min, 27 min, 29 min, 31 min, 33 min, in at least 10 min e.g., 15 min or more, 20 min or more, 30 min or more.

In some cases, amplification of a target nucleic acid sequence is performed for a period of 30 min or less, e.g., 5 min, 10 min, 15 min, 20 min, or 25 min, e.g., 5 min-30 min, 5 min-25 min, 5 min-20 min, 5 min-25 min, 5 min-10 min, 10 min-30 min, 10 min-25 min, 10 min-20 min, 15 min-30 min, 15 min-25 min, or 15 min-20 min.

In some embodiments, amplification of a target nucleic acid is performed by isothermal amplification, e.g., LAMP, where the amplification is performed for 10 min or less, e.g., 9 min, 8 min, 7 min, 6 min, 5 min, 4 min, 3 min, 2 min, 1 min, 30 sec, 15 sec, or less, e.g., 15 sec-10 min, 30 sec-10 min, 1 min-10 min, 1 min-5 min, or 5 min-10 min.

In certain embodiments, the devices and methods disclosed herein detect a target nucleic acid present at a concentration as low as 1 aM. In certain embodiments, the the systems, devices, and methods disclosed herein detect a target nucleic acid present at a concentration of at least 1 aM or more, e.g., 10 aM, 100 aM, 1 fM, 10 fM, 100 fM, 1 pM, 10 pM, 100 pM, or more. In certain embodiments, the devices and methods disclosed herein detect an amplification product generated from an analyte, where the amplification product is present at a concentration of 25 aM-10 fM. Thus, the presently disclosed detection methods can detect an analyte present in a sample at a concentration of less than 1 aM, prior to amplification. In most instances, an amplification of only 15 times, 10 times, 5 times, 2 times or no amplification is needed to provide sufficient quantities of the target nucleic acid for detection.

In certain embodiments, the disclosed devices and methods can detect an amplification product produced from increasing copy number of the analyte by amplification, where as low as 1000 molecules of the amplification product are produced. Thus, in certain embodiments, a method for detecting presence of a target nucleic acid in a fluid sample may include amplifying the analyte in the sample by amplification to generate as low as 1000 molecules of an amplification product, wherein the amplifying incorporates a tag into the amplification product; capturing the amplification product on a plurality of capture objects comprising a binding member that specifically binds to the tag thereby generating a complex comprising capture object-amplification product; detectably labeling the amplification product in the complex to generate a detectably labeled complex; spatially segregating the capture objects into a plurality of wells such that each well contains no more than one capture object; and detecting the presence of the detectably labeled complex in the plurality of wells.

The primers or nucleotides used for the amplification reaction may be include a tag, such as, a hapten for incorporation of the tag into the amplified target nucleic acid. Any suitable tag may be utilized. For example, the tag may be a hapten for which a binding member that specifically binds to the hapten is available. For example, the hapten may be a small molecule for which antibodies that specifically bind to the hapten are available. Exemplary haptens include, avidin, biotin, digoxygenin, dinitrophenyl, dansyl-X, and derivatives thereof. In certain embodiments, the hapten may not be directly optically detectable, i.e., the hapten may not be a dye or a fluorescent molecule because such a hapten may interfere with the digital counting.

In accordance with the disclosed subject matter, and as embodied herein, the devices and methods disclosed herein for analyte can include isothermal amplification methods that rely on nicking and extension amplification reactions (NEAR) to amplify shorter sequences in a quicker timeframe than traditional amplification reactions. These methods can include, for example, reactions that use only two amplification oligonucleotides, one or two nicking enzymes, and a polymerase, under isothermal conditions.

Typically, in nicking and extension amplification, a target nucleic acid sequence, having a sense and antisense strand, is contacted with a pair of amplification oligonucleotides. The first amplification oligonucleotide comprises a nucleic acid sequence comprising a recognition region at 3′ end that is complementary to the 3′ end of the target sequence anti sense strand, a nicking enzyme site upstream of said recognition region, and a stabilizing region upstream of said nicking enzyme site (see, e.g., U.S. Pat. Nos. 9,689,031; 9,617,586; 9,562,264; and 9,562,263, each of which is incorporated herein by reference in its entirety). The second amplification oligonucleotide comprises a nucleotide sequence comprising a recognition region at 3′ end that is complementary to the 3′ end of the target sequence sense strand, a nicking enzyme site upstream of said recognition region, and a stabilizing region upstream of said nicking enzyme site. Two nicking enzymes are provided. One nicking enzyme is capable of nicking at the nicking enzyme site of the first amplification oligonucleotide but incapable of nicking within said target sequence. The other nicking enzyme is capable of nicking at the nicking enzyme site of the second amplification oligonucleotide but incapable of nicking within said target sequence. A DNA polymerase is employed under conditions for amplification which involves multiple cycles of extension of the amplification oligonucleotides thereby producing a double-stranded nicking enzyme site which are nicked by the nicking enzymes to produce the amplification product. For example, see U.S. Pat. Nos. 9,689,031; 9,617,586; 9,562,264; 9,562,263; and 10,851,406 and U.S. patent application Ser. Nos. 15/467,893 and 16/243,829, each of which is incorporated herein by reference in its entirety.

In some embodiments, reactions use only two templates to prime, one or two nicking enzymes, and a polymerase, under isothermal conditions. In exemplary embodiments, the polymerase and the nicking enzyme are thermophilic, and the reaction temperature is significantly above the melting temperature of the hybridized target region. The nicking enzyme nicks only one strand in a double-stranded duplex, so that incorporation of modified nucleotides is not necessary as it is in strand displacement. In some embodiments, the method is able to amplify RNA without a separate reverse transcription step, although conversion of RNA to DNA by reverse transcription may be used if desired.

In some embodiments, the method comprises contacting a target DNA molecule comprising a double-stranded target sequence having a sense strand and an antisense strand, with a forward template and a reverse template, wherein said forward template comprises a nucleic acid sequence comprising a recognition region at 3′ end that is complementary to the 3′ end of the target sequence anti sense strand; a nicking enzyme site upstream of said recognition region, and a stabilizing region upstream of said nicking enzyme site; the reverse template comprises a nucleotide sequence comprising a recognition region at 3′ end that is complementary to the 3′ end of the target sequence sense strand, a nicking enzyme site upstream of the recognition region, and a stabilizing region upstream of the nicking enzyme site; providing a first nicking enzyme that is capable of nicking at the nicking enzyme site of the forward template, and does not nick within the target sequence; providing a second nicking enzyme that is capable of nicking at the nicking enzyme site of the reverse template and does not nick within the target sequence; and providing a DNA polymerase; under conditions wherein amplification is performed by multiple cycles of the polymerase extending the forward and reverse templates along the target sequence producing a double-stranded nicking enzyme site, and the nicking enzymes nicking at the nicking enzyme sites, producing an amplification product.

In certain embodiments, the DNA polymerase is a thermophilic polymerase. In other examples, the polymerase and said nicking enzymes are stable at temperatures up to 37° C., 42° C., 60° C., 65° C., 70° C., 75° C., 80° C., or 85° C. In certain embodiments, the polymerase is stable up to 60° C. The polymerase may, for example, be selected from the group consisting of Bst (large fragment), 9° N, VentR® (exo-) DNA Polymerase, THERMINATOR, and THERMINATOR II (New England Biolabs).

The nicking enzyme may, for example, nick upstream of the nicking enzyme binding site, or the nicking enzyme may nick downstream of the nicking enzyme binding site. In certain embodiments, the forward and reverse templates comprise nicking enzyme sites recognized by the same nicking enzyme and the first and the second nicking enzyme are the same. The nicking enzyme may, for example, be selected from the group consisting of Nt.BspQI, Nb.BbvCi, Nb.BsmI, Nb.BsrDI, Nb.BtsI, Nt.AlwI, Nt.BbvCI, Nt.BstNBI, Nt.CviPII, Nb.BpulOI, and Nt.Bpul0I.

In some embodiments, the target sequence comprises from 1 to 5 nucleotides more than the sum of the nucleotides of said forward template recognition region and said reverse template recognition region.

In some embodiments, the forward template is provided at the same concentration as the reverse template. In other examples, the forward template is provided at a ratio to the reverse template at the range of ratios of 1:100 to 100:1.

As embodied herein, the NEAR reaction time can be about 10 minutes to about 30 minutes, or about 8 minutes to about 25 minutes, or about 8 minutes to about 20 minutes, or even about 8 minutes to about 15 minutes from the addition of the reagents sufficient to initiate NEAR amplification. In certain embodiments, the NEAR reaction time is about 1 minute to about 20 minutes, about 5 minutes to about 20 minutes, about 8 minutes to about 20 minutes, about 1 minute to about 10 minutes or about 5 minutes to about 10 minutes from the addition of the reagents sufficient to initiate NEAR amplification.

Provided herein are methods for measuring or detecting an analyte present in a biological sample. In some embodiments, the target analyte is a target nucleic acid. In some embodiments, the target nucleic acid is DNA. In some embodiments, the DNA is single stranded DNA. In some embodiments, the DNA is double stranded DNA. In some embodiments, the target nucleic acid is RNA. In such methods, a sample comprising a target analyte is deposited into a primary zone. In some embodiments, the primary zone contains microparticles or microparticles and assisting particles. Optionally, a lysis buffer may be added to the sample deposited in the primary. Following sample deposit, microparticles attached to a first specific binding partner that specifically binds to the target nucleic acid present in the sample are added to the sample. In some embodiments, the specific binding member is a nucleic acid that specifically hybridizes to the target nucleic acid. In some embodiments, the microparticles are microparticles and assisting particles. The sample may be incubated with the microparticles or microparticles and assisting particles for a time sufficient to allow binding of the first specific binding partner to bind an analyte present in the sample thereby producing microparticles bound to the target analyte. Optionally, the sample comprising the target analyte may be mixed with the microparticles attached to the first specific binding partner or microparticles attached to the first specific binding partner and assisting particles. Mixing may be achieved using any of the mixing methods described above.

Next, the microparticles bound to the target analyte or microparticles bound to the target analyte and assisting particles may be subjected to a magnetic field to move the microparticles bound to the target analyte or microparticles bound to the target analyte and assisting particles to one or more primary zones containing wash buffer. Next, the microparticles bound to the target analyte or microparticles bound to the target analyte and assisting particles may be subjected to a magnetic field to move the microparticles or microparticles and assisting particles to the detection zone in a sample analysis region. The microparticles bound to the target analyte may be allowed to settle into the array of wells in the detection zone. The microparticles may settle using gravitational force or by applying electric or magnetic force. Next, reagents necessary for the amplification and detection of the target analyte are added to the detection zone. The reagents necessary for the amplification and detection of the target analyte may be any of the reagents discussed above. Suitable reagents include, without limitation, a polymerase, a reverse transcriptase, a strand-displacing polymerase, forward and reverse primers specific to the target analyte, a recombinase, a single-stranded DNA binding protein, dNTPs, at least four primers that recognize at least 6 distinct regions in the target analyte, one or more helicases, a detection probe that produces a detectable signal following amplification of the target analyte, a first amplification oligonucleotide comprising a first nicking enzyme site, a second amplification oligonucleotide comprising a second nicking enzyme site, a first nicking enzyme that recognizes the first nicking enzyme site and second nicking enzyme recognizing the second enzyme site, etc. The reagents may also include reagents that elute the target analyte from the microparticles such that the target analyte is no longer bound to the first specific binding member. Following the addition of the reagents necessary for the amplification and detection of the target analyte to the detection zone, the wells containing the microparticles hybridized to the target analyte are sealed using a hydrophobic liquid (e.g., oil). The detection zone is then subject to conditions suitable for an isothermal amplification reaction such as those described above thereby producing a detectable signal in the wells. Lastly, the wells are imaged thereby detecting the detectable signal produced by the detection probe.

In some embodiments, the reagents necessary for the amplification and detection of the target analyte are for RPA. When the reagents are for RPA, the reagents comprise dNTPs, a recombinase, a single-stranded DNA binding protein, a strand-displacing polymerase, a forward and reverse primer specific to the target analyte, and a detection probe that produces a detectable signal following amplification of the target analyte. In some embodiments, the reagents necessary for the amplification and detection of the target analyte are for LAMP. When the reagents are for LAMP, the reagents comprise dNTPs, at least four primers that recognize at least 6 distinct regions in the target analyte, a strand-displacing polymerase, and a detection probe that produces a detectable signal following amplification of the target analyte. In some embodiments, the reagents necessary for the amplification and detection of the target analyte are for HDA. When the reagents are for HDA, the reagents comprise dNTPs, one or more helicases, a forward and reverse primer specific to the target analyte, a polymerase, and a detection probe that produces a detectable signal following amplification of the target analyte. In some embodiments, the reagents necessary for the amplification and detection of the target analyte are for NEAR. When the reagents are for NEAR, the reagents comprise dNTPs, a DNA polymerase, a first amplification oligonucleotide comprising a first nicking enzyme site, a second amplification oligonucleotide comprising a second nicking enzyme site, a first nicking enzyme that recognizes the first nicking enzyme site and second nicking enzyme recognizing the second enzyme site, and a detection probe that produces a detectable signal following amplification of the target analyte.

In some embodiments, an immunoassay and nucleic acid analysis are performed on the same sample on the same device. In these embodiments, a first set of microparticles or microparticles and assisting particles are added to the sample in the primary zone that are specific to the protein target analyte and bind to the protein target analyte. The first set of microparticles or microparticles and assisting particles are then moved to a different primary zone than the sample. A second set of microparticles or microparticles and assisting particles are added to the sample in the primary zone that are specific to the nucleic acid target analyte and bind to the nucleic acid target analyte. The first set of microparticles or microparticles and assisting particles are processed as discussed about (e.g., as discussed in the immunoassay section). The second set of microparticles or microparticles and assisting particles are then processed as discussed above (e.g., as discussed in the nucleic acid analysis section). Both the first set and second set of microparticles or microparticles and assisting particles are moved to the same detection zone or two separate detection zones and analyzed as discussed above.

1-114. (canceled)

115. A device, comprising:
a first substrate, and
a second substrate positioned on the first substrate, wherein the second substrate comprises a sidewall about at least a portion of a periphery of the second substrate, wherein the first substrate, sidewall, and second substrate define a central chamber therebetween,
wherein the second substrate comprises a surface facing the central chamber comprising a plurality of recessed elements, and a plurality of protruding elements;
wherein primary zones are defined between a surface of the plurality of protruding elements facing the first substrate and a surface of the first substrate facing the second substrate; and
wherein secondary zones are defined between a surface of the plurality of recessed elements facing the first substrate and the surface of the first substrate facing the second substrate, and
wherein the second substrate has an opening in one or more of the primary zones.

116. (canceled)

117. The device of claim 115, wherein primary zones are discrete.

118. The device of claim 115, wherein secondary zones are connected.

119. The device of claim 115, wherein the second substrate has an opening in one or more of the secondary zones.

120. The device of claim 115, wherein the second substrate has an opening in each of the plurality of primary zones.

121. The device of claim 115, wherein the first substrate has uniform hydrophobicity, the second substrate has uniform hydrophobicity, or the first substrate has uniform hydrophobicity and the second substrate has uniform hydrophobicity.

122-127. (canceled)

128. The device of claim 115, wherein the device comprises nine or more primary zones separated by twelve or more secondary zones.

129-130. (canceled)

131. The device of claim 115, wherein the device comprises eighteen or more primary zones separated by twenty-seven or more secondary zones.

132-133. (canceled)

134. The device of claim 115, further comprising a sample analysis region configured to analyze a sample.

135. (canceled)

136. The device of claim 134, wherein the sample analysis region is physically separated from the central chamber.

137. The device of claim 134, wherein the sample analysis region is not physically separated from the central chamber.

138. (canceled)

139. The device of claim 134, wherein the sample analysis region has a first end laterally separated from a second end and is defined by the surface of the second substrate facing the first substrate and the surface of the first substrate facing the second substrate.

140. The device of claim 139, wherein the surface of the second substrate facing the first substrate in the sample analysis region comprises an enlarged protruding element wherein:
a surface of the enlarged protruding element facing the first substrate has a greater surface area than a surface of the plurality of protruding elements facing the first substrate, and
the enlarged protruding element extends from the first end to the second end.

141. The device of claim 139, wherein the sample analysis region comprises a tertiary zone located at a midpoint between the first end and the second end of the of the sample analysis region wherein the tertiary zone is defined by:
a) the surface of the second substrate facing the first substrate and the surface of the first substrate facing the second substrate, or
b) the surface of the enlarged protruding element facing the first substrate and the surface of the first substrate facing the second substrate;
wherein the tertiary zone comprises at least one of selected from the group consisting of: one or more wells in the surface of the first substrate facing the second substrate, one or more nanopores, and one or more chambers.

142-146. (canceled)

147. The device of claim 115, wherein the plurality of primary zones are unbounded around the perimeter of each primary zone.

148. The device of claim 134, wherein the sample analysis region comprises a hydrophilic liquid well or reservoir wherein the hydrophilic liquid well or reservoir comprises a cylindrical opening spanning the second substrate and the hydrophilic liquid well or reservoir is located at the second end of the sample analysis region.

149. The device of claim 134, wherein the sample analysis region comprises a hydrophobic liquid well or reservoir wherein the hydrophobic liquid well or reservoir is an opening spanning the second substrate and the hydrophobic liquid well or reservoir is located at the first end of the sample analysis region.

150-157. (canceled)

158. The device of claim 115, wherein the device comprises one or more fixed primary zones wherein the one or more fixed primary zones are formed from a laterally extended protruding element in the second substrate facing the first substrate wherein the laterally extended protruding element has a surface facing the first substrate that has a greater surface area than the surface of the plurality of protruding elements facing the first substrate.

159-162. (canceled)

163. The device of claim 115, wherein the primary zones are in a pattern selected from the group consisting of a grid, a line, a non-grid, and a honeycomb pattern.

164-172. (canceled)

173. The device of claim 158, wherein the fixed primary zone comprises a hooked portion joined to a surface of the extended protruding element opposite the surface of the second substrate facing the first substrate.

174. The device of claim 158, wherein the fixed primary zone does not comprise a hooked portion.

175-302. (canceled)

303. A method of measuring or detecting a target analyte in a sample, the method comprising:
a) depositing the sample comprising the target analyte in an opening of a primary zone of a device;
b) depositing microparticles attached to a first specific binding partner that specifically binds to the target analyte in the opening of the primary zone comprising the sample thereby producing microparticles bound to the target analyte,
c) subjecting the microparticles bound to the target analyte to a magnetic field to move the microparticles to a primary zone containing a second specific binding partner that is detectably labeled thereby producing microparticles bound to detectably labeled target analyte;
d) subjecting the microparticles bound to the detectably labeled target analyte to a magnetic field to move the microparticles to one or more primary zones containing a wash buffer;
e) subjecting the microparticles bound to the detectably labeled target analyte to a magnetic field to move the microparticles to a detection zone containing wells in the sample analysis region;
f) adding a hydrophilic liquid to the wells containing the microparticles that react with the detectably labeled second specific binding partner to produce a detectable signal,
g) sealing the wells using a hydrophobic liquid; and
h) imaging the wells thereby detecting the detectable signal produced by the hydrophilic reacting with the second specific binding member;
wherein the device comprises:
a plurality of primary zones defined by a surface of a plurality of protruding elements on a surface of a second substrate facing a first substrate and a surface of the first substrate facing the second substrate, and
a plurality of secondary zones defined by a surface of a plurality of recessed elements on the surface of the second substrate facing the first substrate and a surface of the first substrate facing the second substrate,
wherein the second substrate has an opening in one or more of the primary zones and an opening in one or more of the secondary zones.

304. The method of claim 303, wherein the target analyte is a protein.

305. The method of claim 303, wherein the first and second specific binding members are antibodies that specifically bind to different epitopes on the target analyte.

306-308. (canceled)

309. The method of claim 303, further comprising:
i) depositing lysis buffer in the in the opening of the primary zone comprising the sample, wherein step i) occurs between steps a) and b)

310. The method of claim 303, further comprising:
j) mixing the primary zone comprising the sample and the microparticles, wherein step j) occurs between steps b) and c).

311. The method of claim 303, further comprising:
k) subjecting the microparticles bound to the target analyte to a magnetic field to move the microparticles to one or more primary zones containing a wash buffer, wherein step k) occurs between steps b) and c).

312-313. (canceled)

314. The method of claim 303, wherein step B) further comprises depositing assisting particles.

315. The method of claim 303, wherein the device is the device of claim 115.

316-381. (canceled)
